SEQUENCE LISTING (33 sequences)

SEQ ID NO: 1 - protein, 15 aa, synthetic construct
KDIVALMVRRAYDIA

SEQ ID NO: 2 - protein, 16 aa, synthetic construct
ILNWVKFKAQVQLNKK

SEQ ID NO: 3 - protein, 17 aa, synthetic construct
KKWKVKYYKGLGTSTSK

SEQ ID NO: 4 - protein, 15 aa, synthetic construct
ISIWNNGKGIPVVEH

SEQ ID NO: 5 - protein, 17 aa, synthetic construct
SILNWVKFKAQVQLNKK

SEQ ID NO: 6 - protein, 17 aa, synthetic construct
SILNWVKFKAQVQLNKK

SEQ ID NO: 7 - protein, 17 aa, synthetic construct
AERVGLHKVFKLQTSLT

SEQ ID NO: 8 - protein, 15 aa, synthetic construct
DPENNLISIWNNGKG

SEQ ID NO: 9 - protein, 15 aa, synthetic construct
NNLISIWNNGKGIPV

SEQ ID NO: 10 - protein, 15 aa, synthetic construct
KQTWMDNMGRAGEME

SEQ ID NO: 11 - protein, 15 aa, synthetic construct
DKDIVALMVRRAYDI

SEQ ID NO: 12 - protein, 15 aa, synthetic construct
IVALMVRRAYDIAGS

SEQ ID NO: 13 - protein, 15 aa, synthetic construct
VESILNWVKFKAQVQ

SEQ ID NO: 14 - protein, 15 aa, synthetic construct
VKYYKGLGTSTSKEA

SEQ ID NO: 15 - protein, 15 aa, synthetic construct
YKGLGTSTSKEAKEY

SEQ ID NO: 16 - protein, 17 aa, synthetic construct
FTMLSSLARLLFPPKDD

SEQ ID NO: 17 - protein, 15 aa, synthetic construct
TLKFLYDDNQRVEPE

SEQ ID NO: 18 - protein, 15 aa, synthetic construct
PMVLINGAEGIGTGW

SEQ ID NO: 19 - protein, 15 aa, synthetic construct
VAILNSTTIEISELP

SEQ ID NO: 20 - protein, 9 aa, synthetic construct
GINYREVTF

SEQ ID NO: 21 - protein, 9 aa, synthetic construct
TVETASREY

SEQ ID NO: 22 - protein, 9 aa, synthetic construct
SLKVIHEQV

SEQ ID NO: 23 - protein, 9 aa, synthetic construct
VTKLVDVVK

SEQ ID NO: 24 - protein, 9 aa, synthetic construct
SILNWVKFK

SEQ ID NO: 25 - protein, 9 aa, synthetic construct
YLYGQTTTY

SEQ ID NO: 26 - protein, 9 aa, synthetic construct
MTIINLAQN

SEQ ID NO: 27 - protein, 9 aa, synthetic construct
VLFDHVGCL

SEQ ID NO: 28 - protein, 15 aa, synthetic construct
AAALCHFTSIEVVKK

SEQ ID NO: 29 - protein, 15 aa, synthetic construct
VVKSTSPVKLKTFKK

SEQ ID NO: 30 - protein, 15 aa, synthetic construct
SHNLRGVIPRSFEYL

SEQ ID NO: 31 - protein, 197 aa, synthetic construct
MRVTAPRTLILLLSGALALTETWAGGSGGGGSGGGIVESILNWVKFKAQVQLNKKCSAVKGGSGGGGSGGMQSLDKDIVALMVRRAYDIAGSTKDGGSGGGGSGGSTPNHKKWKVKYYKGLGTSTSKEAKEYGGSGGGGSGGIVGIVAGLAVLAVVVIGAVVATVMCRRKSSGGKGGSYSQAASSDSAQGSDVSLTA

SEQ ID NO: 32 - protein, 9 aa, synthetic construct
VALMVRRAY

SEQ ID NO: 33 - protein, 9 aa, synthetic construct
VKFKAQVQL

(19) United States
(12) Patent Application Publication
(10) Pub. No.: US 2026/0284165 A1
(43) Pub. Date: Sep. 24, 2026

(54) COMPOSITIONS AND METHODS FOR TREATMENT OR PRVENTATIVE TREATMENT OF CANCER INCLUDING TRIPLE NEGATIVE BREAST CANCER AND/OR LUNG CANCER

(71) Applicant: The Medical College of Wisconsin, Inc., Milwaukee, WI (US)

(72) Inventors: Ming You, Milwaukee, WI (US); Yian Wang, Milwaukee, WI (US); Jing Pan, Milwaukee, WI (US); Sang Beom Lee, Milwaukee, WI (US)

(21) Appl. No.: 19/473,075

(22) PCT Filed: Apr. 5, 2024
(86) PCT No.: PCT/US2024/023404

§ 371 Date: Oct. 6, 2025

Related U.S. Application Data
(60) Provisional application No. 63/495,005, filed on Apr. 7, 2023.

(51) Int. Cl. A61K 39/00; A61P 35/00; A61P 37/04

(52) U.S. Cl. CPC A61K 39/001154; A61P 35/00; A61P 37/04; A61K 2039/53; A61K 2039/55555; A61K 2039/55561; A61K 2039/572; A61K 2039/70; A61K 2039/812; A61K 2039/86; C12Y 599/01003

(57) ABSTRACT

Disclosed herein are compositions and methods for treating, and for the preventative treatment of, a subject diagnosed with or at risk of breast cancer, including triple negative breast cancer, and/or a subject diagnosed with or at risk of lung cancer.

### CROSS-REFERENCE TO RELATED APPLICATIONS

This application claims priority to U.S. Application Ser. No. 63/495,005, filed on Apr. 7, 2023, the entire contents of which are hereby incorporated by reference.

### STATEMENT REGARDING FEDERALLY SPONSORED RESEARCH OR DEVELOPMENT

None.

### REFERENCE TO AN ELECTRONIC SEQUENCE LISTING

The contents of the electronic sequence listing (650053.01062.xml; Size: 29,855 bytes; and Date of Creation: Apr. 5, 2024) is herein incorporated by reference in its entirety.

### BACKGROUND

Breast cancer is the leading malignancy in women with 281,550 estimated new cases and 43,600 estimated deaths reported in 2021 in the United States. Approximately 20% of breast cancers are classified as triple negative breast cancer (TNBC) as they do not express estrogen receptor (ER), progesterone receptor (PR) and human epidermal growth factor receptor 2 (HER2), encompassing the molecular subtypes of basal-like and the more recently defined claudin-low group. TNBC is also associated with African American race, younger age, higher grade and mitotic index, and more advanced stage at diagnosis.

### SUMMARY

Disclosed herein are compositions and methods for the treatment of a subject and/or preventative treatment of a subject.

In various aspects, the methods for the treatment of a subject and/or preventative treatment of a subject are for the treatment and/or preventative treatment of a cancer, including breast cancer and/or lung cancer.

In various aspects, the methods for the treatment of a subject and/or preventative treatment of a subject are for the treatment and/or preventative treatment of a breast cancer, including TNBC.

In various aspects, the methods for the treatment of a subject and/or preventative treatment of a subject are for the treatment and/or preventative treatment of lung cancer.

Disclosed herein are one or more polypeptides comprising or consisting of fragments of topoisomerase 2 alpha (Top2A). In various aspects, the one or more polypeptides comprise or consist of one or more of: a polypeptide that is at least 80%, 85%, 90%, 95%, or 99% identical to SEQ ID NO: 1; a polypeptide that is at least 80%, 85%, 90%, 95%, or 99% identical to SEQ ID NO: 2; or a polypeptide that is at least 80%, 85%, 90%, 95%, or 99% identical to SEQ ID NO: 3. In one or more aspects, the one or more polypeptides comprise or consist of a polypeptide having the sequence of SEQ ID NO: 1, a polypeptide having the sequence of SEQ ID NO: 2, and a polypeptide having the sequence of SEQ ID NO: 3.

Disclosed herein are compositions comprising one or more polypeptides. In various aspects, the one or more polypeptides comprise or consist of one or more of: a polypeptide that is at least 80%, 85%, 90%, 95%, or 99% identical to SEQ ID NO: 1; a polypeptide that is at least 80%, 85%, 90%, 95%, or 99% identical to SEQ ID NO: 2; or a polypeptide that is at least 80%, 85%, 90%, 95%, or 99% identical to SEQ ID NO: 3. In one or more aspects, the one or more polypeptides comprise or consists of a polypeptide having the sequence of SEQ ID NO: 1, a polypeptide having the sequence of SEQ ID NO: 2, a polypeptide having the sequence of SEQ ID NO: 3. In one or more aspects, the composition can include, or optionally include, an adjuvant.

Also disclosed herein are compositions comprising one or more polynucleotides. In various aspects, the one or more polynucleotides encode for one or more of: a polypeptide that is at least 80%, 85%, 90%, 95%, or 99% identical to SEQ ID NO: 1; a polypeptide that is at least 80%, 85%, 90%, 95%, or 99% identical to SEQ ID NO: 2; or a polypeptide that is at least 80%, 85%, 90%, 95%, or 99% identical to SEQ ID NO: 3.

Also disclosed herein are methods for treating a subject, or methods for the preventative treatment of the subject, comprising administering to the subject a composition comprising one or more polypeptides. In various aspects, the one or more polypeptides comprise or consist of one or more of: a polypeptide that is at least 90%, 95%, or 99% identical to SEQ ID NO: 1; a polypeptide that is at least 90%, 95%, or 99% identical to SEQ ID NO: 2; or a polypeptide that is at least 90%, 95%, or 99% identical to SEQ ID NO: 3. In one or more aspects, the one or more polypeptides comprise or consist of a polypeptide having the sequence of SEQ ID NO: 1, a polypeptide having the sequence of SEQ ID NO: 2, and/or a polypeptide having the sequence of SEQ ID NO: 3. In one or more aspects, the composition can include, or optionally include, an adjuvant. In some aspects, the subject has not previously been diagnosed with, or treated for, cancer, breast cancer, or TNBC. In various aspects, the subject has been previously diagnosed with cancer, breast cancer, and/or TNBC. In one or more aspects, a subject has been diagnosed with breast cancer, where the breast cancer does not express one or more of: an estrogen receptor (ER), a progesterone receptor (PR), or human epidermal growth factor 2 (HER2). In various aspects, the subject exhibits one or more risk factors associated with TNBC selected from pregnancy, multiple child births, and obesity. In certain aspects, at least one, or at least two doses of a therapeutically effective amount of the composition is administered to the subject.

### BRIEF DESCRIPTION OF THE FIGURES

FIGS. 1A-1C. Depict data associated with the overexpression of the Top2A gene in mouse and human TNBC tissues. (FIG. 1A) Overexpression of the Top2A gene in M6 mouse mammary carcinoma cells vs. M28 normal mammary cells; both cell lines were derived from C3 (1)/Tag mice. (FIG. 1B) Overexpression of the Top2A gene in human TNBCs. (FIG. 1C) Overexpression of the Top2A gene in African American (AA) TNBCs.

FIGS. 2A-2B. Depict data associated with overexpression of Top2A protein confirmed in human breast cancer tissue microarrays and mouse TNBCs derived from C3 (1)/Tag mice. (FIG. 2A) IHC staining of Top2A in breast cancer specimens in two types of human breast TMAs (BRC961 and 962). a. Comparison of IHC staining results between normal and malignant tissues. **P<0.01. b. Comparison of IHC staining results on all tissue, Normal, Inflammatory, Hyperplasia, Benign, In-situ, Malignant and Sarcoma. c. Representative IHC staining score index of Top2A in a human breast TMA. (FIG. 2B) Representative images of IHC staining of Top2A on 20 weeks old C3 (1)/Tag mammary gland tissues. a. Mammary gland tissue from a wild-type C3 (1)/Tag mouse littermate. b-c. Mammary gland tissues from C3 (1)/Tag mouse #1 and mouse #3, respectively. All images were scanned and captured by NanoZoomer system (Hamamatsu Photonics, Hamamatsu, Japan). (FIG. 2C) Western blot analysis of protein expression in murine breast cancer cell lines. (a) Representative images of Western blots. (b) Protein expression levels of Top2A were assessed via Western blot analysis on the following cell lines: M28, the normal control; M27, weakly tumorigenic/benign tumor; M6, malignant tumor; M6C, metastatic tumor.

FIGS. 3A and 3B. Depict data associated with an in vivo screen for Top2A peptides by ELISPOT in C3 (1)/Tag-REAR mice. (FIG. 3A) Representative immunogenic heatmap for Top2A in FVB mice. The complete mouse Top2A protein sequence shows identified immunogenic “hot-spots” for FVB mice. Colors represent the percent of the highest score from three algorithms for each amino acid from dark red to light blue in the order of rank scores. Color strata are as follows: dark red≥75% of the highest score; red=50-75% of the highest score; orange=40-50% of highest score; yellow=30-40% of the highest score; green=20-30% of the highest score; blue≤20% of the highest score. (FIG. 3B) Selected peptide sequences of the human Top2A based on a combined scoring system. In descending order, SEQ ID NOs: 1, 2, 3. (FIG. 3C) IFN-γ based ELISpot assay results. Splenocytes were collected from vaccinated mice and pulsed with negative control peptide (HIV peptide), positive control peptide (Concanavalin A), 3 individual Top2A peptides, or their combination (combo). After 72 h of incubation, the ELISpot assay was performed, plates were scanned, and spot numbers were statistically analyzed. Data are shown as the mean±SE of three replicate wells per group, n=5, ***, p<0.001.

FIGS. 4A-4D. Depict data associated with the in vivo immunogenicity of Top2A peptides. (FIGS. 4A & 4B) Demonstration of Top2A peptide specificity using peptide-loaded MHC class II tetramers. A. Top2A peptide-specific tetramer staining of splenic CD4 T cells from Top2A vaccinated mice. *p<0.05. B. Representative flow cytometric plots of Top2A tetramer+CD4 T cells. (FIGS. 4C & 4D) Top2A peptides induced the proliferation of splenic CD4+ T cells isolated from vaccinated mice. C. Isolated CD4+ T cells were CFSE-labeled and stimulated with mature DCs pulsed with Top2A peptides. *p<0.05, **p<0.01, ***p<0.001. D. Representative flow cytometric histogram of CFSE stained CD4+ T cells. Percentages are indicated as frequency of dividing CD4+ T cells.

FIGS. 5A-5D. Depict data demonstrating Top2A vaccination inhibited tumor growth in C3 (1)/Tag-REAR mice. (FIG. 5A) Experimental design and timeline of vaccine administration. (FIG. 5B) Tumor growth curves after M6 tumor cell inoculation. Following implantation, tumor diameters were measured using a digital caliper and tumor volumes were calculated using the formula: maximum diameter×(minimum diameter)²×0.4. Data are shown as the mean±SE, *P<0.05, **P<0.01, ***P<0.001. (FIG. 5C) The weight of each tumor was taken at the experimental endpoint. Data are shown as the mean±SE, n=6-7 mice per group, *P<0.05. (FIG. 5D) Top2 A vaccination significantly increased the percentages of functionally activated CD4+ and CD8+ cells in the spleen. Cells isolated from the spleens of Top2A vaccinated and adjuvant control mice were stained intracellularly for granzyme B, IFN-γ and TNFα. *P<0.05, **P<0.01.

FIGS. 6A-6D. Depict data demonstrating that Top2A vaccination of C3/Tag genetically engineered mice prevented breast tumor development. (FIG. 6A) Experimental design of the experiments. (FIG. 6B) Summary of tumor development in vaccinated C3/Tag genetically engineered mice at 20-weeks of age. Estimated tumor volumes are depicted based on development of palpable tumors; diameters were measured using a digital caliper and tumor volumes were calculated using the formula: maximum diameter×(minimum diameter)²×0.4. *P<0.05. (FIG. 6C) Immunohistochemistry (IHC) analyses were performed to determine infiltrating CD4 and CD8 T cells infiltrating tumor tissues. The number of positive cells in the field was expressed as #of CD4+ cells and CD8+ cells per mm2 tumor area. **p<0.01. (FIG. 6D) Representative images of H&E staining and IHC staining of CD4 and CD8 in the Top2A vaccinated tumors. All images were scanned using a NanoZoomer system (Hamamatsu Photonics, Hamamatsu, Japan).

FIG. 7. Depict data associated with cytokine production by splenic T cells from Top2A vaccinated mice. Twelve Cytokines were analyzed by an ELISArray Kit, QIAGEN. Splenocytes from either Top2A vaccinated or adjuvant alone mice were collected and cocultured with the Top2A vaccine for 72 hours. The supernatant was collected and assayed with QIAGEN's ELISArray Kit following the manufacturer's instructions. Data are shown as the mean±SE, CpG only (n=3), Top2A (n=3). *P<0.05, ***P<0.001 (compared with CpG only).

FIGS. 8A-8D. Depict data demonstrating that Top2A vaccinated C3 (1)/Tag mice were protected from M6 cell secondary challenge. (FIG. 8A) Schematic of the experimental design and timeline of vaccine administration. (FIG. 8B) Tumor growth curves after M6 tumor cell inoculation in C3 (1)/Tag mice at 22 weeks of age. 5×10⁵ cells were injected into the #4 mammary fat pads of female C3 (1)/Tag mice. Following implantation, tumor diameters were measured using a digital caliper, and tumor volumes were calculated using the formula: maximum diameter×(minimum diameter)²×0.4. Control C3 (1)/Tag female mice (n=3) were never vaccinated. Top2A C3 (1)/Tag female mice (n=3) were vaccinated with Top2A. At the time of M6 inoculation, there was no tumor present in the #4 mammary fat pads. (FIG. 8C) Weight of each tumor was taken at the end of the experiment. (FIG. 8D) Top2A vaccination increased the number of CD4+ and CD8+ tumor-infiltrating lymphocytes in treated animals. (a) Representative images of IHC staining of CD4 and CD8 on tumor tissue. (b) Quantification of CD8 IHC images. The number of positive cells in the field was expressed as #of CD4+ and CD8+ cells per mm2 tumor area. All images were scanned by NanoZoomer system (Hamamatsu Photonics, Hamamatsu, Japan). All data are shown as the mean±SE, *P<0.05, **P<0.01, ***p<0.001.

FIGS. 9A-9D. Depict data associated with clustering analysis of scRNA-seq data from TNBC and lymph node tissue immune cells sorted by flow cytometry. (FIG. 9A) The expression of the marker genes for the CD8+T, CD4+T, DNT, DC, macrophages and neutrophils in mouse TNBC tumor samples from control Top2A vaccinated mice. (FIG. 9B) Landscape of the overall immune cells populations from the tumor samples of control and Top2A vaccinated mice. (FIG. 9C) Expression of the marker genes for the CD8+T, CD4+T, DNT, DC and macrophages in the lymph node samples from control and Top2A vaccinated mice. (D) Landscape of the immune cells in lymph nodes of control and Top2A vaccinated mice.

FIGS. 10A-10H. Depicts data demonstrating the effects of the Top2A vaccine on CD8+ T cells subsets in breast tumor (FIGS. 10A-10D) and lymph node (FIGS. 10E-10H) tissues. (FIGS. 10A &10E) Canonical marker expression singled out CD8+ T cell subsets based on scRNA-seq data. (FIGS. 10B & 10F) Heatmap of the expression of the markers for each CD8+ T cell subset. (FIGS. 10C & 10G) The distribution of each CD8+ T cell subset; (FIGS. 10D & 10H) Percent changes in the CD8+ T cells subsets across the control and the TOP2A vaccine treatment groups.

FIGS. 11A-11H. Depict data demonstrating that Top2A vaccine treatment influenced changes in CD4+ T cell subsets in breast tumor (FIGS. 11A-11D) and lymph node (FIGS. 11E-11H) tissues. (FIGS. 11A & 11E) Canonical marker expression singled out CD4+ T cells based on scRNA-seq data. (FIGS. 11B & 11F) Heatmap of the expression of the markers for each CD4+ T cell subset. (FIGS. 11C & 11G) Distribution of each CD4+ T cell subset; (FIGS. 11D & 11H) Percent changes of the CD4+ T cells subsets across the control and the TOP2A vaccine treatment groups.

FIGS. 12A and 12B. Depict data associated with CD4+ T-cell receptor (TCR) clonotype distribution in the mouse breast tumor samples from Top2A vaccine treated and non-treated control mice. (FIG. 12A) Distribution of CD4+ TCR clonotypes in breast tumor tissues from Top2A vaccine treated mice. (FIG. 12B) Distribution of CD4+ TCR clonotypes in the breast tumor tissues from non-treated CpG control mice.

FIGS. 13A-13C. Depict data associated with TCR-peptide binding prediction for the three Top2A peptide epitopes assayed using tetramers. Predicted binding of the following peptides with the CD4+ TCR clonotypes detected in breast tumor tissues from the Top2A vaccinated mice: (A) First Top2A peptide-KDIVALMVRRAYDIA (SEQ ID NO: 1); (B) Second TOP2A peptide-ILNWVKFKAQVQLNKK (SEQ ID NO: 2); (C) Third TOP2A peptide-KKWKVKYYKGLGTSTSK (SEQ ID NO: 3).

FIGS. 14A-14D. Depicts data demonstrating the effects of Top2A vaccine treatment on the DNT cells (CD4-CD8-double negative T cells) in breast tumor samples. (FIG. 14A) Canonical T cell marker expression was used to generate UMAP plots from scRNA-seq data. (FIG. 14B) Heatmap of the expression of the markers for each DNT cell subset. (FIG. 14C) Distribution of each DNT cell subset. (FIG. 14D) Percent changes of the DNT cell subsets across the control and the Top2A vaccine treatment groups.

FIGS. 15A-15D. Depict data demonstrating the effects of Top2A vaccine treatment on DNT cell subsets in lymph node tissues. (FIG. 15A) Canonical marker expression on DNT cells was used to generate UMAP plots from scRNA-seq data. (FIG. 15B) Heatmap of markers expressed by each DNT cell subset. (FIG. 15C) UMAP plot distribution of each DNT cell subset. (FIG. 15D) Percent changes in the DNT cell subsets across the control and the TOP2A vaccine treatment groups.

FIGS. 16A-16D. Depict data demonstrating the effects of Top2A vaccine on macrophages in the breast tumor samples. (FIG. 16A) Canonical markers expressed on macrophages were used to generate UMAP plots based on scRNA-seq data. (FIG. 16B) Heatmap based on expression of the markers for each macrophage subset, M1 (anti-tumor) and M2 (pro-tumor) macrophages. (FIG. 16C) UMAP plot distribution of the M1 and M2 macrophage subsets. (FIG. 16D) Percent changes in the M1 and M2 macrophage subsets in control versus TOP2A vaccine treated mice.

FIGS. 17A-17D. Depict data demonstrating the effects of Top2A vaccine treatment on macrophages in lymph nodes. (FIG. 17A) Canonical markers expressed on macrophages were used to generate UMAP plots based on scRNA-seq data. (FIG. 17B) Heatmap based on expression of the markers for M1 and M2 macrophage subsets. (FIG. 17C) UMAP plot distribution of the M1 and M2 macrophage subsets. (FIG. 17D) Percent changes in the M1 and M2 macrophage subsets in control versus TOP2A vaccine treated mice.

FIGS. 18A-18D. Depict data demonstrating the effects of Top2A vaccine on dendritic cells (DC) in the breast tumor samples. (FIG. 18A) Canonical markers expressed on DC were used to generate UMAP plots based on scRNA-seq data. (FIG. 18B) Heatmap based on expression of the markers for each DC subset, conventional dendritic cells (CDC) and plasmacytoid dendritic cells (pDC). (FIG. 18C) UMAP plot distribution of the cDC and pDC subsets. (FIG. 18D) Percent changes in the cDC and pDC subsets in control versus TOP2A vaccine treated mice.

FIGS. 19A-19D. Depict data demonstrating the effects of Top2A vaccine treatment on DC in the lymph node samples. (FIG. 19A) Canonical markers expressed on DC were used to generate UMAP plots based on scRNA-seq data. (FIG. 19B) Heatmap based on expression of the markers for cDC and pDC subsets. (FIG. 19C) UMAP plot distribution of the cDC and pDC subsets. (FIG. 19D) Percent changes in the cDC and pDC subsets in control versus TOP2A vaccine treated mice.

FIGS. 20A-20D. Depict data demonstrating the effects of Top2A vaccine treatment on neutrophils in breast tumor samples. (FIG. 20A) Canonical markers expressed on neutrophils were used to generate UMAP plots based on scRNA-seq data. (FIG. 20B) Heatmap based on expression of the markers for the neutrophil subsets, Stage I and Stage II. (FIG. 20C) UMAP plot distribution of the Stage I and Stage II neutrophil subsets. (FIG. 20D) Percent changes in the Stage I and Stage II subsets in control versus TOP2A vaccine treated mice.

FIGS. 21A-21B. FIG. 21A depicts data associated with IFN-γ ELISPOT assays of the peptides as detailed in Example 13, showing the assay plate. FIG. 21B is a table listing the tested peptides.

FIG. 22. Depicts a bar graph of the IFN-γ ELISPOT assays of FIG. 21A.

FIGS. 23A-23F. Depict experimental design and data showing that the Top2A vaccine inhibited lung tumor progression in synergistic models of lung cancer. (FIGS. 23A and 23D) Experimental design and timeline of vaccine administration. (FIGS. 23B and 23E) Tumor growth curves after LKR13 (FIG. 23B) and LLC (FIG. 23E) tumor cell inoculation. Following implantation, tumor diameters were measured using a digital caliper and tumor volumes were calculated using the formula: maximum diameter×(minimum diameter)²×0.4. Data are shown as the mean±SE, **P<0.01, and p-values are calculated using one-way ANOVA. (FIGS. 23C and 23F) Survival analysis of LKR13 (FIG. 23C) and LLC (FIG. 23F) tumor cell inoculation mice (n=5). Statistical significance was calculated using log-rank (Mantel-Cox) test, ****P<0.0001.

FIGS. 24A-C. OVA & TOP2A mRNA vaccines in lung metastasis models. (FIG. 24A) The design of the mRNA vaccines. (FIG. 24B) C57BL/6 mice were inoculated with B16 melanoma cells and immunized with OVA-mRNA LNP with lung metastases as the endpoint. (FIG. 24C) SV129 mice were inoculated with LKR13 lung tumor cells and immunized with TOP2A-mRNA-LNP with lung metastases as the endpoint. ***P<0.001

FIG. 25. IEDB prediction of binding affinity for TOP2A. Binding affinity predicted by IEDB algorithm was plotted based on the percentile ranking of each amino acid, mapped to the amino acid sequence of TOP2A with red color indicates more alleles binding to the region.

FIG. 26. World population coverage calculated by IEDB.

FIGS. 27A-E. Expression of cyclin E and KIF15 in TNBCs from humans and C3 (1)/Tag mice, and immune response of selected epitopes. (FIG. 27A) Overexpression of cyclin E and KIF15 in human TNBCs; (FIGS. 27B & 27C) Overexpression of cyclin E in TNBCs from C3 (1)/Tag mice; (FIGS. 27D and 27E) Overexpression of KIF15 in TNBCs from C3 (1)/Tag mice

FIGS. 28A-C. Cyclin E2 and KIF15 vaccinations are effective in inhibiting growth of TNBC in the C3 (1) Tag-RARE syngeneic mouse model. (FIG. 28A), IFNγ ELISPOT in splenocytes after completion of four immunizations. HIV p52 was used as a negative control. N=5 mice/group; B&C, Mean tumor volume (mm3±SEM) from mice injected with adjuvant (CpG), cyclin E2 peptides (FIG. 28B) or KIF15 peptide (FIG. 28C) in C3 (1) Tag mice, n=5 mice/group; *P<0.05.

FIG. 29. The preventive effect on TNBC development of the combination with Cyclin E2, KIF15 and TOP2A vaccine in the C3 (1)/Tag GEM model. N=9-10 mice/group; * P<0.05.

FIGS. 30A-30K. TOP2A vaccination induced the immune response and prevented breast tumor development C3/Tag mice. (FIG. 30A) Representative immunogenic heatmap for TOP2A in FVB mice. The complete mouse TOP2A protein sequence shows identified immunogenic “hot-spots” for FVB mice. Colors represent the percent of the highest score from three algorithms for each amino acid from dark red to light blue in the order of rank scores. (FIG. 30B) Representative IFN-γ based ELISpot assay results showing T cell responses to specific TOP2A peptides from mouse splenocyte. (FIG. 30C) IFN-γ based ELISpot assay results. Splenocytes were collected from vaccinated mice and pulsed with negative control peptide (HIV peptide), positive control peptide (Concanavalin A), 3 individual TOP2A peptides, or their combination (combo). Data are shown as the mean±SE of three replicate wells per group, n=5, ****p<0.0001. (FIG. 30D) Experimental design of the experiments. (FIG. 30E) Summary of tumor development in vaccinated C3/Tag genetically engineered mice at 20-weeks of age. Estimated tumor volumes are depicted based on the development of palpable tumors. *p<0.05. (FIG. 30F) Representative IHC staining and quantitative data for tumor-infiltration CD4+ cell from mammary tumor tissues. (FIG. 30G) Representative IHC staining and quantitative data for tumor-infiltrating CD8+ cell from mammary tumor tissues. The number of positive cells in the field was expressed as #of CD4+ cells (FIG. 30F) and CD8+ cells (FIG. 30G) per mm2 tumor area. **p<0.01. (FIG. 30H) To evaluate bulk cytokine production by the T cells in culture supernatants. 12 Cytokines were analyzed by an ELISArray Kit, QIAGEN. Splenocytes from either TOP2A vaccinated or adjuvant alone mice were collected and cocultured with TOP2A vaccine for 72 hours, Adj (CpG only, n=3), TOP2A (n=3). (FIG. 30I-30K) Body weights, ALT and AST level at 20-weeks of age. Data are shown as the mean±SE, *p<0.05, ***p<0.001 (compare with CpG only, 2-tail t-test).

FIGS. 31A-31E. FIG. 2 TOP2A vaccination inhibited tumor growth in the syngeneic C3 (1)/Tag-REAR mouse model. (FIG. 31A) Experimental design and timeline of vaccine administration. (FIG. 31B) Tumor growth curves after M6 tumor cell inoculation. Following implantation, tumor diameters were measured using a digital caliper and tumor volumes were calculated using the formula: maximum diameter×(minimum diameter)²×0.4. Data are shown as the mean±SE, *p<0.05, **p<0.01, ***p<0.001 (2-tail t-test). (FIG. 31C) The weight of each tumor was taken at the experimental endpoint. Data are shown as the mean±SE, n=6-7 mice per group, *P<0.05. (FIG. 31D-31I) TOP2 A vaccination significantly increased the percentage of the functionally activated D4+ and CD8+ cells in the spleens. Cells isolated from the spleen were stained for the intracellular markers (granzyme B, IFN-γ and TNFα). *p

FIGS. 32A-32E. FIG. 3 TOP2A vaccinated C3 (1)/Tag mice were protected from M6 cell secondary challenge. (FIG. 32A) Schematic of the experimental design and timeline of vaccine administration. (FIG. 32B) Tumor growth curves after M6 tumor cell inoculation in C3 (1)/Tag mice at 22 weeks of age. 5×10⁵ cells were injected into the #4 mammary fat pads of female C3 (1)/Tag mice. Following implantation, tumor diameters were measured using a digital caliper, and tumor volumes were calculated using the formula: maximum diameter×(minimum diameter)²×0.4. Control C3 (1)/Tag female mice (n=4) were never vaccinated. C3 (1)/Tag female mice (n=3) were vaccinated with TOP2A. At the time of M6 inoculation, there was no tumor present in the #4 mammary fat pads. (FIG. 32C) Weight of each tumor was taken at the end of the experiment. (FIG. 32D-32E) TOP2A vaccination increased the number of CD4+ (FIG. 32D) and CD8+ (FIG. 32E) tumor-infiltrating lymphocytes in treated animals. The number of positive cells in the field was expressed as #of CD4+ and CD8+ cells per mm2 tumor area. All data are shown as the mean±SE, *p<0.05, **p<0.01, ***p<0.001 (2-tail t-test).

FIGS. 33A-33P. FIG. 4 Effects of the TOP2A vaccine on CD8+T and CD4+ T cell subsets in breast tumor and lymph node tissues. (FIG. 33A, 33E) Canonical marker expression singled out CD8+ T cell subsets based on scRNA-seq data. (FIG. 33B, 33F) Heatmap of the expression of the markers for each CD8+ T cell subset. (FIG. 33C, 33G) The distribution of each CD8+ T cell subset; (FIG. 33D, 33H) Percent changes in the CD8+ T cell subsets across the control and the TOP2A vaccine treatment groups. (FIG. 33I, 33M) Canonical marker expression singled out CD4+ T cells based on scRNA-seq data. (FIG. 33J, 33N) Heatmap of the expression of the markers for each CD4+ T cell subset. (FIG. 33K, 33O) Distribution of each CD4+ T cell subset; (FIG. 33L, 33P) Percent changes of the CD4+ T cell subsets across the control and the TOP2A vaccine treatment groups.

FIGS. 34A-E. CD4+ TCR clonotype distribution in the mouse breast tumor samples and TCR-peptide binding prediction for the three TOP2A peptides. (FIG. 34A) Distribution of CD4+ TCR clonotypes in breast tumor tissues from TOP2A vaccine treated mice. (FIG. 34B) Distribution of CD4+ TCR clonotypes in the breast tumor tissues from non-treated CpG control mice. Predicted binding of the following peptides with the CD4+ TCR clonotypes detected in breast tumor tissues from the TOP2A vaccinated mice: (FIG. 34C) First TOP2A peptide-KDIVALMURRAYDIA (SEQ ID NO: 1); (FIG. 34D) Second TOP2A peptide-ILNWVKFKAQVQLNKK (SEQ ID NO: 2); (FIG. 34E) Third TOP2A peptide-KKWKVKYYKGLGTSTSK. (SEQ ID NO: 3)

FIGS. 35A-35F. Overexpression of the TOP2A gene in mouse and human TNBC tissues. (FIG. 35A) Overexpression of the TOP2A gene in M6 mouse mammary carcinoma cells vs. M28 normal mammary cells; both cell lines were derived from C3 (1)/Tag mice. (FIG. 35B) Overexpression of the TOP2A gene in human TNBCs. (FIG. 35C) Overexpression of the TOP2A gene in African American (AA) TNBCs. (FIG. 35D-35F) Immunohistochemistry of TOP2A expression on the mammary gland tissue of C3 (1)/Tag mice. (FIG. 35D) Expression in mammary gland tissue from a wild-type littermate of the C3 (1)/Tag mice. (FIG. 35E) Expression in ductal carcinoma in situ (DCIS). (FIG. 35F) Expression in invasive carcinoma. All images are scanned and captured by NanoZoomer system (Hamamatsu Photonics, Hamamatsu, Japan).

FIGS. 36A-36B. IFN-γ based ELISpot assay on C3 (1)/Tag mice. (FIG. 36A) Representative IFN-γ based ELISpot assay results showing T cell responses to specific TOP2A peptides from mouse splenocytes. (FIG. 36B) IFN-γ based ELISpot assay results. Splenocytes were collected from vaccinated mice and pulsed with negative control peptide (HIV peptide), positive control peptide (Concanavalin A), 3 individual TOP2A peptides, or their combination (combo). After 72 h of incubation, the ELISpot assay was performed, plates were scanned, and spot numbers were statistically analyzed. Data are shown as the mean±SE of three replicate wells per group, n=5, **, p<0.01****, p<0.0001 (2-tail t-test).

FIGS. 37A-37D. Clustering analysis of scRNA-seq data from TNBC and lymph node tissue immune cells sorted by flow cytometry. (FIG. 37A) The expression of the marker genes for the CD8+T, CD4+T, DNT, DC, macrophages and neutrophils in mouse TNBC tumor samples from control TOP2A vaccinated mice. (FIG. 37B) Landscape of the overall immune cell populations from the tumor samples of control and TOP2A vaccinated mice. (FIG. 37C) Expression of the marker genes for the CD8+T, CD4+T, DNT, DC and macrophages in the lymph node samples from control and TOP2A vaccinated mice. (FIG. 37D) Landscape of the immune cells in lymph nodes of control and TOP2A vaccinated mice.

FIGS. 38A-38D. Effects of TOP2A vaccine treatment on the DNT cells (CD4-CD8-double negative T cells) in breast tumor samples. (FIG. 38A) Canonical T cell marker expression was used to generate UMAP plots from scRNA-seq data. (FIG. 38B) Heatmap of the expression of the markers for each DNT cell subset. (FIG. 38C) Distribution of each DNT cell subset. (FIG. 38D) Percent changes of the DNT cell subsets across the control and the TOP2A vaccine treatment groups.

FIGS. 39A-39D. Effects of TOP2A vaccine treatment on DNT cell subsets in lymph node tissues. (FIG. 39A) Canonical marker expression on DNT cells was used to generate UMAP plots from scRNA-seq data. (FIG. 39B) Heatmap of markers expressed by each DNT cell subset. (FIG. 39C) UMAP plot distribution of each DNT cell subset. (FIG. 39D) Percent changes in the DNT cell subsets across the control and the TOP2A vaccine treatment groups.

FIGS. 40A-40D. Effects of the TOP2A vaccine on macrophages in the breast tumor samples. (FIG. 40A) Canonical markers expressed on macrophages were used to generate UMAP plots based on scRNA-seq data. (FIG. 40B) Heatmap based on the expression of the markers for each macrophage subset, M1 (anti-tumor) and M2 (pro-tumor) macrophages. (FIG. 40C) UMAP plot distribution of the M1 and M2 macrophage subsets. (FIG. 40D) Percent changes in the M1 and M2 macrophage subsets in control versus TOP2A vaccine-treated mice.

FIGS. 41A-41D. Effects of TOP2A vaccine treatment on macrophages in lymph nodes. (FIG. 41A) Canonical markers expressed on macrophages were used to generate UMAP plots based on scRNA-seq data. (FIG. 41B) Heatmap based on the expression of the markers for M1 and M2 macrophage subsets. (FIG. 41C) UMAP plot distribution of the M1 and M2 macrophage subsets. (FIG. 41D) Percent changes in the M1 and M2 macrophage subsets in control versus TOP2A vaccine-treated mice.

FIGS. 42A-42D. Effects of the TOP2A vaccine on dendritic cells (DC) in the breast tumor samples. (FIG. 42A) Canonical markers expressed on DC were used to generate UMAP plots based on scRNA-seq data. (FIG. 42B) Heatmap based on the expression of the markers for each DC subset, conventional dendritic cells (CDC) and plasmacytoid dendritic cells (pDC). (FIG. 42C) UMAP plot distribution of the cDC and pDC subsets. (FIG. 42D) Percent changes in the cDC and pDC subsets in control versus TOP2A vaccine-treated mice.

FIGS. 43A-43D. Effects of TOP2A vaccine treatment on DC in the lymph node samples. (FIG. 43A) Canonical markers expressed on DC were used to generate UMAP plots based on scRNA-seq data. (FIG. 43B) Heatmap based on the expression of the markers for cDC and pDC subsets. (FIG. 43C) UMAP plot distribution of the cDC and pDC subsets. (FIG. 43D) Percent changes in the cDC and pDC subsets in control versus TOP2A vaccine-treated mice.

FIGS. 44A-44D. Effects of TOP2A vaccine treatment on neutrophils in breast tumor samples. (FIG. 44A) Canonical markers expressed on neutrophils were used to generate UMAP plots based on scRNA-seq data. (FIG. 44B) Heatmap based on the expression of the markers for the neutrophil subsets, Stage I and Stage II. (FIG. 44C) UMAP plot distribution of Stage I and Stage II neutrophil subsets. (FIG. 44D) Percent changes in Stage I and Stage II subsets in control versus TOP2A vaccine-treated mice.

FIG. 45. Flow Gating Strategy used for flow cytometry in FIG. 31D-31I.

### DETAILED DESCRIPTION

### Overview

Approximately 20% of breast cancers are classified as triple negative breast cancer (TNBC) as they do not express estrogen receptor (ER), progesterone receptor (PR) and human epidermal growth factor receptor 2 (HER2), encompassing the molecular subtypes of basal-like and the more recently defined claudin-low group. TNBC is also associated with African American race, younger age, higher grade and mitotic index, and more advanced stage at diagnosis. Specific risk factors that correlate with TNBC include reproductive factors (pregnancy and multiple childbirths) and obesity. In TNBC patients, the 5-year survival rate is much lower than other forms of breast cancer including ER+PR+HER-(Luminal A) and HER+subtypes. Luminal A tumors have targeted therapies, e.g. hormonal agents (tamoxifen, aromatase inhibitors), whereas HER+ breast cancers are treated with antibodies or small molecule inhibitors. In contrast, early and intermediate stages of TNBC are routinely treated with standard cytotoxic chemotherapy resulting in strong initial regression in most patients. However, resistance develops in most patients. With the current challenges in treating TNBC, new approaches are needed to improve clinical outcomes.

Cancer vaccines to date have been primarily used for treating active, late-stage cancers, which provides limited efficacy presumably due to the immune suppression that is intrinsic to advanced malignancies. Immune suppression can involve multiple mediators including T regulatory cells (Treg), inhibitory macrophages and other suppressive factors. One way to potentially use cancer vaccines more effectively is in the prevention or early anti progression setting. Several reports have now provided proof-of-concept to support the use of peptide vaccines targeting overexpressed self-antigens as immunoprevention for breast cancer (Lollini et al., 2006; Disis et al., 2013; Ebben et al., 2015; Pan J et al., 2017). Molecular analysis of tumors has identified many genes that are overexpressed in breast cancer, which can be exploited as tumor antigens and potential vaccine candidates. Currently, the most common tumor antigens used in cancer immunotherapy are upregulated self-proteins, such as HER2. Vaccination with peptides targeting overexpressed HER2/neu in humans has been shown to be effective and well-tolerated (Schneble et al., 2014; Lowenfeld et al., 2016). While mutated epitopes are recognized as foreign “neo-antigens” by the immune system, eliciting a type 1 immune response, epitopes derived from non-mutated self-antigens are more likely to trigger T helper 2 (Th2) cytokines such as interleukin (IL)-10 and IL-6 that can inhibit cytotoxic T-lymphocyte (CTL) proliferation and function. Recently, attempts have been made to specifically identify Th1-selective epitopes from non-mutated self-antigens that can elicit a neo-antigen-like response. Th1-selective epitopes, when used in a vaccine, can elicit unopposed type 1 immunity and can be effective in preventing cancer growth in preclinical models. If Th2-inducing epitopes from the same protein are included in a vaccine, Th2 cells elicited by immunization may abrogate the Th1-mediated anti-tumor effect (Cecil et al., 2014; Disis et al., 1996). If the antigens are expressed early in oncogenesis, vaccines could have utility in prevention.

In the present disclosure, databases of The Cancer Genome Atlas (TCGA) were utilized in conjunction with transcriptomic and/or proteomic analysis of normal vs. malignant human breast tissues to identify highly expressed genes in the malignant tissues, and it was found that topoisomerase 2 alpha (Top2A) is highly expressed in human TNBC. Top2A is a known key enzyme in DNA replication, cancer cell proliferation and a target of several cytotoxic agents that directly or indirectly affect Top2A. Recent studies suggested that Top2A has a potential application in breast cancer detection and management (Klintman et al., 2016). As described herein, one or more polypeptides that may be utilized as a Top2A peptide vaccine were developed and its immunogenicity and preventive efficacy against TNBC in a mouse model was evaluated. As described below, single-cell RNA sequencing (scRNA-sq) analyses showed that a Top2A vaccine induced anti-tumor CD4+Th1 cells and cytotoxic CD8+ T cells in mouse breast tumor and lymph node tissue samples. Additionally, it was demonstrated that a Top2A vaccine induces a potent Top2A-specific memory immune response that prevents secondary challenge tumorigenesis. Finally, T-Cell Receptor (TCR) sequences from CD4 TIL cells in tumors from vaccinated mice were examined and found that there were TIL cells with TCR sequences against all of the immunizing peptides. In addition to breast cancer, TOP2A is highly overexpressed/amplified in many tumor types including lung cancer. As described herein, significant preventative effects were found in lung cancer mouse models that were vaccinated with the compositions disclosed herein. In light of the extensive and unexpected data, the compositions and/or polypeptides disclosed herein that may be utilized as a Top2A vaccine are highly immunogenic and efficacious in the treatment and/or prevention of cancer, including breast cancer such as TNBC, and lung cancer.

### Definitions and Terminology

The disclosed polypeptides, compositions, and methods for treating, or for providing preventative treatment, of one or more cancers, e.g., breast cancer and/or TNBC, may be further described using definitions and terminology as follows. The definitions and terminology used herein are for the purpose of describing particular aspects only and are not intended to be limiting.

As used in this specification and the claims, the singular forms “a,” “an,” and “the” include plural forms unless the context clearly dictates otherwise.

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean up to plus or minus 10% of the particular term and “substantially” and “significantly” will mean more than plus or minus 10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion of additional components other than the components recited in the claims. The term “consisting essentially of” should be interpreted to be partially closed and allowing the inclusion only of additional components that do not fundamentally alter the nature of the claimed subject matter.

The phrase “such as” should be interpreted as “for example, including.” Moreover, the use of any and all exemplary language, including but not limited to “such as”, is intended merely to better illuminate the invention and does not pose a limitation on the scope of the invention unless otherwise claimed.

Furthermore, in those instances where a convention analogous to “at least one of A, B and C, etc.” is used, in general such a construction is intended in the sense of one having ordinary skill in the art would understand the convention (e.g., “a system having at least one of A, B and C” would include but not be limited to systems that have A alone, B alone, C alone, A and B together, A and C together, B and C together, and/or A, B, and C together.). It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or “B” or “A and B.”

All language such as “up to,” “at least,” “greater than,” “less than,” and the like, include the number recited and refer to ranges which can subsequently be broken down into ranges and subranges. A range includes each individual member. Thus, for example, a group having 1-3 members refers to groups having 1, 2, or 3 members. Similarly, a group having 6 members refers to groups having 1, 2, 3, 4, or 6 members, and so forth.

The modal verb “may” refers to the preferred use or selection of one or more options or choices among the several described embodiments or features contained within the same. Where no options or choices are disclosed regarding a particular embodiment or feature contained in the same, the modal verb “may” refers to an affirmative act regarding how to make or use an aspect of a described embodiment or feature contained in the same, or a definitive decision to use a specific skill regarding a described embodiment or feature contained in the same. In this latter context, the modal verb “may” has the same meaning and connotation as the auxiliary verb “can.”

The terms “identical” or percent “identity,” in the context of two or more nucleic acids or polypeptide sequences, refer to two or more sequences or subsequences that are the same or have a specified percentage of amino acid residues or nucleotides that are the same (i.e., 50%, 55%, 60%, 65%, 70%, 75%, 80%, 85%, 90%, 91%, 92%, 93%, 94%, 95%, 96%, 97%, 98%, 99%, or more identity over a specified region, e.g., of an entire nucleic acid or polypeptide sequence or individual portions or domains of a nucleic acid or polypeptide), when compared and aligned for maximum correspondence over a comparison window, or designated region as measured using one of the following sequence comparison algorithms or by manual alignment and visual inspection. Such sequences are then said to be “substantially identical.” This definition also refers to the complement of a test sequence, in the context of nucleic acids. By way of example, in embodiments, the identify exists over a region that is about or at least about 5, 10, 15, 20, 50, 100, or 1000, amino acids in length, to about, less than about, or at least about 220, 100 or 1000 amino acids or nucleotides in length. Optionally, the identity exists over a region that is at least about 5, 10, 15, or 16 amino acids in length (e.g., with reference to SEQ ID NO: 1) to about 100, about 20 to about 75, about 30 to about 50 amino acids or nucleotides in length. Non-limiting examples of polypeptide sequences provided herein comprise sequences that are substantially identical to SEQ ID NO: 1, SEQ ID NO: 2, and/or SEQ ID NO: 3. By way of example, polypeptides that are at least 90%, 91%, 92%, 93%, 94%, 95%, 96%, 97%, 98%, 99% identical to SEQ ID NO: 1, SEQ ID NO: 2, and/or SEQ ID NO: 3 is provided herein. Polypeptides comprising a difference of 1, 2, 3, 4, 5, 6 or 7, 8, 9 or 0 amino acids as compared to SEQ ID NO: 1, SEQ ID NO: 2, and/or SEQ ID NO: 3 are also contemplated herein.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

An example of algorithms suitable for determining percent sequence identity and sequence similarity are the BLAST and BLAST 2.0 algorithms, which are described in Altschul et al., Nuc. Acids Res. 25:3389-3402 (1977) and Altschul et al., J. Mol. Biol. 215:403-410 (1990), respectively. As will be appreciated by one of skill in the art, the software for performing BLAST analyses is publicly available through the website of the National Center for Biotechnology Information (NCBI). In embodiments, BLAST and BLAST 2.0 are used, with the parameters described herein, to determine percent sequence identity for the nucleic acids and proteins. In embodiments, a BLAST algorithm involves first identifying high scoring sequence pairs (HSPs) by identifying short words of length W in the query sequence, which either match or satisfy some positive-valued threshold score T when aligned with a word of the same length in a database sequence. In embodiments, T is referred to as the neighborhood word score threshold (Altschul et al., supra). In embodiments, these initial neighborhood word hits act as seeds for initiating searches to find longer HSPs containing them. In embodiments, the word hits are extended in both directions along each sequence for as far as the cumulative alignment score can be increased. In embodiments, cumulative scores are calculated using, for nucleotide sequences, the parameters M (reward score for a pair of matching residues; always >0) and N (penalty score for mismatching residues; always <0). In embodiments, for amino acid sequences, a scoring matrix is used to calculate the cumulative score. In embodiments, extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. In embodiments, the BLAST algorithm parameters W, T, and X determine the sensitivity and speed of the alignment. In embodiments, the NCBI BLASTN or BLASTP program is used to align sequences. In embodiments, the BLASTN or BLASTP program uses the defaults used by the NCBI. In embodiments, the BLASTN program (for nucleotide sequences) uses as defaults: a word size (W) of 28; an expectation threshold (E) of 10; max matches in a query range set to 0; match/mismatch scores of 1, −2; linear gap costs; the filter for low complexity regions used; and mask for lookup table only used. In embodiments, the BLASTP program (for amino acid sequences) uses as defaults: a word size (W) of 3; an expectation threshold (E) of 10; max matches in a query range set to 0; the BLOSUM62 matrix (see Henikoff & Henikoff, Proc. Natl. Acad. Sci. USA 89:10915 (1992)); gap costs of existence: 11 and extension: 1; and conditional compositional score matrix adjustment.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, Y-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence with respect to the expression product, but not with respect to actual probe sequences.

As to amino acid sequences, one of skill will recognize that individual substitutions to a peptide, polypeptide, or protein sequence which alters a single amino acid is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles.

The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V): 6) Phenylalanine (F), Tyrosine (Y), Tryptophan (W); 7) Serine(S), Threonine (T); and 8) Cysteine (C), Methionine (M) (see, e.g., Creighton, Proteins (1984)).

“Breast cancer” as used herein refers to a malignant cancer of cells of the breast.

“Triple negative breast cancer (TNBC)” as used herein refers to breast cancer that does not express estrogen receptor (ER), progesterone receptor (PR), and human epidermal growth factor receptor 2 (HER2). TNBC may encompass the molecular subtypes of basal-like and the claudin-low group.

“Lung cancer” as used herein refers to a malignant cancer of cells of the lungs.

The term “subject” may be used interchangeably with the terms “individual” and “patient” and includes human and non-human subjects. In some embodiments, subjects may be any mammal.

As used herein, the terms “treat” or “treatment” encompass both “preventative” and “curative” treatment. “Preventative” treatment is meant to indicate a postponement of development of a disease, a symptom of a disease, or medical condition, suppressing symptoms that may appear, or reducing the risk of developing or recurrence of a disease or symptom. “Curative” treatment includes reducing the severity of or suppressing the worsening of an existing disease, symptom, or condition. Thus, treatment includes ameliorating or preventing the worsening of existing disease symptoms, preventing additional symptoms from occurring, ameliorating or preventing the underlying systemic causes of symptoms, inhibiting the disorder or disease, e.g., arresting the development of the disorder or disease, relieving the disorder or disease, causing regression of the disorder or disease, relieving a condition caused by the disease or disorder, or stopping the symptoms of the disease or disorder.

A non-limiting example of a treatment for a subject includes, but is not limited to, administering to a subject a composition comprising one or more polypeptides, where the one or more polypeptides comprise or consist of one or more of: a polypeptide that is at least 80%, 85%, 90%, 95%, or 99% identical to SEQ ID NO: 1; a polypeptide that is at least 80%, 85%, 90%, 95%, or 99% identical to SEQ ID NO: 2; or a polypeptide that is at least 80%, 85%, 90%, 95%, or 99% identical to SEQ ID NO: 3. In one or more aspects, the one or more polypeptides comprise or consist of one or more of a polypeptide having the sequence of SEQ ID NO: 1, a polypeptide having the sequence of SEQ ID NO: 2, or a polypeptide having the sequence of SEQ ID NO: 3. In various aspects, the composition may also include an adjuvant. Another non-limiting example of a treatment for a subject includes, but is not limited to, administering to a subject a composition comprising a polynucleotide encoding for one or more of: a polypeptide that is at least 80%, 85%, 90%, 95%, or 99% identical to SEQ ID NO: 1; a polypeptide that is at least 80%, 85%, 90%, 95%, or 99% identical to SEQ ID NO: 2; or a polypeptide that is at least 80%, 85%, 90%, 95%, or 99% identical to SEQ ID NO: 3. In various aspects, the polynucleotide can be mRNA.

A non-limiting example of a preventative treatment for a subject includes, but is not limited to, administering to a subject a composition comprising one or more polypeptides, where the one or more polypeptides comprise or consist of one or more of: a polypeptide that is at least 80%, 85%, 90%, 95%, or 99% identical to SEQ ID NO: 1; a polypeptide that is at least 80%, 85%, 90%, 95%, or 99% identical to SEQ ID NO: 2; or a polypeptide that is at least 80%, 85%, 90%, 95%, or 99% identical to SEQ ID NO: 3. In one or more aspects, the one or more polypeptides comprise or consist of one or more of a polypeptide having the sequence of SEQ ID NO: 1, a polypeptide having the sequence of SEQ ID NO: 2, or a polypeptide having the sequence of SEQ ID NO: 3. In various aspects, the composition may also include an adjuvant. Another non-limiting example of a preventative treatment for a subject includes, but is not limited to, administering to a subject a composition comprising a polynucleotide encoding for one or more of: a polypeptide that is at least 80%, 85%, 90%, 95%, or 99% identical to SEQ ID NO: 1; a polypeptide that is at least 80%, 85%, 90%, 95%, or 99% identical to SEQ ID NO: 2; or a polypeptide that is at least 80%, 85%, 90%, 95%, or 99% identical to SEQ ID NO: 3. In various aspects, the polynucleotide can be mRNA.

As used herein, the term “adjuvant” refers to ingredients used in some vaccines to create a stronger immune response in subjects receiving the vaccine. By way of example, but not by way of limitation, adjuvants can include one or more of: oligonucleotides comprising CpG dinucleotides (CpG oligo deoxynucleotide), Montanide, aluminum, monophosphoryl lipid A (MPL), MPL+ aluminum salt, oil in water emulsion, e.g., composed of squalene, MPL and QS-21.

As used herein, “therapeutically effective amount” refers to an amount of a therapeutic agent, e.g., one or more of the compositions described herein, effective to treat a disease or disorder and/or effective to prevent a disease or disorder. For instance, a therapeutically effective amount of one or more of the compositions described herein may be an amount effective to elicit an immune response from a subject, such as for example, by stimulating local and/or systemic antigen-specific CD4+ and CD8+ T cell responses which may also produce an increase in one or more of IFN-γ, TNF-α, IL-2, or IL-23. Additionally or alternatively, as an example, a therapeutically effective amount of one or more of the compositions described herein may be an amount effective to reduce the number of cancer cells, reduce tumor size, inhibit or slow tumor growth and/or metastasis, and/or prevent occurrence of specific cancer cells, such as breast cancer cells, including TNBC cells.

### Compositions

In certain aspects, disclosed herein are one or more polypeptides comprising or consisting of fragments of topoisomerase 2 alpha (Top2A). In various aspects, the one or more polypeptides comprise or consist of one or more of: a polypeptide that is at least 80%, 85%, 90%, 95%, or 99% identical to SEQ ID NO: 1; a polypeptide that is at least 80%, 85%, 90%, 95%, or 99% identical to SEQ ID NO: 2; or a polypeptide that is at least 80%, 85%, 90%, 95%, or 99% identical to SEQ ID NO: 3. In one or more aspects, the one or more polypeptides comprise or consist of one or more of a polypeptide having the sequence of SEQ ID NO: 1, a polypeptide having the sequence of SEQ ID NO: 2, or a polypeptide having the sequence of SEQ ID NO: 3.

In various aspects, the compositions can alternatively or additionally include one or more of the polypeptides disclosed below in Table 1, and/or have a sequence that is at least 80%, 85%, 90%, 95%, or 99% identical to one or more of the sequences in Table 1.

TABLE 1
Polypeptides

| SEQ ID NO: | Peptide Sequence |
|---|---|
| SEQ ID NO: 1 | KDIVALMVRRAYDIA |
| SEQ ID NO: 2 | ILNWVKFKAQVQLNKK |
| SEQ ID NO: 3 | KKWKVKYYKGLGTSTSK |
| SEQ ID NO: 4 | ISIWNNGKGIPVVEH |
| SEQ ID NO: 6 | SILNWVKFKAQVQLNKK |
| SEQ ID NO: 8 | IIPMVLINGAEGIGT |
| SEQ ID NO: 9 | AERVGLHKVFKLQTSLT |

In certain aspects, the compositions can alternatively or additionally can comprise or consist of one or more of the polypeptides that are at least 80%, 85%, 90%, 95%, 99%, or 100% identical to one or more of SEQ ID NOs: 1-3, 4, 6, 8, 9, 20-27, and 28-30.

In various aspects, the compositions can include one or more fragments of topoisomerase 2 alpha (Top2A) and one or more other antigens, such as fragments of cyclin E2 and/or KIF15. In such aspects, the compositions can comprise or consist of one or more polypeptides that are at least 80%, 85%, 90%, 95%, 99%, or 100% identical to one or more of SEQ ID NOs: 1-3, 4, 6, 8, 9; and one or more polypeptides that are at least 80%, 85%, 90%, 95%, 99%, or 100% identical to one or more of SEQ ID NOs: 28-30. In various aspects, the fragments of topoisomerase 2 alpha (Top2A) and one or more other antigens, such as fragments of cyclin E2 and/or KIF15 can be present in one continuous polypeptide and may or may not be separated by a linker.

In various aspects, the compositions can include one or more MHC-II epitopes of Top2A and one or more MHC-I epitopes of Top2A. For example, in various aspects, the compositions can comprise or consist of one or more polypeptides that are at least 80%, 85%, 90%, 95%, 99%, or 100% identical to one or more of SEQ ID NOs: 1-3, 4, 6, 8, 9; and one or more polypeptides that are at least 80%, 85%, 90%, 95%, 99%, or 100% identical to one or more of SEQ ID NOs: 20-27. In various aspects, the MHC-II epitope(s) and the MHC-I epitope(s) can be present in one continuous polypeptide and may or may not be separated by a linker.

In various aspects, the compositions can include a polypeptide having more than one copy of a polypeptide sequence disclosed herein, or at least 80%, 85%, 90%, 95%, 99% of a polypeptide sequence disclosed herein. In various aspects, the copies may or may not be separated by a linker. For instance, the polypeptide can be at least 80%, 85%, 90%, 95%, 99%, or 100% identical to SEQ ID NO: 31, in one aspect.

In various aspects, the composition can be a vaccine or a component of a vaccine.

In certain aspects, the composition can be a vaccine or a component of a vaccine that includes any form of vaccine that can provide one or more of the epitopes exhibited by the one or more polypeptides described herein. For instance, in one aspect, the vaccine or component of a vaccine can include a polynucleotide, mRNA, and/or DNA that encodes for one or more of the polypeptides disclosed herein and that is configured to be expressed in the subject. In the same or alternative aspects, the vaccine or component of a vaccine can include a polynucleotide, mRNA, and/or DNA that encodes for one or more of: a polypeptide that is at least 80%, 85%, 90%, 95%, 99%, or 100% identical to SEQ ID NO: 1; a polypeptide that is at least 80%, 85%, 90%, 95%, 99%, or 100% identical to SEQ ID NO: 2; or a polypeptide that is at least 80%, 85%, 90%, 95%, 99%, or 100% identical to SEQ ID NO: 3. In various aspects, the vaccine or component of a vaccine can include a polynucleotide, mRNA, and/or DNA that encodes for a polypeptide that is at least 80%, 85%, 90%, 95%, 99%, or 100% identical to SEQ ID NO: 31.

In various aspects, when the composition comprises a polynucleotide encoding for one or more of the polypeptide sequences disclosed herein, or at least 80%, 85%, 90%, 95%, 99% of one or more of the polypeptide sequences disclosed herein, the polynucleotide can be one or more mRNAs. In certain aspects, the one or more mRNAs or polynucleotide can be present in any suitable vehicle for administration to a subject. In one aspect, the one or more mRNAs or polynucleotide can be present in a lipid nanoparticle (LNP).

In one or more aspects, the composition can include, or optionally include, an adjuvant. In various aspects, the adjuvant can include any adjuvant that is suitable for use in a vaccine. In certain aspects, the adjuvant can include one or more of CpG dinucleotides (CpG oligo deoxynucleotide), Montanide, aluminum, monophosphoryl lipid A (MPL), MPL+ aluminum salt, oil in water emulsion, e.g., composed of squalene, MPL and QS-21.

### Methods

In various aspects, methods are disclosed for treating a subject and/or preventative treatment of a subject for a cancer, including breast cancer and/or lung cancer.

In various aspects, the methods for the treatment of a subject and/or preventative treatment of a subject are for the treatment and/or preventative treatment for a breast cancer, including TNBC.

In various aspects, the methods for the treatment of a subject and/or preventative treatment of a subject are for the treatment and/or preventative treatment for lung cancer.

In various aspects, the methods can include administering to a subject one or more of the compositions disclosed herein.

In one or more aspects, the methods disclosed herein include administering a vaccine to a subject, where the vaccine comprises one or more of the compositions disclosed herein.

As discussed above, in various aspects, the compositions disclosed for use in the methods herein can include one or more polypeptides. In certain example aspects, the one or more polypeptides can comprise or consist of one or more of: a polypeptide that is at least 80%, 85%, 90%, 95%, or 99% identical to SEQ ID NO: 1; a polypeptide that is at least 80%, 85%, 90%, 95%, or 99% identical to SEQ ID NO: 2; or a polypeptide that is at least 80%, 85%, 90%, 95%, or 99% identical to SEQ ID NO: 3. In one or more aspects, the one or more polypeptides comprise or consist of a polypeptide having the sequence of SEQ ID NO: 1, a polypeptide having the sequence of SEQ ID NO: 2, and/or a polypeptide having the sequence of SEQ ID NO: 3. In one or more aspects, the composition can include, or optionally include, an adjuvant. Additional example polypeptides and polynucleotides are described above and referenced herein.

In some aspects, the subject has not previously been diagnosed with breast cancer and/or TNBC. In the same or alternative aspects, the subject has not been treated for breast cancer and/or TNBC. In various aspects, the subject has been previously diagnosed with breast cancer and/or TNBC. In one or more aspects, the breast cancer does not express one or more of: an estrogen receptor (ER), a progesterone receptor (PR), or human epidermal growth factor 2 (HER2). In various aspects, the subject exhibits one or more risk factors associated with TNBC selected from pregnancy, multiple child births, and obesity.

In certain aspects, the subject has not previously been diagnosed with lung cancer. In the same or alternative aspects, the subject has not been treated for lung cancer. In various aspects, the subject has been previously diagnosed with lung cancer. In one or more aspects, the subject exhibits one or more risk factors associated with lung cancer, including but not limited to, smoking or being a prior smoker.

In certain aspects, at least one, or at least two doses of a therapeutically effective amount of the composition is administered to the subject according the methods described herein. In certain aspects, one or more doses of a therapeutically effective amount of the composition may be administered over a desired time interval, e.g., monthly, yearly, every two, three, four, five, or ten years.

In various aspects, in the methods described herein, administering the composition to a subject may result in an increased level of one or more of IFN-γ, TNF-α, IL-2, or IL-23 in the subject compared to an untreated control. In various aspects, an untreated control can be another subject and/or healthy person that did not receive the composition and/or a subject that received a placebo. In certain aspects, in the methods described herein, administering the composition to a subject may result in stimulating local and/or systemic antigen-specific CD4+ and CD8+ T cell responses compared to an untreated control. In one or more aspects, in the methods described herein, administering the composition to a subject may result in reducing, slowing, or inhibiting tumor growth, metastasis, and/or development, e.g., breast cancer tumor growth, metastasis, and/or development, including TNBC, compared to an untreated control. In the same or alternative aspects, in the methods described herein, administering the composition to a subject may result in reducing one or more symptoms associated with breast cancer, including TNBC.

In certain aspects, the methods disclosed herein contemplate combination treatments. For instance, in one or more aspects, the methods disclosed herein may include administering one or more of the compositions described herein to a subject that is already undergoing or concurrently undergoing treatment for a cancer, e.g., breast cancer, including TNBC. In various aspects, the subject that is already or concurrently undergoing treatment may be receiving one or more cytotoxic chemotherapeutic agents. In certain aspects, a subject undergoing such a combination therapy may experience an increased reduction in symptoms associated with cancer and/or a further reduction, slowing, or inhibition of cancer growth, metastasis, or development compared to another subject that is not undergoing combination therapy (e.g., not receiving the compositions described herein but is receiving other treatments for cancer).

### EXAMPLES

The following Examples are illustrative and are not intended to limit the scope of the claimed subject matter.

Triple-negative breast cancer (TNBC) is an aggressive subtype of breast cancer, comprising approximately 20% of all breast cancers, that has a poor prognosis due to its aggressive behavior and lack of effective targeted therapies. In the present study, in silico analyses were utilized to identify preferentially expressed genes in TNBC and found that topoisomerase 2 alpha (Top2A) is highly expressed both at transcriptional and protein levels. Top2A is a key enzyme in DNA replication and is a therapeutic target for breast and other cancers. Overexpression of Top2A was confirmed in human breast cancer tissue microarrays and mouse TNBCs derived from C3 (1)/Tag mice. Here, Top2A-specific MHC II epitopes with optimal binding affinity were identified using a combined scoring system, which predicted their potential to elicit a Th1 immune response. Furthermore, all peptides were selected to have identical amino acid sequence between humans and mice for potential clinical translation. Of the candidate Top2A peptides examined, three peptides were selected based on strong IFN-γ ELISPOT responses following immunization of tumor naïve mice and Top2A peptide-specific tetramer staining. The selected peptides were combined to form a multi-peptide Top2A vaccine. Splenocytes collected from Top2A peptide-vaccinated animals showed a robust immunologic response to the immunizing peptides, and in vitro stimulation of splenocytes with Top2A peptides increased the secretion of Th1 cytokines. Anti-tumor efficacy of the Top2A vaccine was demonstrated in a syngeneic TNBC mouse model [C3 (1)/Tag-REAR and M6 cells], in which pre-graft preventive vaccination was associated with significantly decreased tumor growth as compared to the adjuvant controls. In a genetically engineered mouse (GEM) model (C3 (1)/Tag), the vaccine was >90% effective in preventing tumor development. There were no overt toxicities observed with the Top2A vaccination. Finally, TCR sequences in CD4 TIL from vaccinated mice were examined and found that the TIL contained TCR sequences specific to all of the immunizing peptides. These data indicate that the newly developed multi-peptide Top2A vaccine is highly immunogenic, elicits TILs with the peptide-specific TCR, and is highly effective in preventing and intercepting TNBC development and progression in vivo.

Breast cancer is the leading malignancy in women with 281,550 estimated new cases and 43,600 estimated deaths reported in 2021 in the United States (Siege et al., 2021). Approximately 20% of breast cancers are classified as triple negative breast cancer (TNBC) as they do not express estrogen receptor (ER), progesterone receptor (PR) and human epidermal growth factor receptor 2 (HER2), encompassing the molecular subtypes of basal-like and the more recently defined claudin-low group (Prat et al., 2010). TNBC is also associated with African American race, younger age, higher grade and mitotic index, and more advanced stage at diagnosis (Jemal et al., 2011). Specific risk factors that correlate with TNBC include reproductive factors (pregnancy and multiple childbirths) and obesity. In TNBC patients, the 5-year survival rate is much lower than other forms of breast cancer including ER+PR+HER-(Luminal A) and HER+subtypes (Onitilo et al., 2009; Harbeck et al., 2017). Luminal A tumors have targeted therapies, e.g. hormonal agents (tamoxifen, aromatase inhibitors), whereas HER+ breast cancers are treated with antibodies or small molecule inhibitors (Gao and Swain, 2018). In contrast, early and intermediate stages of TNBC are routinely treated with standard cytotoxic chemotherapy resulting in strong initial regression in most patients. However, resistance develops in most patients. With the current challenges in treating TNBC, new approaches are needed to improve clinical outcomes.

Cancer vaccines to date have been primarily used for treating active, late-stage cancers, which provides limited efficacy presumably due to the immune suppression that is intrinsic to advanced malignancies. Immune suppression can involve multiple mediators including T regulatory cells (Treg), inhibitory macrophages and other suppressive factors. One way to potentially use cancer vaccines more effectively is in the prevention or early anti progression setting. Several reports have now provided proof-of-concept to support the use of peptide vaccines targeting overexpressed self-antigens as immunoprevention for breast cancer (Lollini et al., 2006; Disis et al., 2013; Ebben et al., 2015; Pan J et al., 2017). Molecular analysis of tumors has identified many genes that are overexpressed in breast cancer, which can be exploited as tumor antigens and potential vaccine candidates. Currently, the most common tumor antigens used in cancer immunotherapy are upregulated self-proteins, such as HER2. Vaccination with peptides targeting overexpressed HER2/neu in humans has been shown to be effective and well-tolerated (Schneble et al., 2014; Lowenfeld et al., 2016). While mutated epitopes are recognized as foreign “neo-antigens” by the immune system, eliciting a type 1 immune response, epitopes derived from non-mutated self-antigens are more likely to trigger T helper 2 (Th2) cytokines such as interleukin (IL)-10 and IL-6 that can inhibit cytotoxic T-lymphocyte (CTL) proliferation and function. Recently, attempts have been made to specifically identify Th1-selective epitopes from non-mutated self-antigens that can elicit a neo-antigen-like response. Th1-selective epitopes, when used in a vaccine, can elicit unopposed type 1 immunity and can be effective in preventing cancer growth in preclinical models. If Th2-inducing epitopes from the same protein are included in a vaccine, Th2 cells elicited by immunization may abrogate the Th1-mediated anti-tumor effect (Cecil et al., 2014; Disis et al., 1996). If the antigens are expressed early in oncogenesis, vaccines could have utility in prevention.

In the current study, databases of The Cancer Genome Atlas (TCGA) were used in conjunction with transcriptomic and/or proteomic analysis of normal vs. malignant human breast tissues to identify highly expressed genes in the malignant tissues, and it was found that topoisomerase 2 alpha (Top2A) is highly expressed in human TNBC. Top2A is a known key enzyme in DNA replication, cancer cell proliferation and a target of several cytotoxic agents that directly or indirectly affect Top2A. Recent studies suggested that Top2A has a potential application in breast cancer detection and management (Klintman et al., 2016). A Top2A multi-peptide vaccine was developed herein and evaluated for its immunogenicity and preventive efficacy against TNBC in a mouse model. Single-cell RNA sequencing (scRNA-sq) analyses showed that Top2A multi-peptide vaccination induced anti-tumor CD4+Th1 cells and cytotoxic CD8+ T cells in mouse breast tumor and lymph node tissue samples. Additionally, the Top2A vaccine induces a potent Top2A-specific memory immune response that prevents secondary challenge tumorigenesis. Finally, TCR sequences from CD4 TIL cells in tumors from vaccinated mice were examined and found that there were TIL cells with TCR sequences against all of the immunizing peptides. Taken together, the data demonstrate that the multi-peptide Top2A vaccine is highly immunogenic and efficacious in the prevention of TNBC.

### Materials and Methods

Mice. Transgenic C3 (1)/Tag mice and C3 (1)/Tag-REAR (abbreviation for rearrangement) mice were a generous gift from Dr. Jeffery E. Green. FVB/N wild-type mice were bought from the Jackson Laboratory. For all experiments, only F2 C3 (1)/Tag or C3 (1)/Tag-REAR generation mice were used. Mice were maintained and bred in the Biomedical Resource Center at the Medical College of Wisconsin (MCW), Milwaukee, WI. All procedures were approved by the Institutional Animal Care and Use Committee (IACUC).

Cell lines. M27 (weakly tumorigenic/benign tumor), M6 (malignant tumor), and M6C (metastatic tumor) and M28 (normal control) cells, all derived from C3 (1)/Tag mice, were provided by Dr. Jeffery E. Green (Holzer et al., 2003). The cell lines were maintained in DMEM medium containing high glucose (Gibco), supplemented with 5% FBS, penicillin/streptomycin and sodium pyruvate (Invitrogen).

Immunohistochemistry (IHC). IHC staining was performed by the Children's Research Institute Histology Core at MCW. Leica Bond Immunostainer Max (model #10897664) and Leica Bond Immunostainer RX system (model #11784892) were used for human tissue microarray (TMA) and murine samples, respectively. Mouse mammary gland samples from C3 (1)/Tag or C3 (1)/Tag-REAR mice were inflated, formalin-fixed and paraffin-embedded (Sakura Tissue Tek VIP5). Four μm sections were used for IHC or hematoxylin and eosin (H&E) staining. The numbers of CD4+ and CD8+ tumor-infiltrating T-lymphocytes (TIL) were determined as cells per mm2 tumor area by using CD4 antibody (Invitrogen 14-9766-82) and CD8 antibody (Invitrogen 14-0808-82). Entire slides were scanned using the NanoZoomer slide scanner (Hamamatsu). Subsequently, NanoZoomer software was used and tumor regions were specifically highlighted and measured.

Human tissue microarray analysis. Two types of human TMAs were purchased from US Biomax, Inc. (http://www.biomax.us/tissue-arrays/Breast/BRC961 and http://www.biomax.us/tissue-arrays/Breast/BRC962). Top2A antibody was purchased from Invitrogen, PAS-26255. Top2A expression on the human TMAs was assessed by using the scoring system: Score 0=no stain; Score 1=light stained; Score 2=medium stained; and Score 3=strong stained. When evaluating the tissue staining intensity for each group, scores of only 0 and I were translated as weak; scores of 0, 1 and less than 10%; 2 or 3 were translated as mild; and a score of 3 in more than 10% of samples in a given group was translated as high expression.

ELISPOT assay. Cell suspensions from whole spleens were filtered through a 70 μm cell strainer (BD) and subjected to red blood cell lysis using ACK lysis buffer. 1.5-3.0×10⁴ cells were plated into individual wells of a MAIPS4510 multiscreen 96-well plate coated with anti-interferon γ (IFN-γ) detection antibody and containing media with either peptide, concanavalin A (positive control), HIV peptide (negative control), or no antigen (negative control). After 72-hour incubation, plates were washed and incubated with a secondary antibody (BD) overnight at 4° C. Wells were then washed with PBS and HRP streptavidin was added. Following one-hour incubation, the plate was developed using AEC substrate for between five to 25 minutes. An automated plate reader system (CTL Technologies) was used to image the plates and quantify spot numbers.

Scoring system for the prediction of MHC class II binding epitopes. A combined scoring system was used to identify selected antigen-specific MHC epitopes with optimal binding affinity. The method was published by Dr. Disis and colleagues (Park et al., 2008). Briefly, to identify antigen-specific MHC class II epitopes that have optimal binding affinity and promiscuity across multiple alleles, the following algorithms were used for prediction: NetMHCIIpan (https://services.healthtech.dtu.dk/service.php?NetMHCIIpan-4.0, Technical University of Denmark, Lyngby, Denmark) and Rankpep (http://imed.med.ucm.es/Tools/rankpep.html, University Computense Madrid, Harvard, Madrid, Spain). For each available MHC class II allele, 20 peptide sequences were initially selected solely based on the rank-order of the predicted binding affinity from each algorithm. The sequences are approximately 15 amino acids in length. Individual amino acids for each selected peptide were assigned a score, with one being an amino acid contained in a peptide sequence that ranked highest for predictive binding affinity. Scoring individual amino acids accounted for the multiple-peptides overlaps occurring within and across algorithms. The scores(S) for each amino acid were summed up across the multiple MHC class II alleles from two algorithms. Then, the number (N) of MHC class II alleles, for which each amino acid was predicted to have high-affinity binding, was counted. The final score for each amino acid was calculated by multiplying S and N. For ease of identifying the most potentially immunogenic segments of the protein, each amino acid was assigned a color (from dark red to light blue) based on its final score percentile, with dark red being highest at >75% and light blue the lowest at <10%. The color strata are as follows: dark red ≥75% of highest score; red=50~75% of highest score; orange=40~50% of highest score; yellow=30~40% of highest score; green=20~30% of highest score; blue≤20% of highest score. Thus, the dark red color corresponds to a sequence where multiple peptides scored highly within an algorithm as well as across algorithms. Light blue represents sequences that are the least potentially immunogenic of all predicted high-binding peptides.

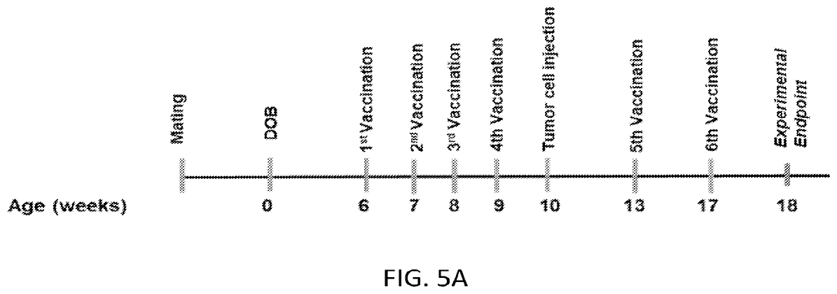

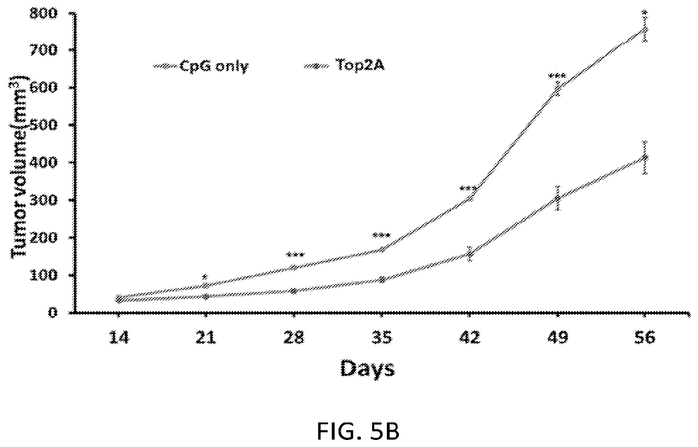

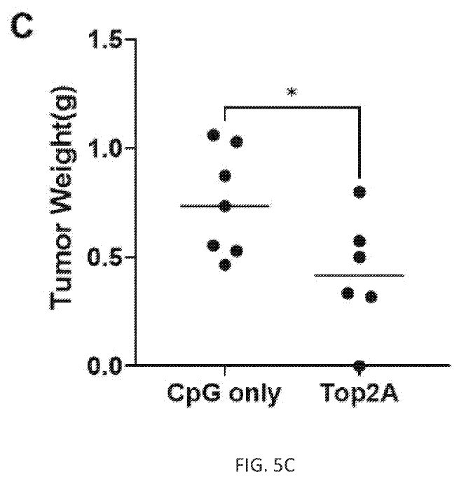

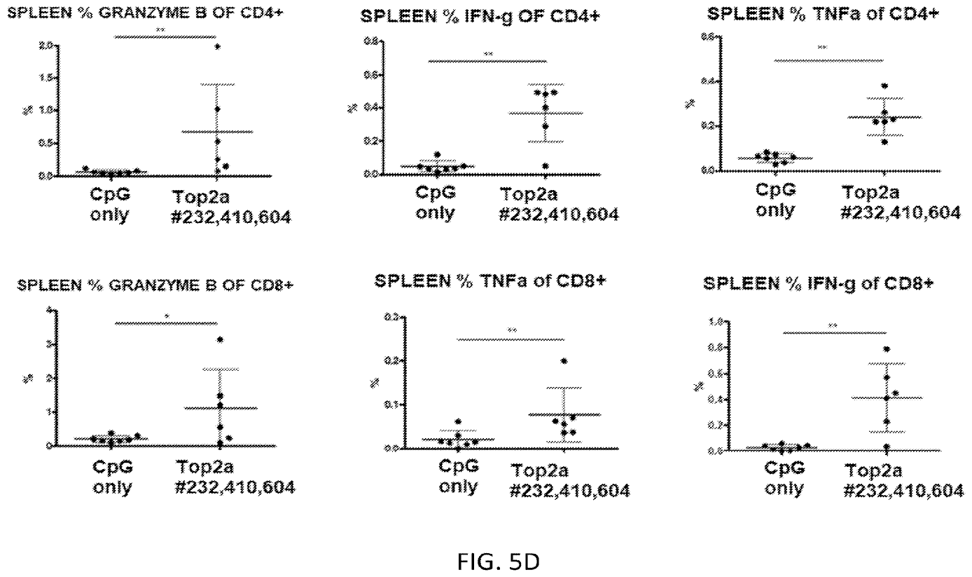

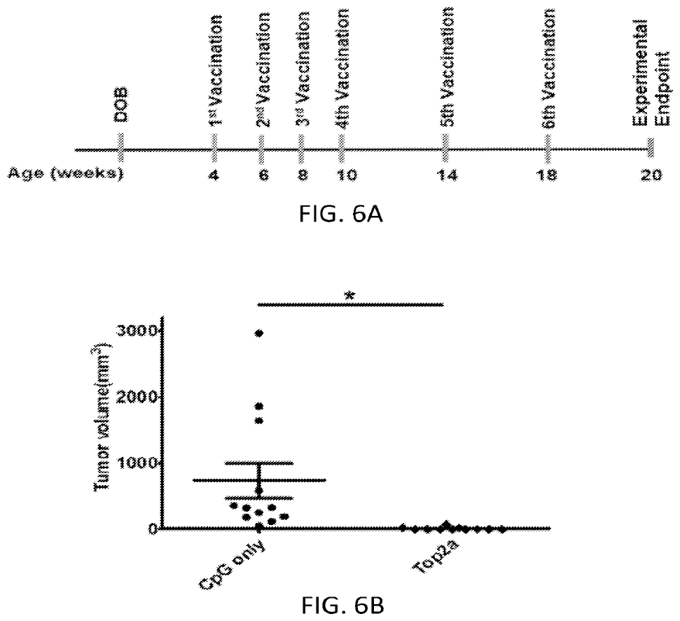

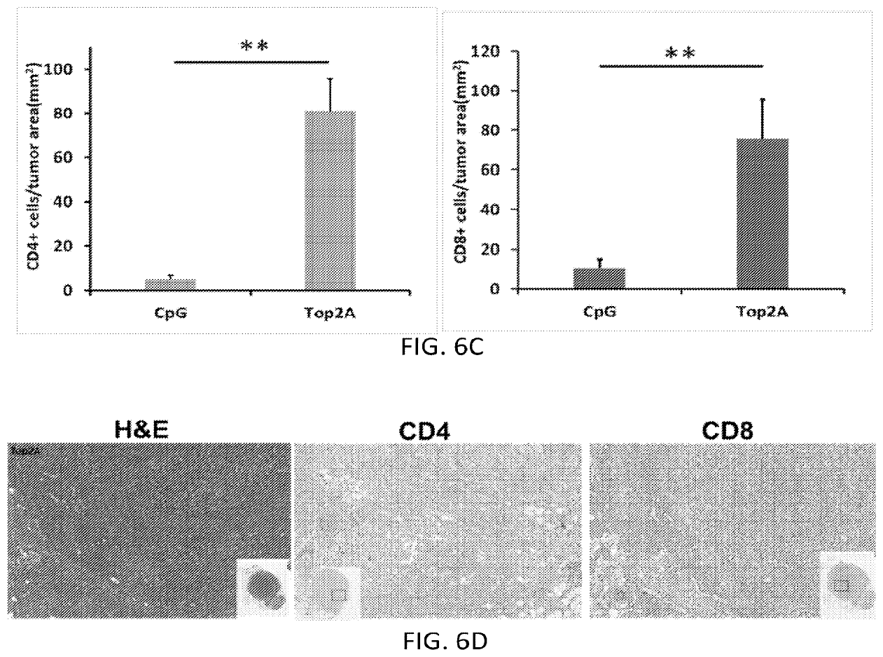

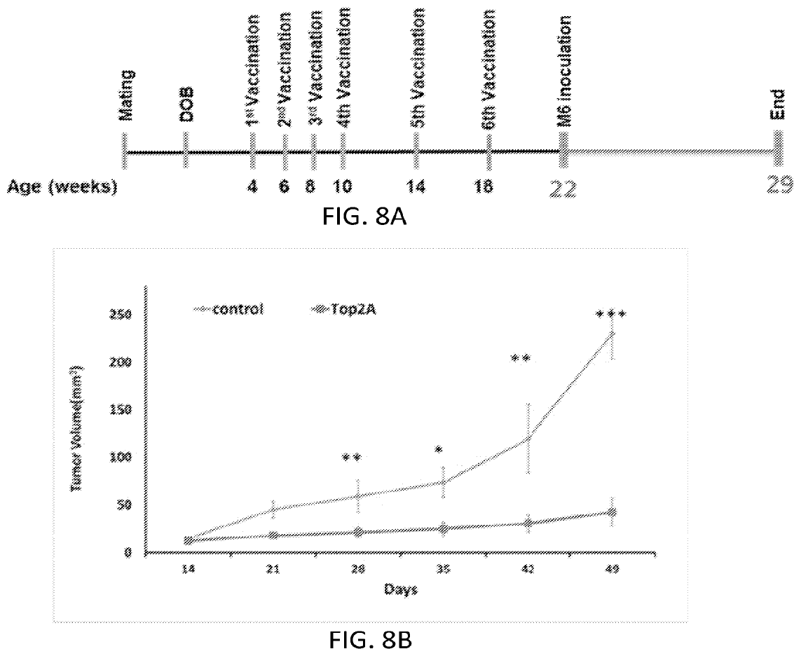

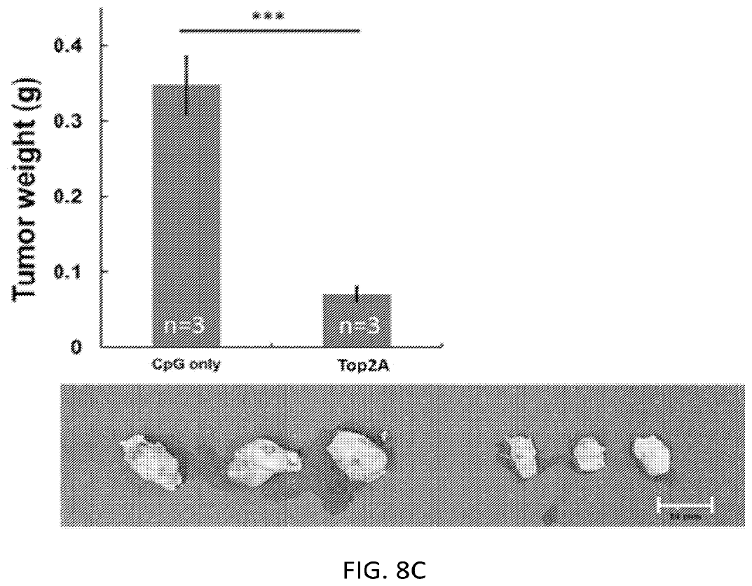

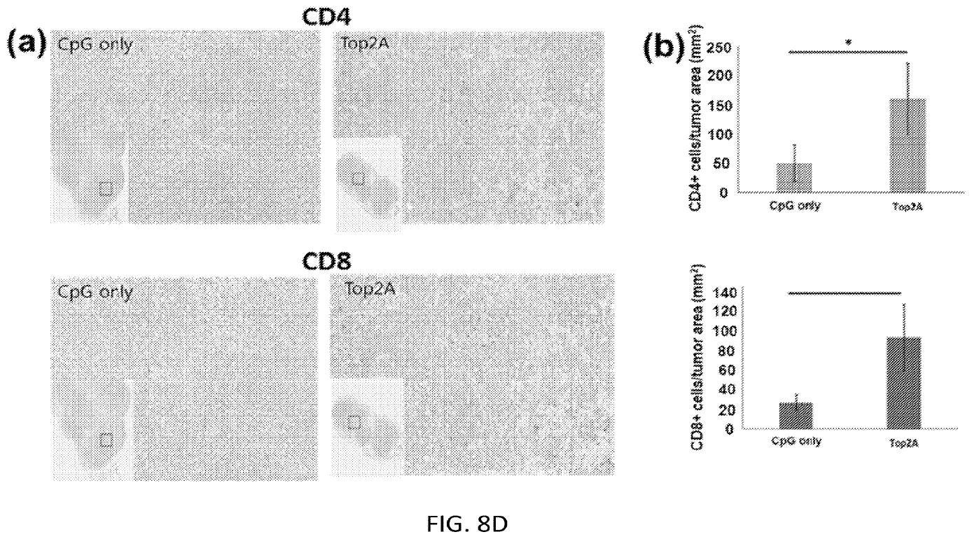

Vaccine preparation and immunization. Mice were vaccinated with 50 μg of each peptide. Three different Top2A peptides were purchased from Genemed Synthesis and diluted in phosphate-buffered saline (PBS) to 50 μl/mouse. Peptides and equal amounts of adjuvant CpG (Class B CpG oligonucleotide; a murine TLR9 ligand, Cat. No. tlrl-1826, InvivoGen) were added to bring the total vaccine volume to 100 μl/mouse. CpG was used at 50 μg per mouse. Mice were injected subcutaneously with Top2A vaccines following the timelines shown in FIGS. 5A and 8A.

Generation of dendritic cells. Murine DCs were generated from bone marrow stem cells as described previously (Li et al. 2021). Briefly, bone marrow cells were cultured at a density of 2×10⁵ cells/mL in 6-well plates, in RPMI-1640 complete medium supplemented with 20 ng/ml GM-CSF (R&D Systems, Minneapolis, MN). On day 4, the medium was replaced with fresh medium containing 10 ng/mL GM-CSF. On day 8, immature DCs were collected, pooled, and pulsed with Top2A peptides at a concentration of 50 μg/ml. TNF-α (10 ng/mL) and IL-1B (10 ng/mL) (R&D Systems) were added, and after 48 hours of culture, mature DCs were collected and used.

Tetramer assay. Top2A-specific T cells were generated from splenocytes of Top2A vaccinated FVB/N wild-type mice by repeated stimulations of autologous T cells with Top2A peptide-loaded mature DCs. In brief, isolated CD4+ T cells (5×105/500 μL/well) were cocultured with Top2A peptide-loaded mature DCs (1×105/500 μL/well) in 24 well plates at 37° C. in 5% CO₂ for 7 to 10 days in T-media (RPMI1640, 10% FBS, 1% Penicillin/streptomycin, 1×β-ME) including 10 ng/ml of IL-2. After culture, T cells were collected and stained with 3 types of Top2A specific tetramers (synthesized by NIH Tetramer Core Facility at Emory University, Atlanta, GA).

CD4+ T cell proliferation assay. Isolated CD4+ T cells were labeled with 5 (6)-carboxyfluorescein diacetate succinimidyl ester (CFSE; 5 μM; Invitrogen) for 10 minutes at 37° C., washed and then seeded (5×105/500 μL/well) into 24 well plate in T-media including 10 ng/ml of IL-2. Mature DCs (1×105/500 μL/well) pulsed with Top2A peptides were added to the plates and cultured for 4 days at 37° C. in 5% CO2. Flow cytometry analysis was used to detect the dilution of CFSE to trace cell proliferation. Results are expressed as mean counts per minute of triplicate cultures.

In vivo tumorigenicity assay. In the syngeneic model, C3 (1)/Tag-REAR mice were generated from a C3 (1)/Tag founder line through the loss of the original multiple copies of the C3 (1)/Tag-antigen transgene (Aprelikova et al., 2016). M6 cells, derived from a C3 (1)/Tag transgenic mammary tumor, were implanted into the mammary fat pad of C3 (1)/Tag-REAR mice. M6 cells were washed, resuspended in PBS at a density of 1×10⁶ cells in 100 μl PBS, and injected into the #4 mammary fat pads of female C3 (1)/Tag-REAR mice. Following implantation, tumor diameters were measured using calipers and tumor volumes were calculated using the formula: maximum diameter×(minimum diameter)²×0.4. In the spontaneous GEM model, C3 (1)/Tag mice were treated with the Top2A peptide vaccine following the experimental design in FIG. 8A. C3 (1)/Tag mice were sacrificed at 20-weeks of age for estimation of tumor development. Tumor volumes were measured using calipers and calculated using the formula: maximum diameter×(minimum diameter)²×0.4.

Cytokine analysis. Mouse Th1/Th2/Th17 Cytokines Multi-AnalyteELISArray™ Kits (Qiagen) were used for cytokine analysis. The cytokines represented by this array are IL2, IL4, IL5, IL6, IL10, IL12, IL13, IL17A, IL23, IFN-γ, TNFα, and TGFβ1. Splenocytes from different groups of mice were stimulated with different peptides for 72 hours, and then culture supernatants were collected and assayed based on the manufacturer's instructions.

Flow cytometry. Cell pellets were incubated with surface markers of interest at the recommended or titrated concentrations, incubated at 40C for 30 minutes, and protected from light. After incubation, cells were washed and resuspended in FACS fixation buffer for either analysis or intracellular staining. To begin intracellular staining, cells were fixed with Foxp3/Transcription factor staining buffer set (eBioscience) and stained with intracellular markers of interest at the recommended or titrated concentrations at 46C for at least 30 minutes while protecting them from light. Samples were washed with permeabilization buffer and resuspended in FACS fixation buffer. Stained cells were fixed in 1% paraformaldehyde and permeabilized following the manufacturer's instructions to evaluate the expression of intracellular targets, granzyme B, IFN-γ and TNF-α. Flow cytometry was conducted using an LSR-II flow cytometer (BD). Data were analyzed using FlowJo software (Tree Star).

Western blotting. Cells were lysed with 200 μl of 1× NP40 lysis buffer containing proteinase inhibitor cocktails (Thermo-Fisher), incubated for 20 minutes on ice, centrifuged at 16,000 g for 30 minutes, then the protein concentration was normalized as determined by the BCA method (Fisher Scientific, Pittsburgh, PA) and subsequently boiled for five minutes. The normalized lysate was resolved by 4-12% sodium dodecyl sulfate-polyacrylamide gel electrophoresis (SDS-PAGE) (Thermo-Fisher) and immunoblotted with Top2A antibody (PA5-26255, Thermo-Fisher) and GAPDH (sc-25778, Santa Cruz).

Single-cell RNA sequencing (scRNAseq) and TCR sequencing (scTCRseq). For single-cell sequencing, lymph node samples (1 sample from CpG control group and 2 samples from TOP2A vaccine treated group) and the breast tumor samples (1 sample from CpG control group and 1 sample from TOP2A vaccine treated group) of C3 (1)/Tag mice were harvested at the end of the study, minced and digested at 37.C for 30 minutes with mouse tumor dissociation buffer (Miltenyi Biotec, CA) to generate single-cell suspensions per the manufacturer's instructions. The processed samples were directly stained with violet viability dye, APC anti-CD45, and CD45+ leukocytes were sorted using FACS. FACS sorted CD45+ leukocytes were then spun down at 300 g for 5 minutes and counted manually with a Neubauer Chamber. Approximately 2.0×10⁴ cells were loaded onto the 10× Chromium Controller per the manufacturer's instruction, resulting in a recovery of about 1×10⁴ cells. For the lymph node samples, the libraries of single-cell transcriptome were generated by Chromium Single Cell 3′ v3 Reagent Kits (10× Genomics). For tumor samples, single-cell transcriptome and single-cell TCR libraries were prepared using a 10× Chromium Single-cell 5′ and VDJ library construction kit. All of the libraries were sequenced using NextSeq 500/550 High Output Kits v2 (150 cycles) (Illumina) according to the manufacturer's protocol.

scRNA-seq data analysis. Raw sequencing data were de-multiplexed and converted to gene-barcode matrices using the Cell Ranger (version 2.2.0) mkfastq and count functions, respectively (10× Genomics). The mouse reference genome mm10 was used for alignment. Data were further analyzed in R (version 3.4.0) using Seurat (version 3). The number of genes detected per cell, number of unique molecular identifiers (UMIs), and the percent mitochondrial genes were plotted, and outliers were removed to filter out doublets and dead cells. Raw UMI counts were normalized and log transformed. Integrated analysis was then performed to identify shared cell clusters that are present across different datasets. Principal component analysis was performed using variable genes, and the top 20 most statistically significant principal components were used for UMAP analysis.

Statistical analysis. All in vitro assays were performed at least in triplicate. Six to twelve mice per group were used for the in vivo studies. A two-tailed Student's t-test was used to evaluate differences between the control group and each treatment group. P-values<0.05 were considered statistically significant.

### Example 1—Gene Expression Analyses of Top2A

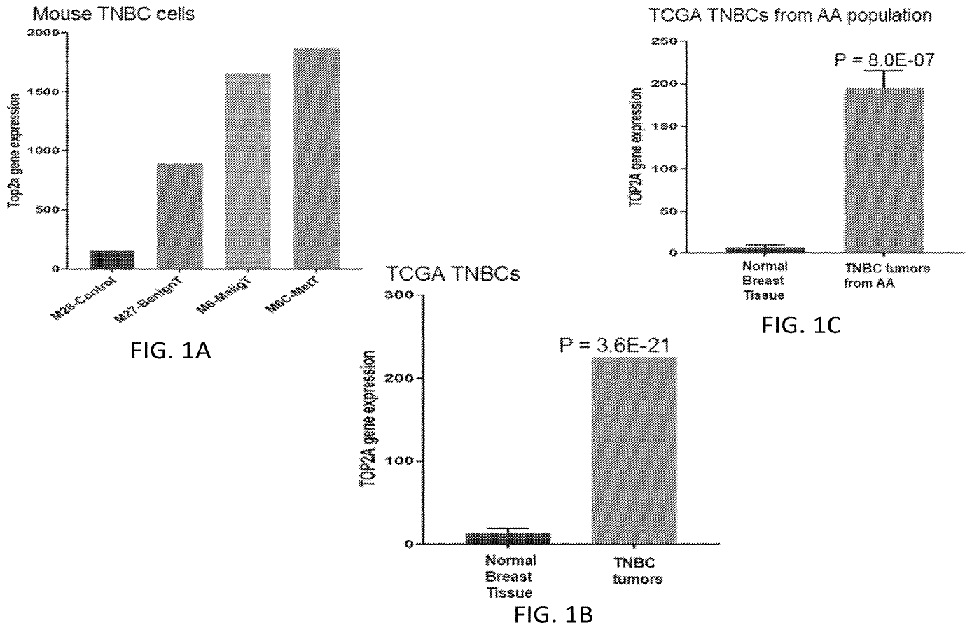

It was found that the Top2A gene was overexpressed in various mouse breast tumor cell lines and human TNBC and lung tumors (FIG. 1). In mice, the Top2a gene was overexpressed in mouse mammary tumor cell lines M27 (mammary benign tumor cell line), M6 (mammary malignant tumor cell line), and M6C (mammary metastatic tumor cell line) cell lines, with the M28 mammary cell line serving as a normal control (FIG. 1A). The Top2A gene was also highly overexpressed in TCGA TNBC samples (12 normal breast tissues and 137 tumor samples of the TNBC patients) with limited expression in normal mammary tissues (FIG. 1B). We also performed expression analysis of TNBC in the African American (AA) population, which is at high risk for TNBC. Similar to the general TNBC population, Top2A was significantly overexpressed in TNBC from the AA subpopulation (FIG. 1C). These data indicate that the Top2A gene is overexpressed in TNBCs isolated from both mice and humans.

### Example 2—Increased Top2A Protein Expression in Human and Mouse TNBC Tissues

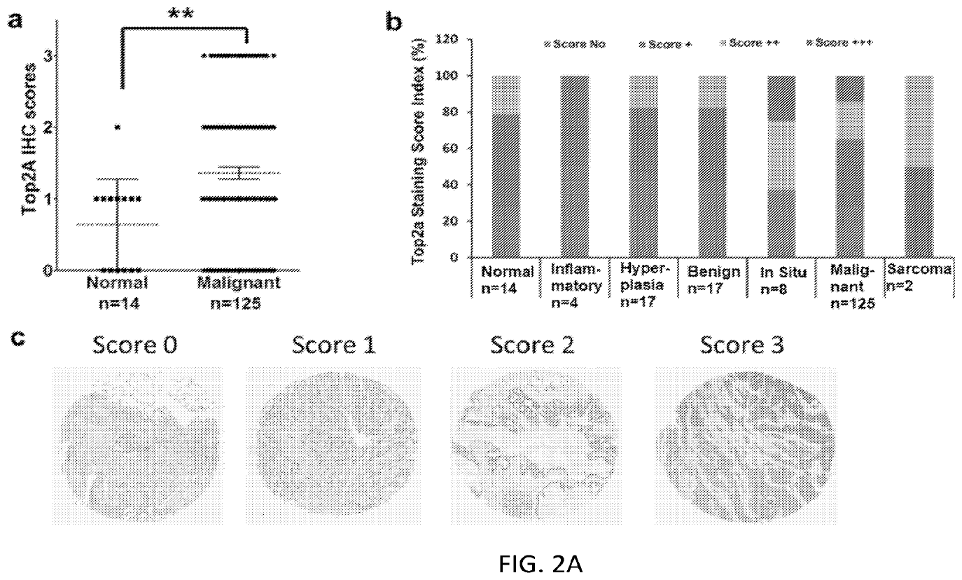

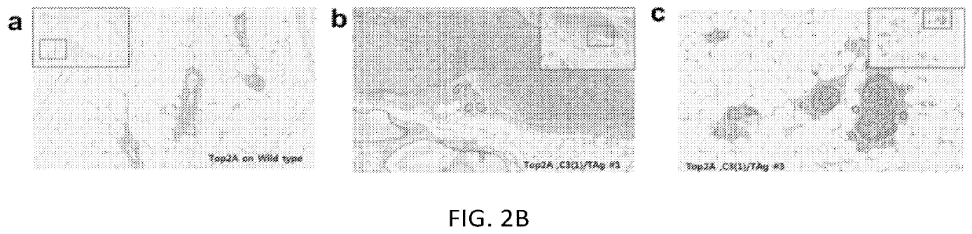

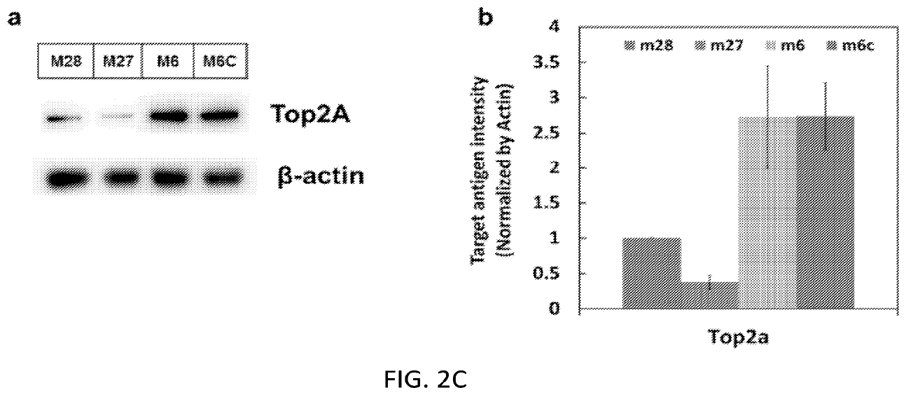

Using human breast carcinoma Tissue MicroArrays (TMA, BRC961 and 962, US Biomax, Inc), it was validated that Top2A is highly expressed in human TNBC. The TMAs included 36 cases of common carcinoma types (including TNBC) and 12 cases of normal and non-malignant breast tissues. Top2A protein expression levels were evaluated using the scoring system shown in FIG. 2A, and it was found that Top2A was overexpressed in human malignant tissues (FIG. 2A). In mice, mammary tissue samples, including tumors and corresponding normal mammary glands from C3 (1)/Tag female mice and FVB/N wild-type mice at 19-weeks of age, were harvested and analyzed by IHC. Representative images of Top2A IHC staining are shown in FIG. 2B, and Top2A was highly expressed on ductal carcinoma in situ (DCIS) and malignant tumor tissue from C3 (1)/Tag mice. In contrast, Top2A expression was not detected in normal mammary tissue from wild-type mice. The overexpression of Top2A was confirmed in murine breast cancer cell lines M28, M27, M6, and M6C as shown in FIG. 2C.

### Example 3-Identification of Th1 Top2A Epitopes for Vaccine Design

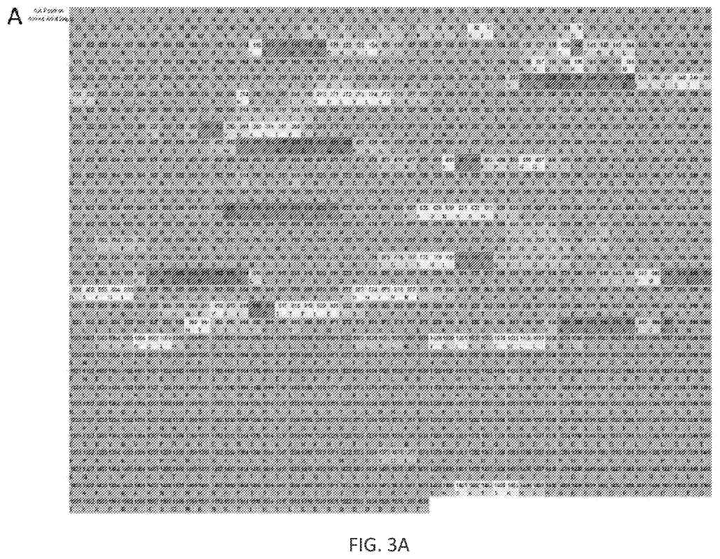

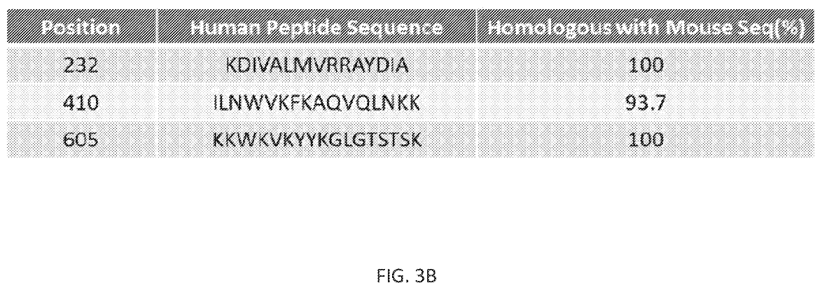

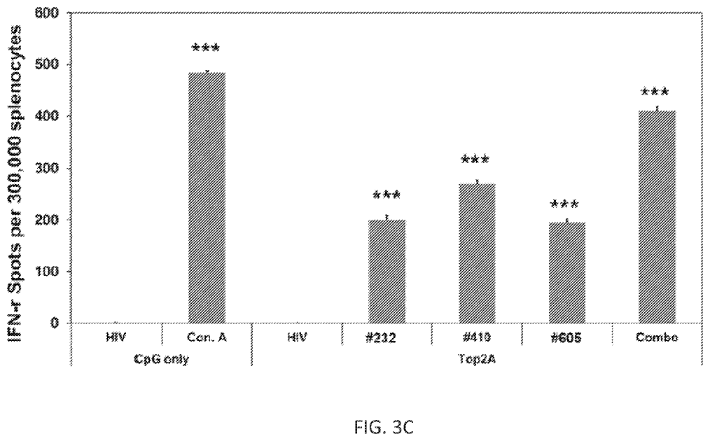

Using a multi-scoring system that combines multiple MHC class II peptide binding algorithms, 3 candidate peptides, #232 (p232-p246), #410 (p410-p425), and #604 (p605-p621) (FIG. 3A,B) were identified and selected. The immunogenicity of Top2A peptides was evaluated in C3 (1)/Tag-REAR mice via IFN-γ ELISPOT assays (FIG. 3C). Splenocytes from Top2A vaccinated demonstrated strong immune response with a mean for IFN-γ secreting cells of around 400 spots per well (SPW) compared to less than 10 SPW for negative control HIV peptides. Additionally, the immunogenicity of all three peptides combined (combo) was examined. Unexpectedly, combo vaccinated mice yielded a significantly stronger immune response than single peptide vaccinated mice (FIG. 3C). Sequences of the three peptides were 100% homologous between human and mouse Top2A. Peptides #232, #410, and #604 were chosen to formulate a multi-peptide Top2A vaccine for the preventive efficacy study.

### Example 4—In Vivo Immunogenicity of Top2A Peptides

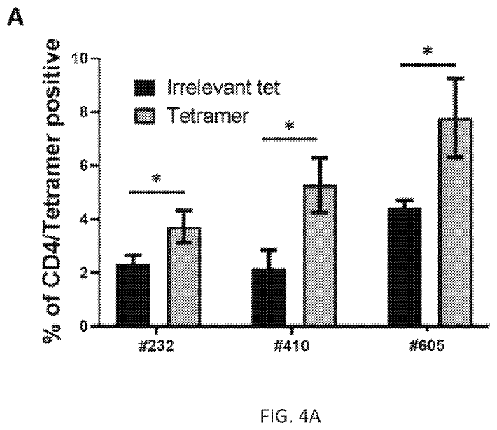

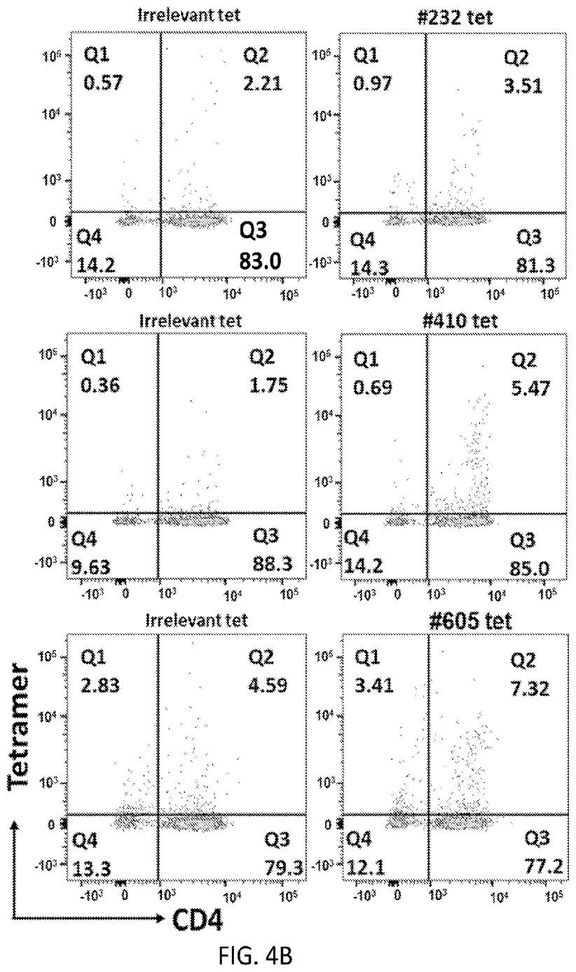

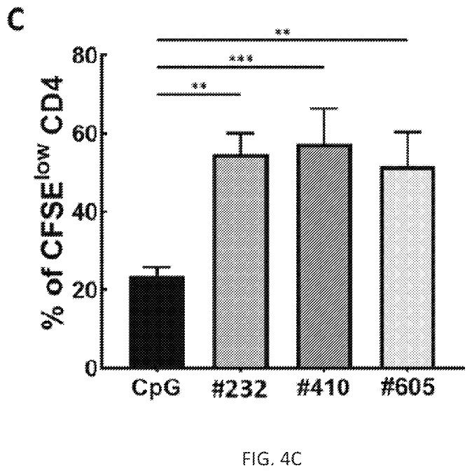

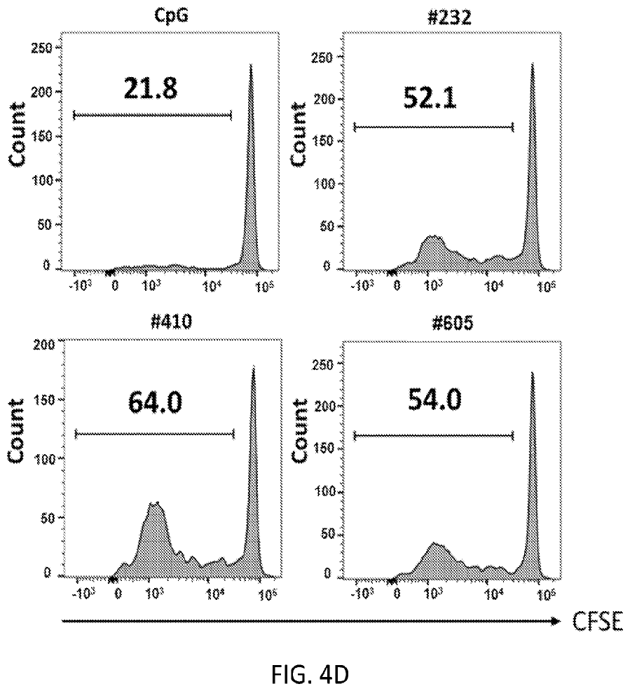

To examine whether the Top2A peptides could induce peptide-specific CD4+ T cells, FVB/N wild-type mice (3 mice per peptide) were subcutaneously injected with 50 μg Top2A peptides in CpG adjuvant. One week after the 4th immunization, splenocytes were collected, CD4+ T cells isolated, the cells re-stimulated with peptide-pulsed DCs for 4-7 days, and then analyzed for peptide reactivity. In vivo immunization successfully generated peptide-specific CD4+ T cells, as detected by flow cytometry with Top2A-peptide tetramers (FIG. 4 A,B). Furthermore, CD4 T cells from peptide immunized mice displayed significantly higher cell proliferation against Top2A peptide-pulsed murine DCs than CD4 T cells from the CpG only group (FIG. 4 C,D). These results indicate that the Top2A peptides were able to induce a peptide-specific T cell response in FVB/N wild-type mice

### Example 5—Top2A Multi-Peptide Vaccination Inhibited Development of Tumors in the Syngeneic TNBC Mouse Model

Experiments were designed in a syngeneic TNBC mouse model, C3 (1)/Tag-REAR mice inoculated with M6 tumor cells. As illustrated in FIG. 5A, 7-week old C3 (1)/Tag-REAR mice were given an initial vaccination of peptides with CpG oligodeoxynucleotides (CpG ODN) adjuvant, followed by three more vaccinations at one-week intervals. One week after the last vaccination, M6 cells were implanted into #4 mammary fat pads of the C3 (1)/Tag-REAR mice. Additional vaccinations were administered at four-week intervals until the end of the experiments. Notably, Top2A vaccination significantly decreased tumor volumes as compared to CpG-only treated control mice (FIG. 5A; p<0.05). Average tumor size at the experimental endpoint was 757.2 mm³ in control vs. 413 mm³ in vaccinated animals. Top2A vaccine also reduced tumor weight by nearly 40% (FIG. 5C, p<0.05). Top2A vaccinated mice were also examined for presence of a systemic immune response. Splenocytes collected from Top2A vaccinated mice were stained for the intracellular markers (granzyme B, IFN-γ and TNFα) and analyzed by flow cytometry. Significant increases in CD4+ and CD8+ T cells expressing granzyme B, IFN-γ, and TNFα were observed in the spleens of Top2A vaccinated animals as compared to controls treated with CpG only (FIG. 5D). Taken together, these results indicate that the Top2A peptide vaccine induces both systemic and local immune responses.

### Example 6—Top2A Vaccination Induced a Th1 Cytokine Response

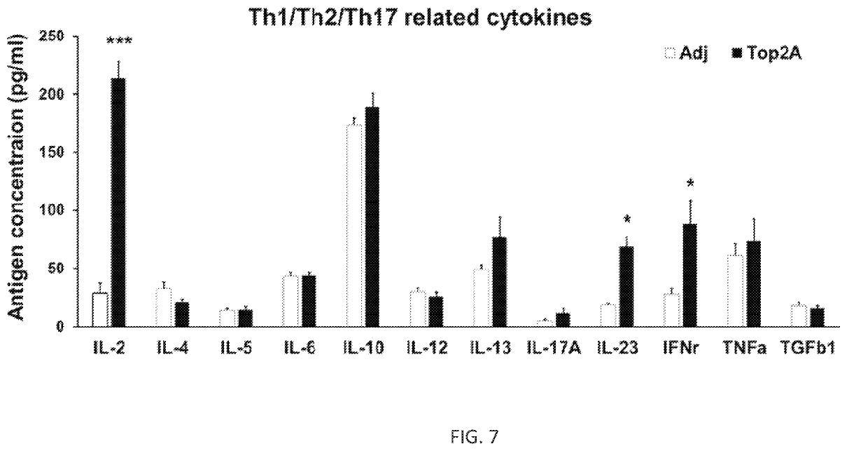

To evaluate Th1 and Th2 cytokine production in response to the Top2A vaccine, the production of 12 cytokines was measured from in vitro peptide-stimulated splenocytes that had been isolated from vaccinated mice (FIG. 7). The most abundant cytokines detected in response to the Top2A peptide pool were Th 1 cytokines IL-2 and IFN-γ, increasing approximately 7- and 3-fold, respectively, as compared to adjuvant controls. In contrast, Th2 cytokine production (IL-4, IL-5 and IL-13) did not increase as compared to the adjuvant controls. Interestingly, the Top2A vaccine stimulated not only Th 1 cytokine production (IL-2 and IFN-γ) but also IL-23 which is known to be produced by antigen presenting cells. These data suggest that a Th1 immune response is predominantly elicited by the Top2A-specific peptide vaccine.

### Example 7—Top2A Vaccinated C3 (1)/Tag Mice were Protected from the M6 Cell Secondary Challenge

Experiments were designed to test long-term immune memory protective effects of the Top2A vaccine against secondary tumor cell development. The first “challenge” was the spontaneous tumor that develops in C3/Tag mice (see FIG. 8A for experimental design). Secondary tumor challenge involved transplanting the C3/Tag mice at 22 weeks of age (4 weeks after the final vaccine) with syngeneic M6 tumor cells. Notably, Top2A vaccinated mice did not develop spontaneous tumors, and the vaccinated mice had significantly smaller M6 TNBC tumors at the experimental end point (FIG. 8B,C). Average tumor size in the vaccinated mice as compared to the adjuvant controls was 42 mm³ vs. 229.9 mm3, respectively (FIG. 8B; p<0.001). In Top2A vaccinated mice, Tumor weight in the Top2A vaccinated mice was also significantly reduced by nearly 85% (FIG. 8C). The numbers of CD4+ and CD8+ TILs in Top2A vaccinated mice were also significantly increased, as shown in FIG. 8D. These results suggest that the Top2A vaccine induces long-term T cell memory to help resist tumor re-emergence.

### Example 8—Single Cell Gene Expression Landscape in Breast Tumor and Lymph Node Tissues

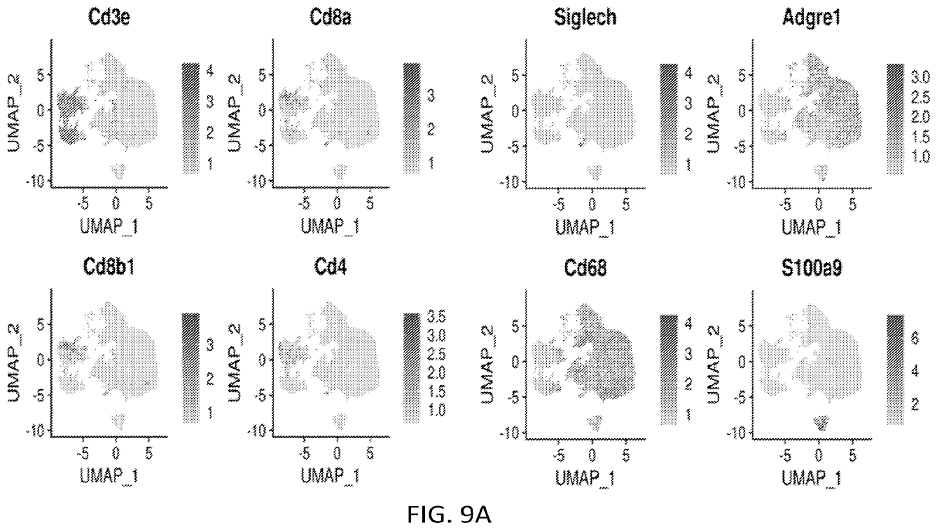

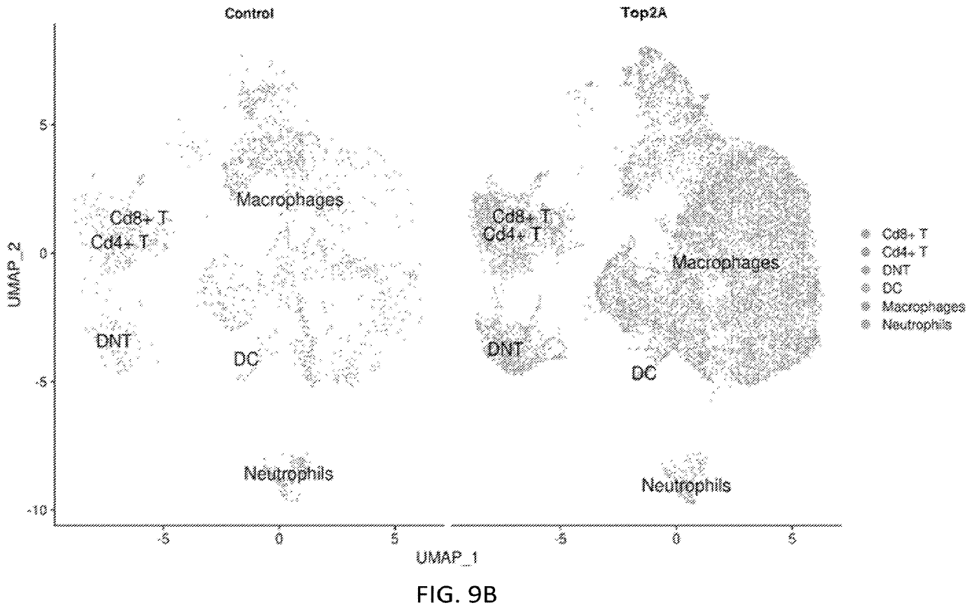

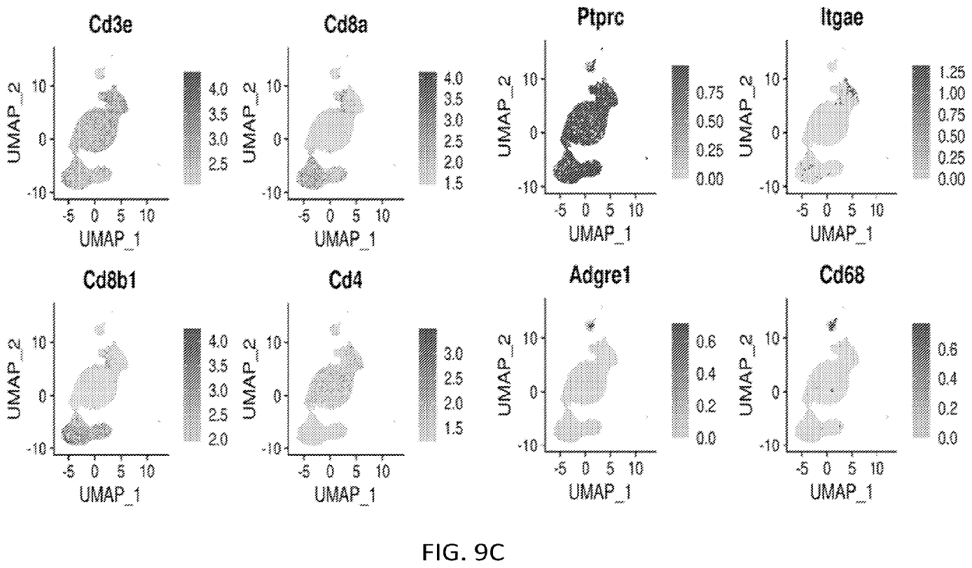

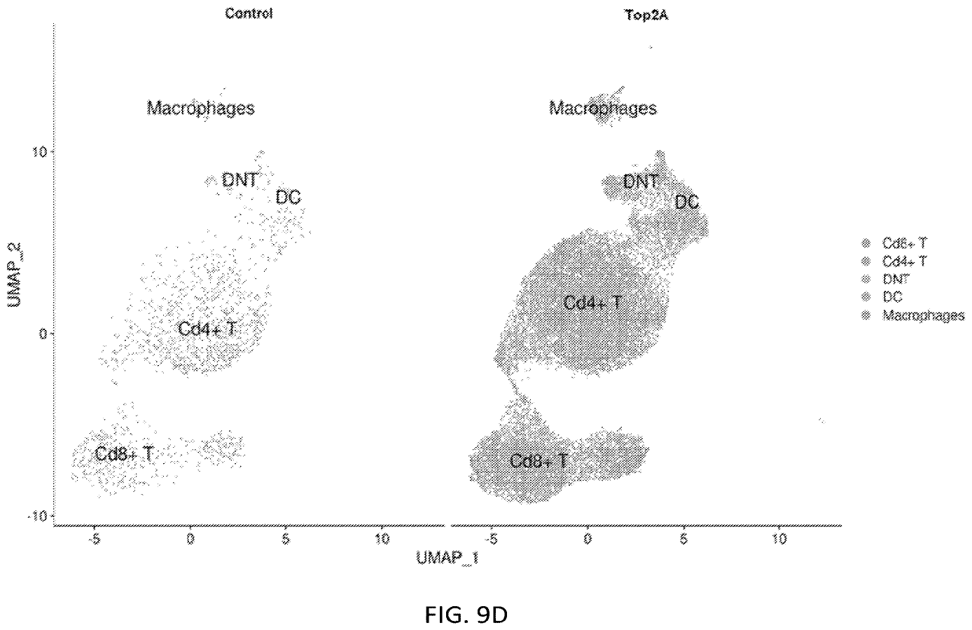

The Seurat package was utilized (Butler et al., 2018; Stuart et al., 2019) to perform fine clustering of sequenced single cells from mouse breast tumor and lymph node tissues. Gene expression data of single cells was aligned and projected in 2-dimensions using uniform manifold approximation and projection (UMAP) (Bech et al., 2018). The gene expression patterns of canonical markers were analyzed to characterize different types of immune cell populations in the tumor samples. Six immune cell populations were detected in the breast tumor samples including CD8+ T cells, CD4+ T cells, CD4/CD8 double-negative T cells (DNT), dendritic cells (DC), macrophages and neutrophils (FIG. 9A,B). Single cell expression data from the lymph node tissues was also projected by UMAP and immune cell populations identified included CD8+ T cells, CD4+ T cells, DNT, DC and macrophages, as expected (FIG. 9C,D).

### Example 9—Top2A Vaccine Treatment Increased the Proportions of Tumor-Specific Cytotoxic CD8+ T Cells in Mouse Breast Tumor and Lymph Node Tissues

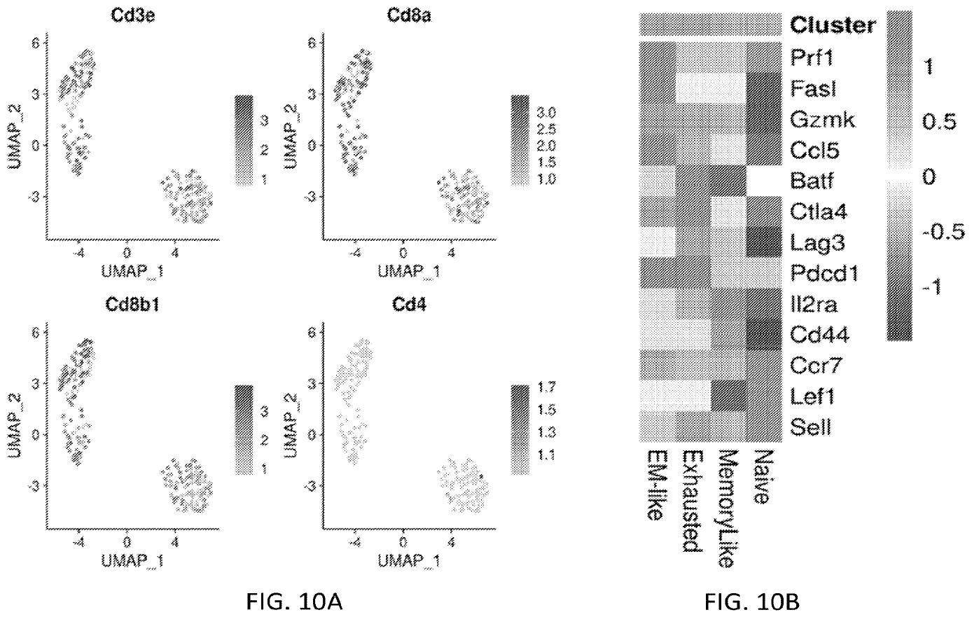

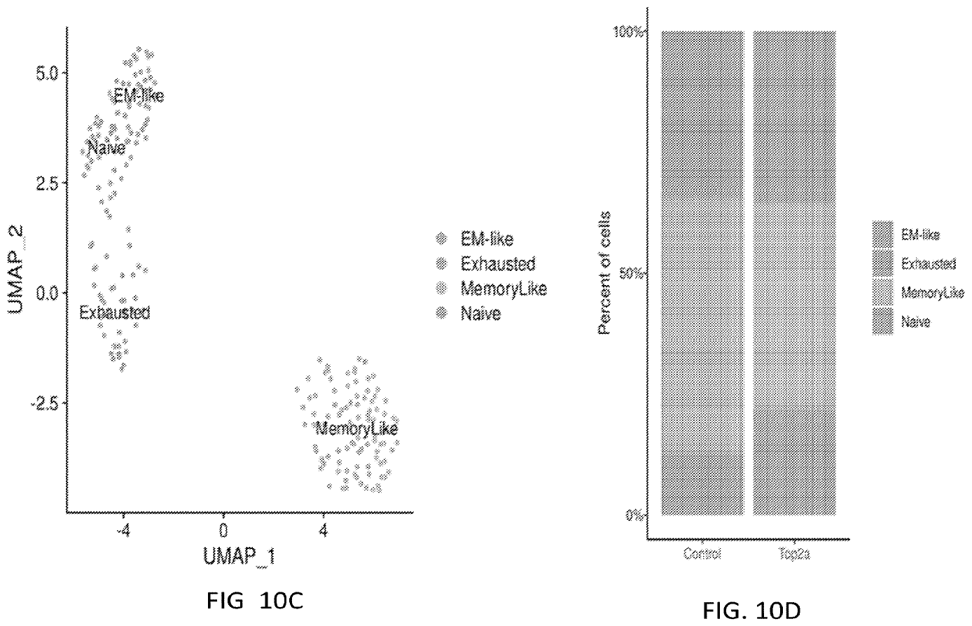

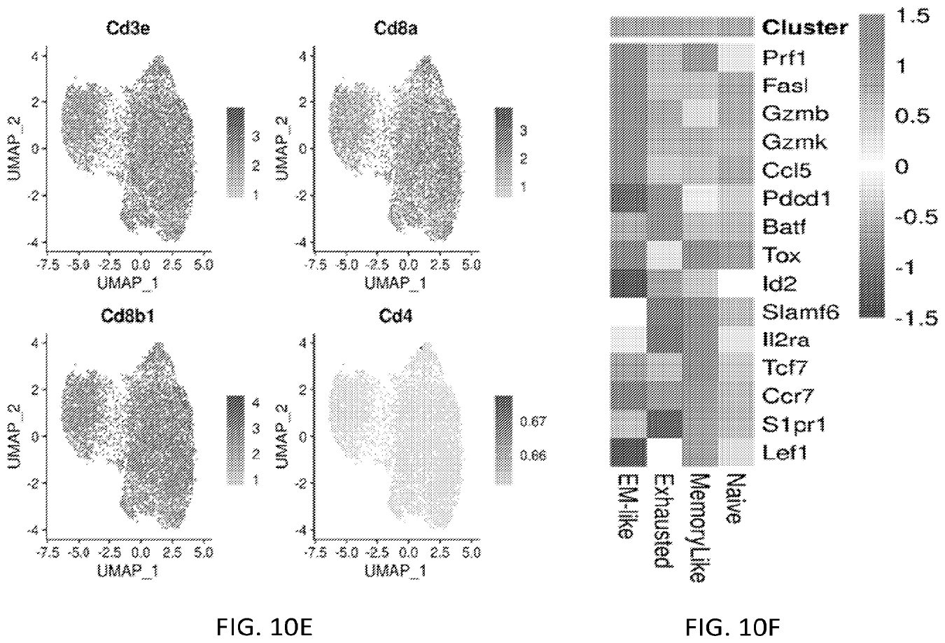

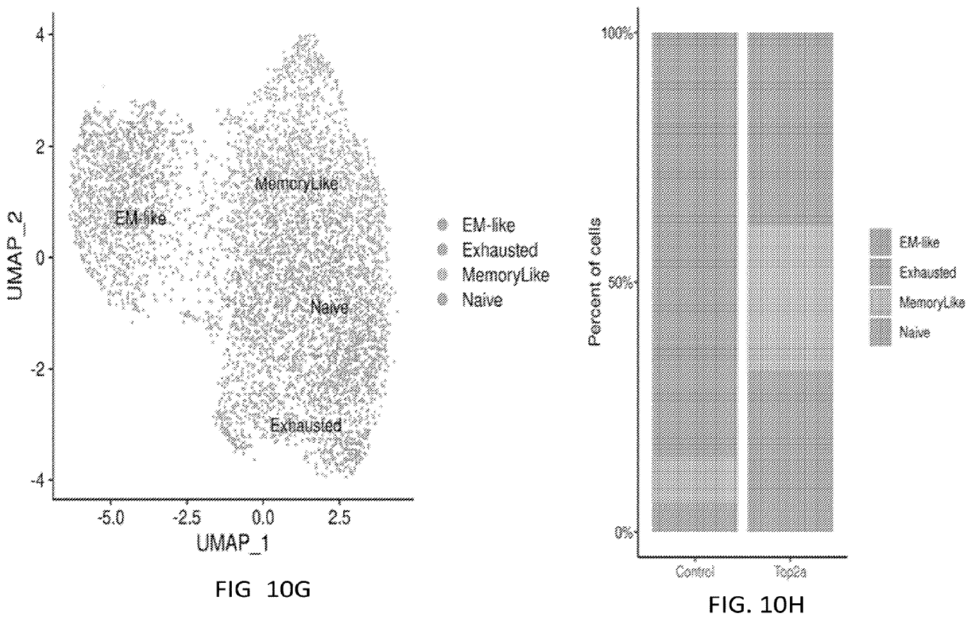

To determine the effects of Top2A vaccine treatment on different T cell subsets, deep clustering of CD8+ T cells, CD4+ T cells and DNT cells from mouse breast tumors was performed. Location of the CD8+ T cells was identified by the canonical markers (FIG. 10A). Unsupervised clustering of CD8+ T cells using the TILPRED program (https://github.com/carmonalab/TILPRED) (Carmona et al., 2020) identified four CD8+ T cell subsets with distinct transcriptomic profiles (FIG. 10B,C). The CD8 subsets included effector-memory (EM)-like, exhausted, memory-like, and naïve CD8+ T cells. In cancer, the EM-like and exhausted CD8+ T cells are involved in anti-tumor and pro-tumor functions, respectively. Top2A vaccination greatly increased abundance of the anti-tumor EM-like CD8+ T cells in the breast tumor microenvironment (TME) (FIG. 10D). In contrast, the exhausted CD8+ T cells were decreased by the Top2A vaccine treatment. These data suggest that the Top2A vaccine treatment improves overall composition of beneficial anti-tumor CD8+ T cells in breast tumors. In the lymph node samples from tumor-bearing mice, we observed similar effects of Top2A vaccine treatment on CD8+ T cell subsets. CD8+ T cells from the lymph node samples were plotted (FIG. 10E), and CD8+ T cells subsets identified using corresponding markers (FIG. 10F,G). Top2A vaccine treatment increased the abundance of EM-like CD8+ T cells and greatly reduced the proportion of exhausted CD8+ T cells in the lymph nodes (FIG. 10H).

### Example 10—Top2A Vaccine Treatment Increased the Abundance of CD4+Th1 Cells in Mouse Breast Tumor and Lymph Node Tissues

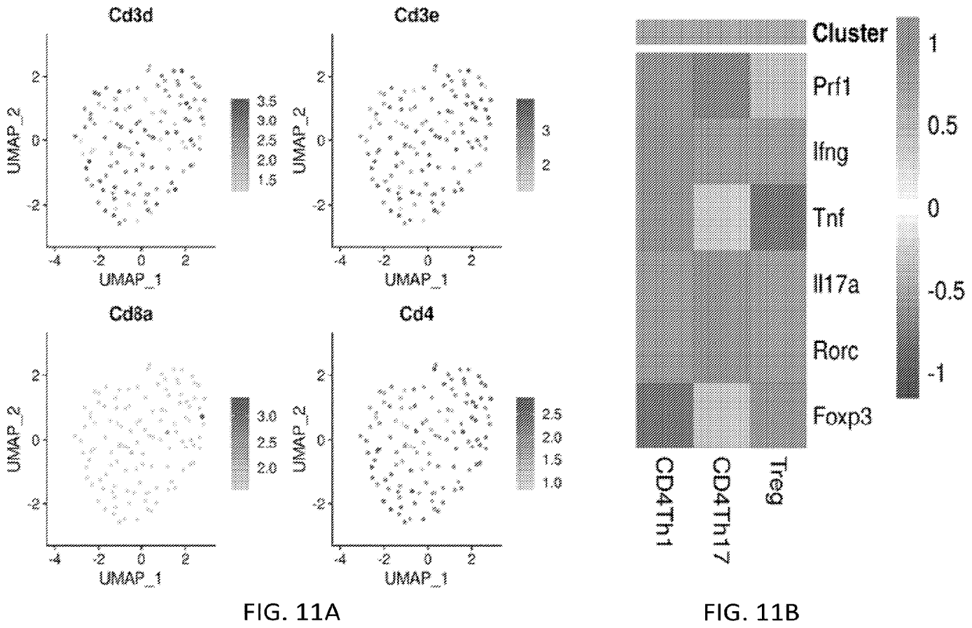

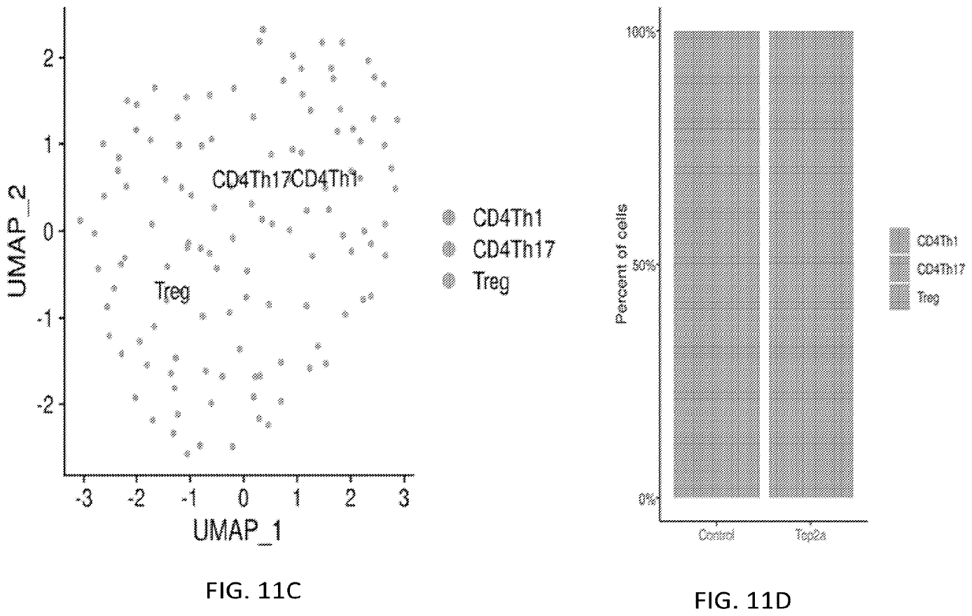

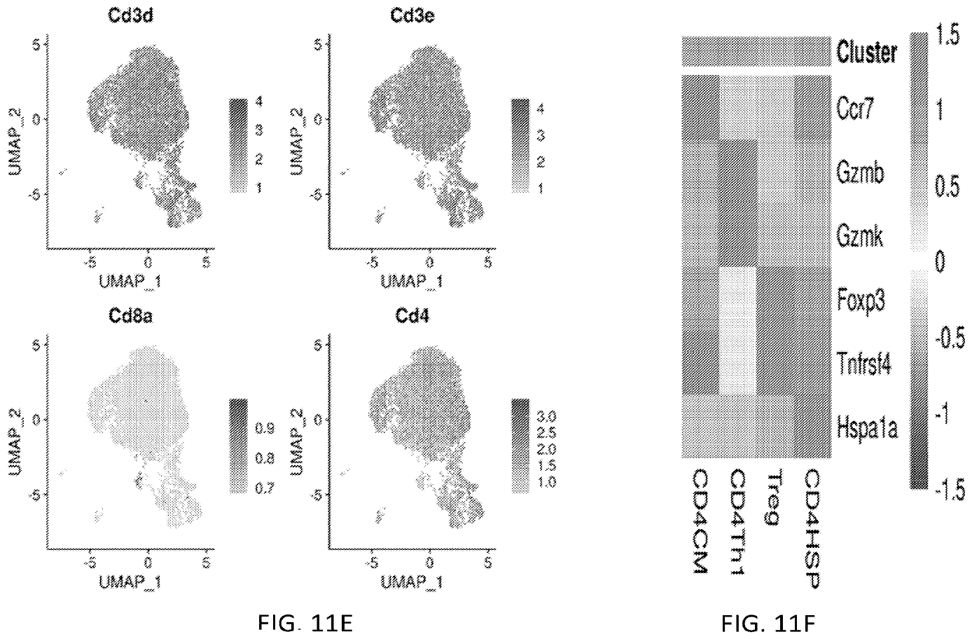

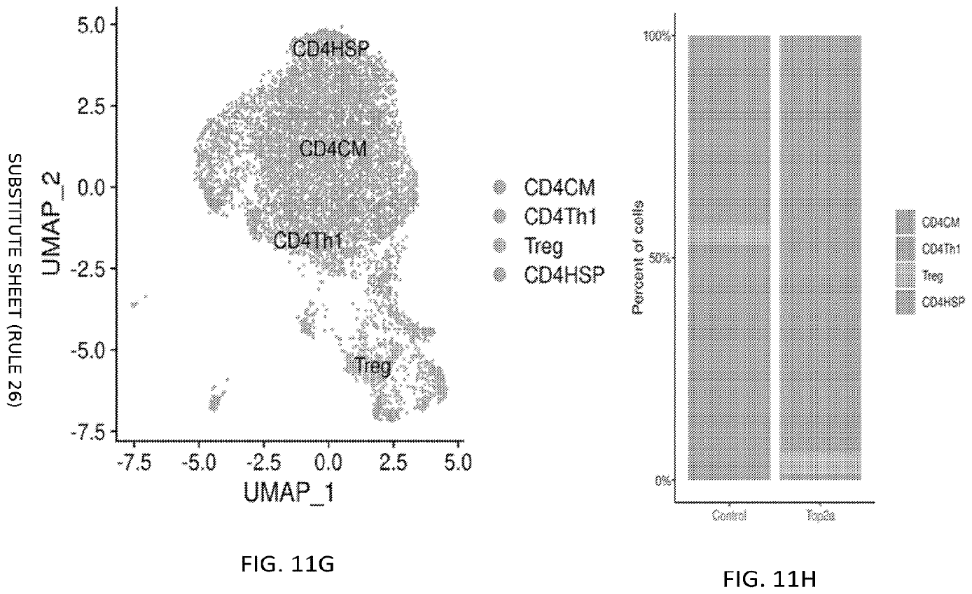

Functional CD4+ cells could play a major role in the anti-tumor response induced by Top2A vaccine treatment. CD4+ T cells from the breast tumors were analyzed and three types of CD4+ T cell subsets were identified including CD4 Th1, CD4 Th17 and Treg cells (FIG. 11A-C). Top2A vaccine treatment significantly increased the proportion of CD4+Th1 and Th17 cells in breast tumors while decreasing the abundance of Treg cells (FIG. 11D). CD4+Th1 cells overexpressed the marker genes perforin, IFNg, and TNF; CD4 Th17 T cells overexpressed IL17a and Rorc; Treg cells overexpressed Foxp3 (FIG. 11B). These results suggest that the anti-tumor function of CD4+ T cells was significantly enhanced by the Top2A vaccine treatment. In the lymph node samples, four types of CD4+ T cells subsets were identified: CD4 CM (central-memory T cells), CD4 Th1, Treg and CD4HSP (CD4+ T cells overexpressing Hspa1a) (FIG. 11E-G). Similar to the breast tumor samples, we observed increased frequencies of CD4+Th1 cells after Top2A vaccination (FIG. 11H). CD4 CM cells were also increased, while the immune inhibitory CD4 HSP cells were decreased by the Top2A vaccine treatment (FIG. 11H). Our results suggest that CD4+Th1 cells are involved in the anti-tumor response generated by the Top2A vaccine.

### Example 11—Top2A Vaccine Treatment Also Impacts Other Immune Cells in Mouse Tumor and Lymph Node Tissues

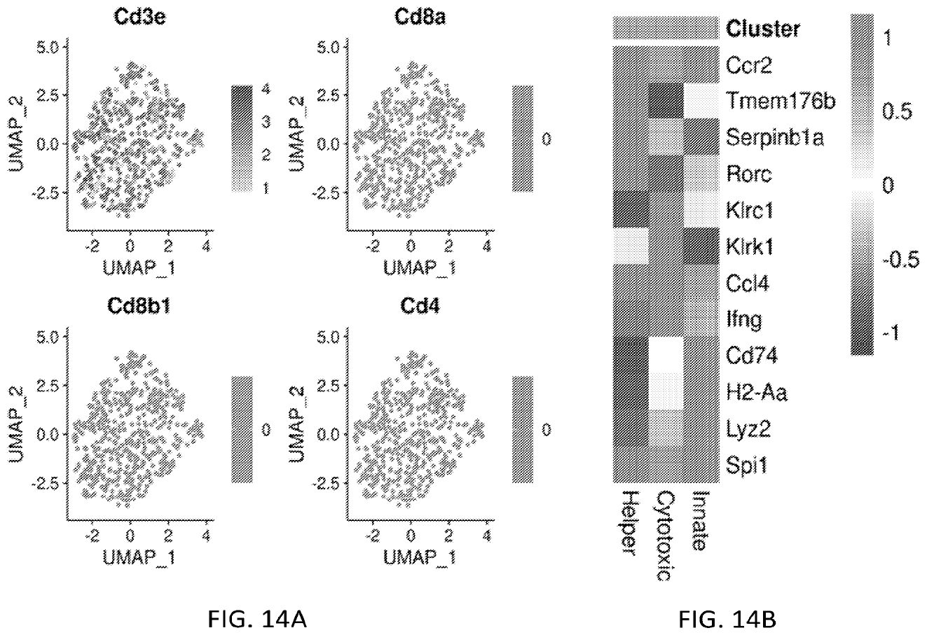

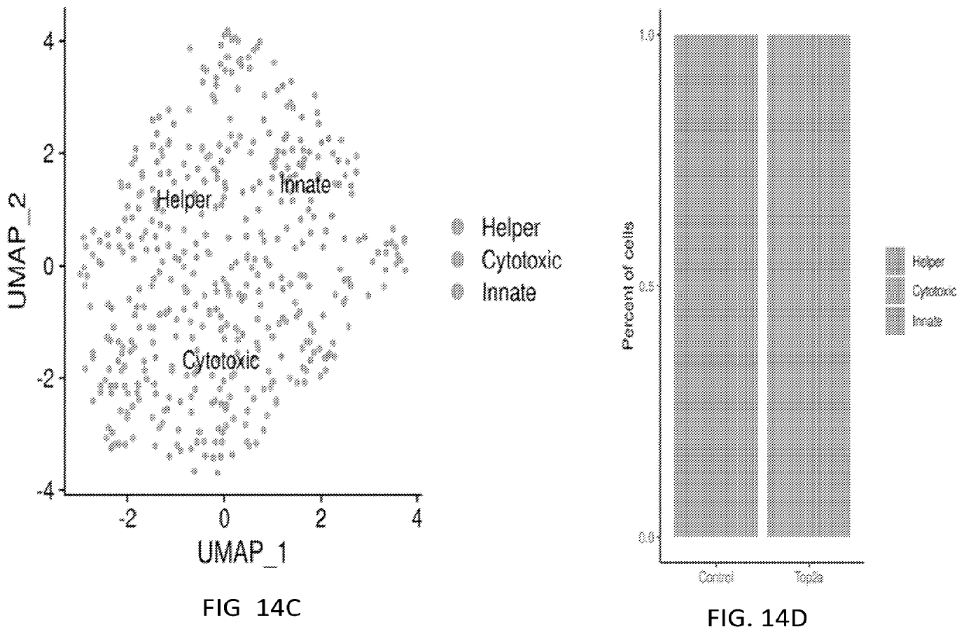

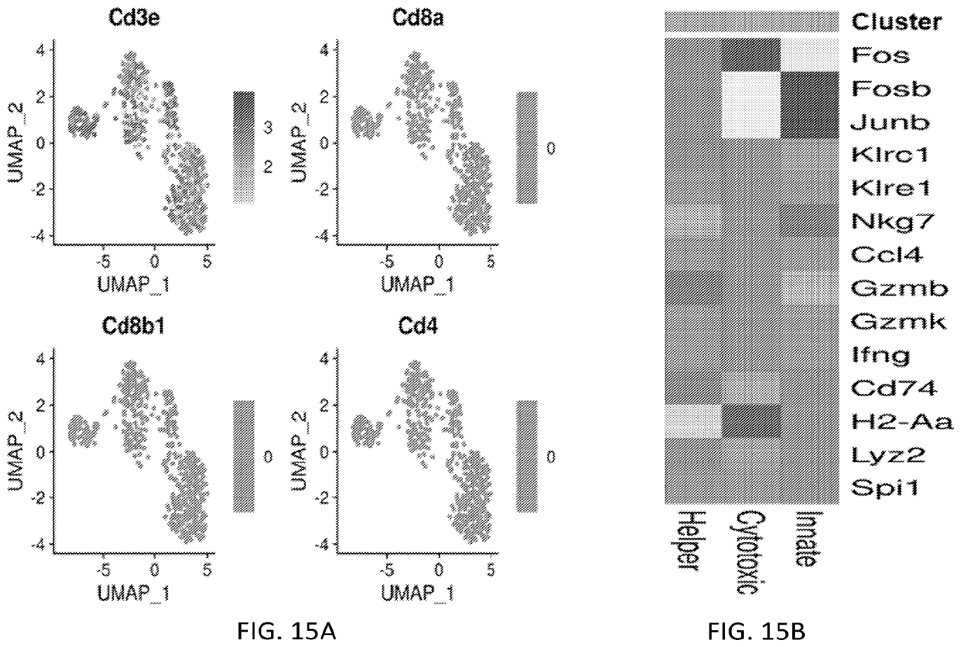

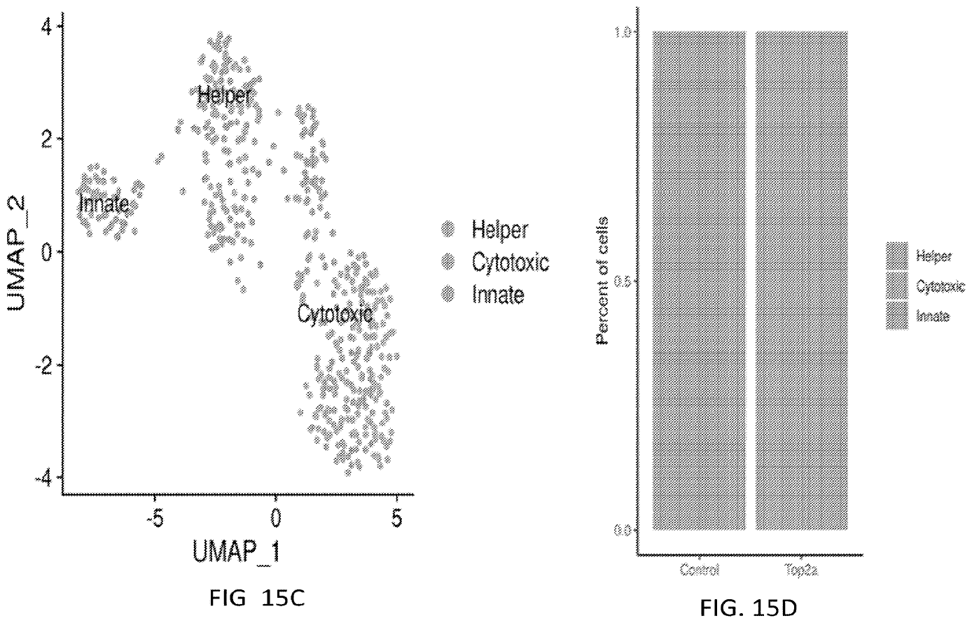

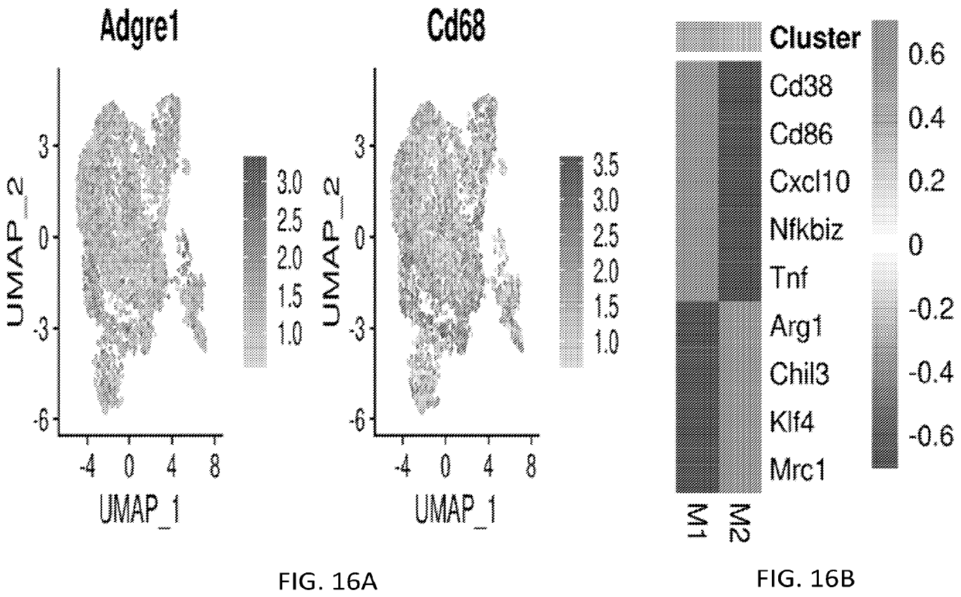

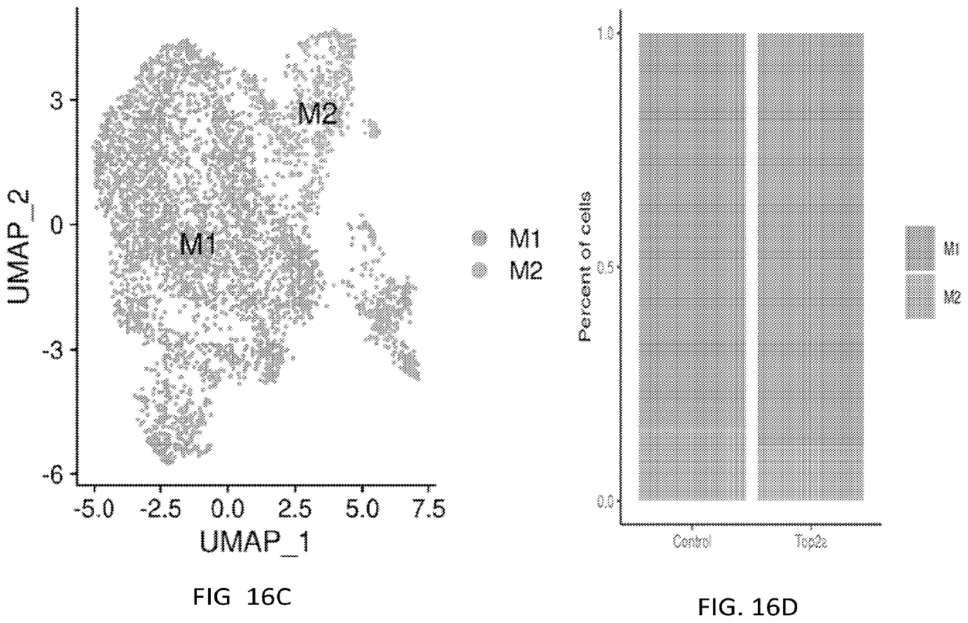

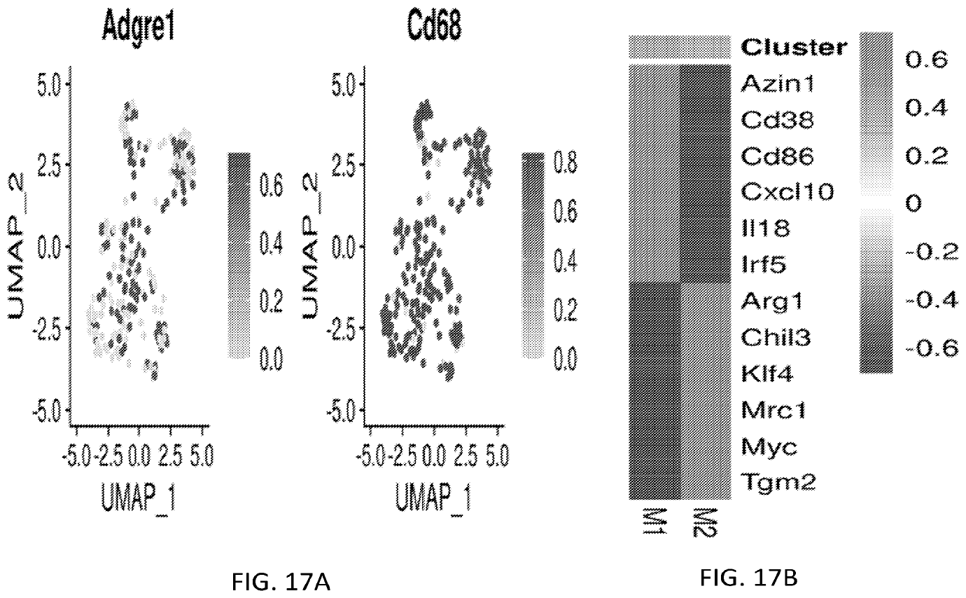

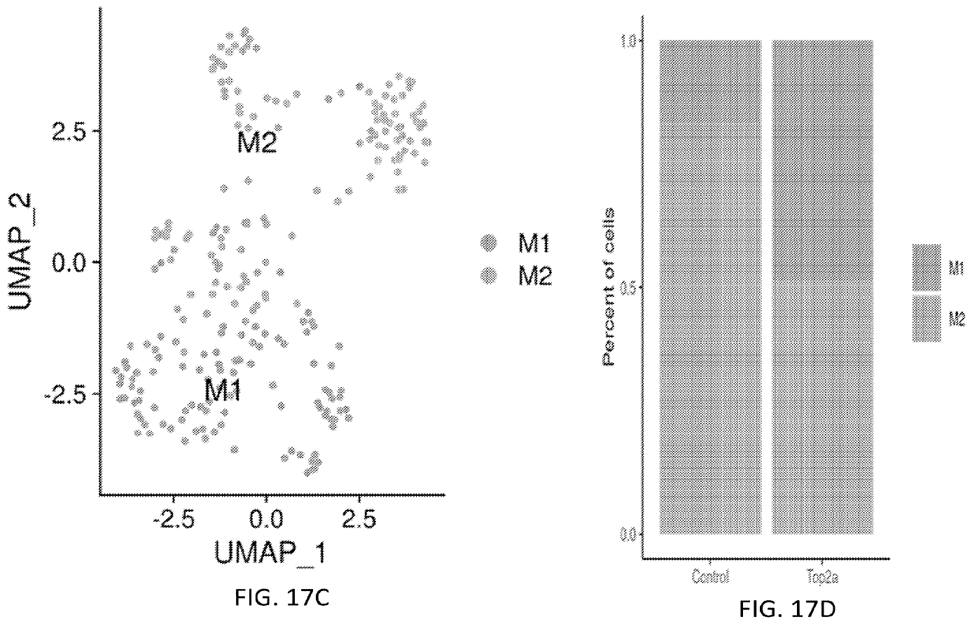

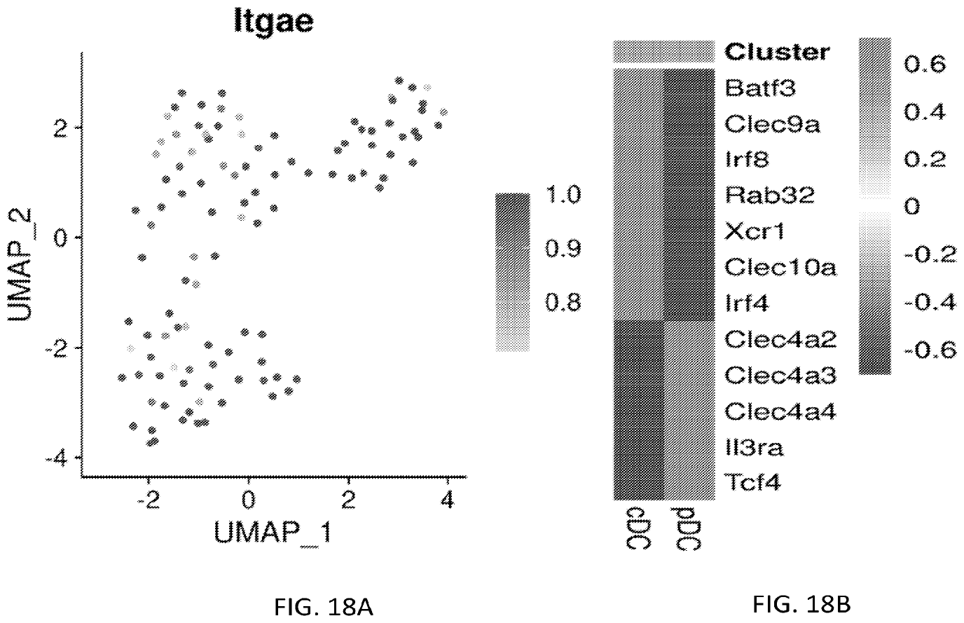

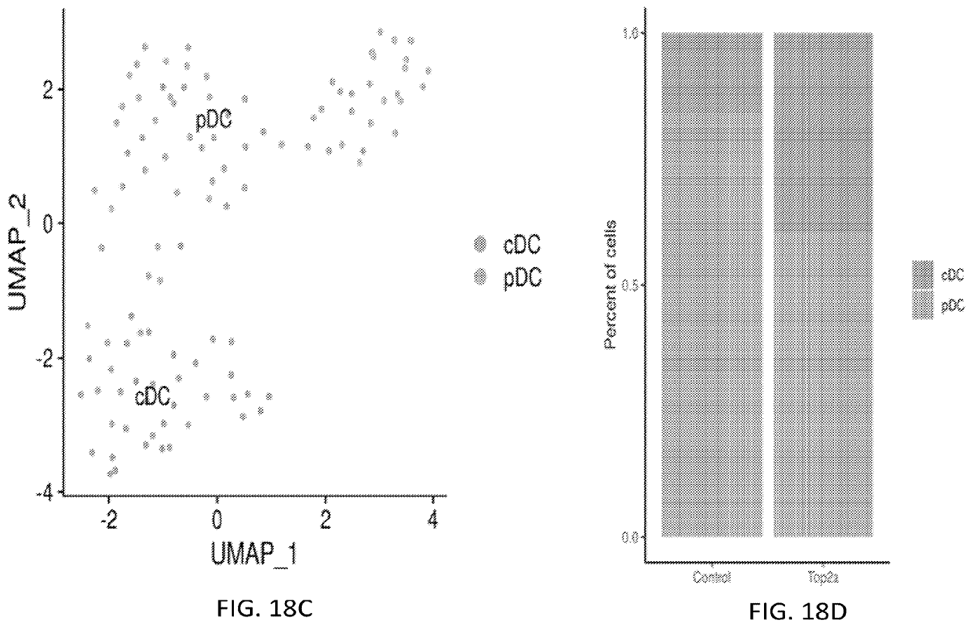

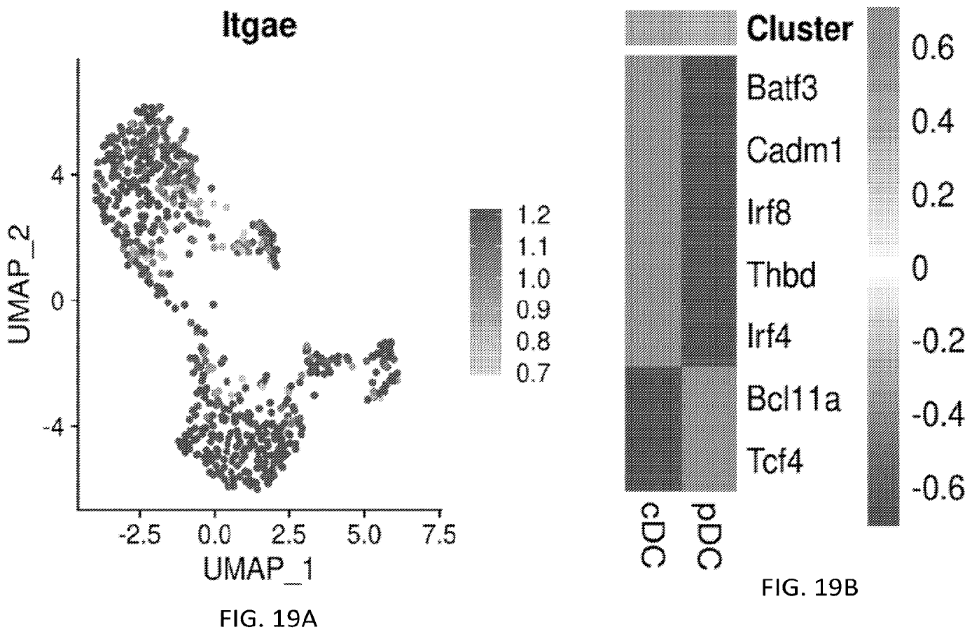

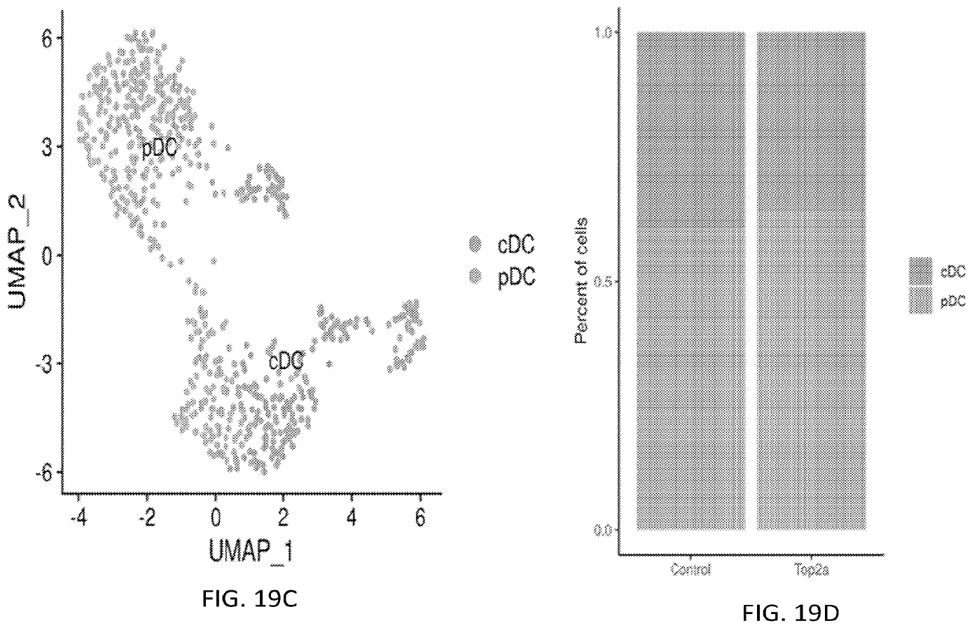

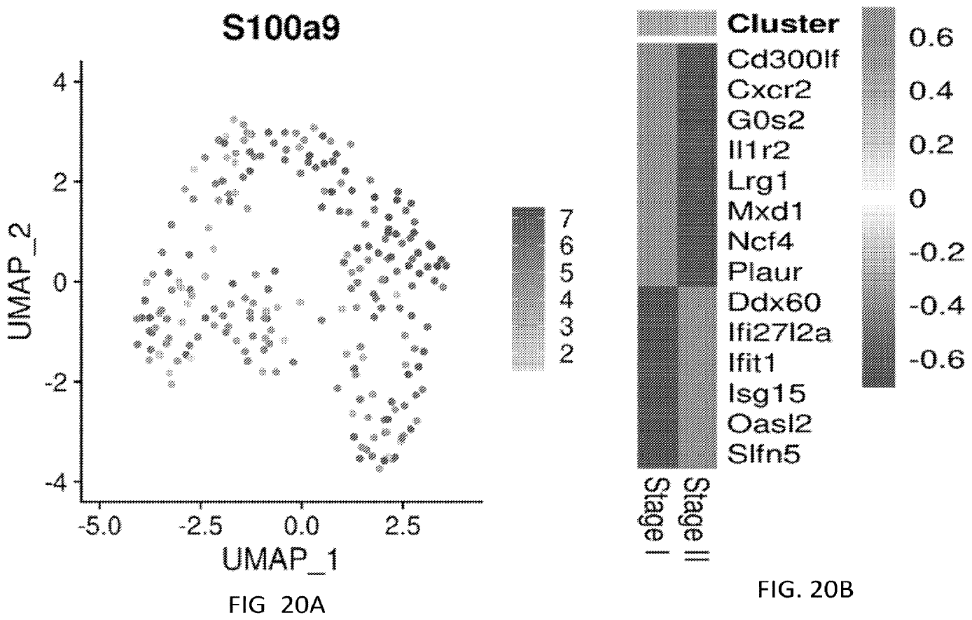

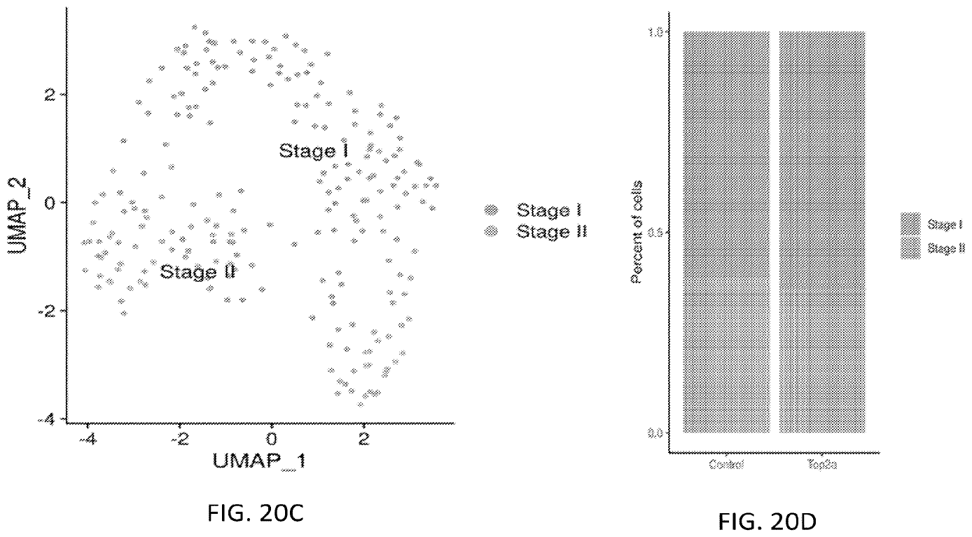

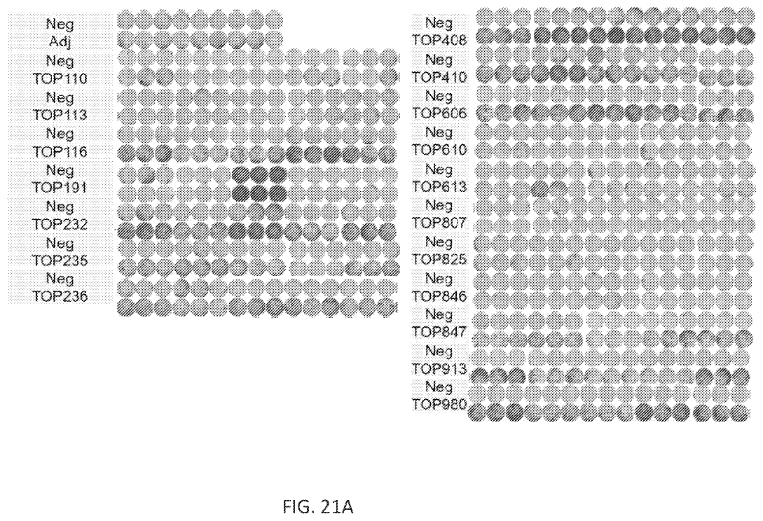

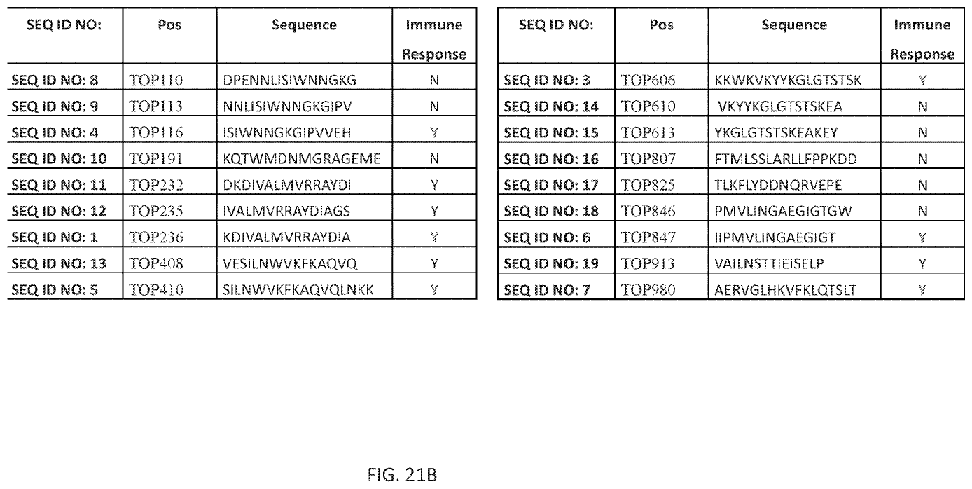

Tumor and lymph node tissues were examined for changes in other immune cell populations after Top2A vaccine treatment. For the DNT cells (CD4/CD8 double-negative T cells) in mouse breast tumor and lymph node tissues, the cells were divided into three subsets: helper, cytotoxic, and innate DNT cells (FIGS. 14 and 15) (Yang et al., 2021). Top2A vaccine treatment significantly increased the abundance of cytotoxic DNT cells, while the treatment decreased the proportion of innate DNT cells in mouse breast tumor tissues (FIG. 14D). The percentage of helper DNT cells was not altered by Top2A vaccine vaccination (Supplemental FIG. 14D). For the lymph node samples, Top2A vaccine treatment increased the percentage of helper DNT cells but did not change the percentage of cytotoxic DNT cells (Supplemental FIG. 15D). These results suggest that the Top2A vaccine treatment specifically increases abundance of cytotoxic DNT cells in mouse breast tumors. Macrophages were subdivided into the M1 and M2 macrophages according to their marker gene expression in both breast tumor and lymph node tissues (FIGS. 16 and 17). Abundance of anti-tumor M1 macrophages was increased and the pro-tumor M2 macrophages decreased after Top2A vaccination in both breast tumor and lymph node tissues (FIGS. 16D and 17D). These data suggest that macrophages may also participate in the anti-tumor response induced by Top2A vaccination. Two subsets of DC were detected in tissues of treated mice: conventional dendritic cells (CDC) and plasmacytoid dendritic cells (pDC) based on the analysis of corresponding marker genes (FIGS. 18 and 19) (Villar et al., 2020). The Top2A vaccine treatment significantly increased the proportion of cDCs in breast tumor tissues but not in lymph nodes. cDC have been known to play a critical role in anti-tumor immunity (Murphy et al. 2022). Finally, neutrophils were detected in the breast tumor samples. Neutrophils can be divided into Stage I and Stage II subsets (Giladi et al., 2022), which represent progenitor and mature neutrophils, respectively. Both neutrophil subsets in breast tumor tissues were identified, and found that the Top2A vaccine treatment did not change the proportions of these two subsets (FIGS. 20A-20C).

### Example 12—TCR Clonotype Analysis Revealed Appearance of New T Cell Clones as a Consequence of Top2A Vaccination

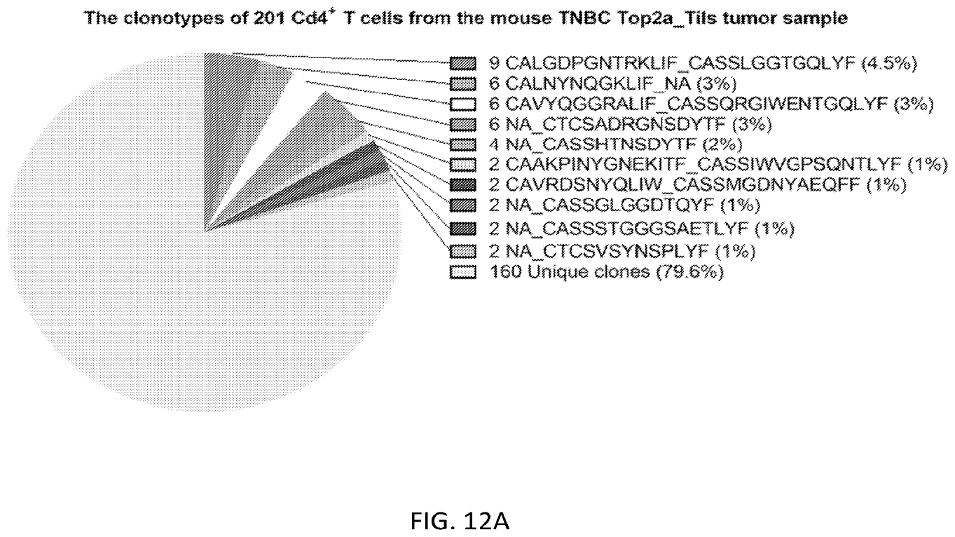

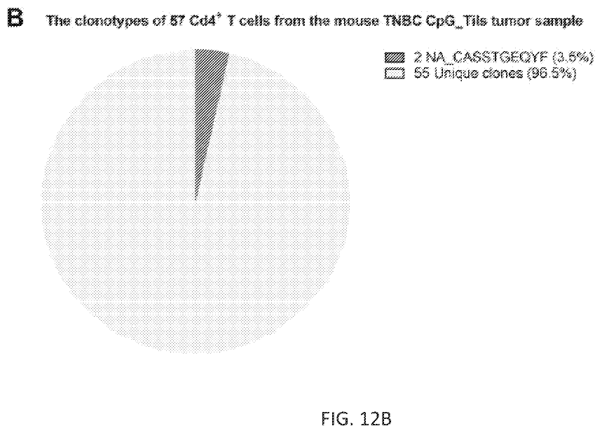

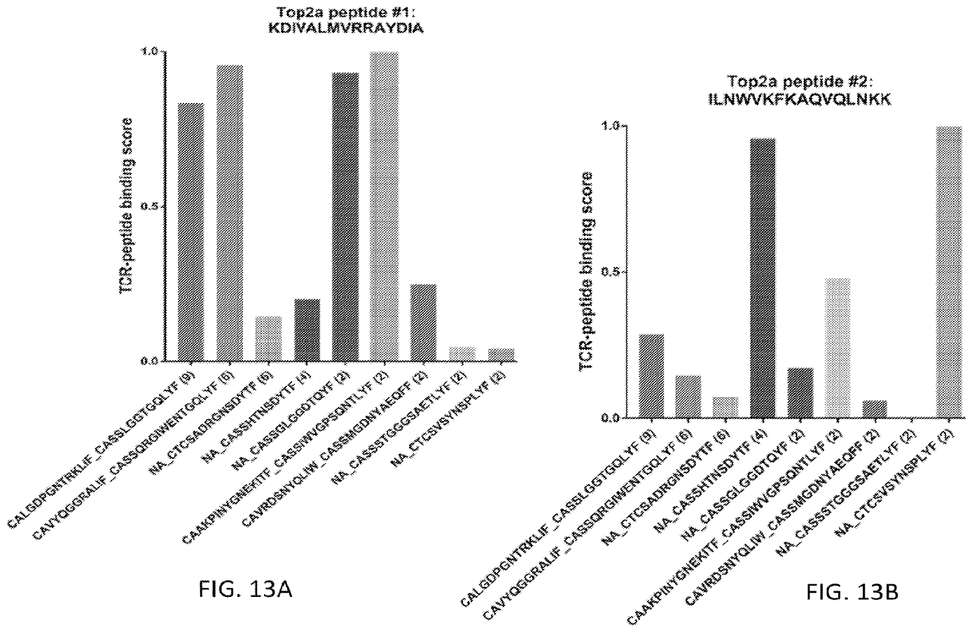

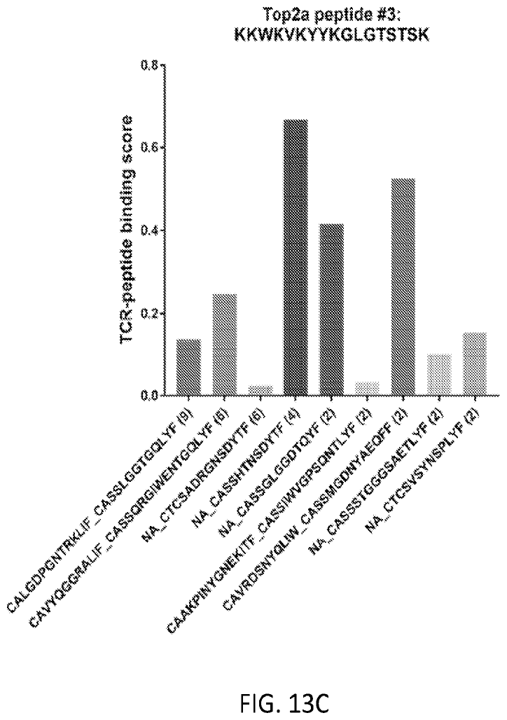

To explore potential mechanisms underlying the anti-tumor response induced by Top2A vaccine treatment, scTCRseq data of T cells from the control and Top2A vaccine groups was analyzed. 201 CD4+ T cells in tumor tissues from Top2A vaccinated mice and 57 CD4+ T cells in tumor tissues from the CpG control mice were sequenced. The top five most frequent TCR clonotypes (frequency >=4) constituted 15.5% of the total CD4+ TCR clonotypes in Top2A vaccine group; these clonotypes were not found in CpG control group, suggesting their involvement in the Top2A vaccine-generated CD4+ T cell immune response (FIG. 12A). Nearly all of the TCR clonotypes in vaccinated mice of were unique versus the CpG controls (FIG. 12). To predict likelihood of TCR-peptide binding for the Top2A vaccine peptides, ERGO (pEptide tcR matchinG prediction) software was used which is a highly specific and generic TCR-peptide binding predictor (Springer et al., 2020) (https://github.com/louzounlab/ERGO). For the first Top2A peptide-KDIVALMURRAYDIA (SEQ ID NO: 1), the top 4 TCR clonotypes with the highest binding scores include: CAAKPINYGNEKITF_CASSIWVGPSQNTLYF (2 cells, clonotype frequency: 1%), CAVYQGGRALIF_CASSQRGIWENTGQLYF (6 cells, clonotype frequency: 3%), NA_CASSGLGGDTQYF (2 cells, clonotype frequency: 1%), and CALGDPGNTRKLIF_CASSLGGTGQLYF (9 cells, clonotype frequency: 4.5%) (FIG. 13A). For the second Top2A peptide-ILNWVKFKAQVQLNKK (SEQ ID NO: 2), the top 4 clonotypes with the highest binding scores were: NA_CTCSVSYNSPLYF (2 cells, clonotype frequency: 1%), NA_CASSHTNSDYTF (4 cells, clonotype frequency: 2%), CAAKPINYGNEKITF_CASSIWVGPSQNTLYF (2 cells, clonotype frequency: 1%), and CALGDPGNTRKLIF_CASSLGGTGQLYF (9 cells, clonotype frequency: 4.5%) (FIG. 13B). For the third Top2A peptide-KKWKVKYYKGLGTSTSK (SEQ ID NO: 3), the top 4 clonotypes with the highest binding scores are: NA_CASSHTNSDYTF (4 cells, clonotype frequency: 2%), CAVRDSNYQLIW_CASSMGDNYAEQFF (2 cells, clonotype frequency: 1%), NA_CASSGLGGDTQYF (2 cells, clonotype frequency: 1%), and CAVYQGGRALIF_CASSQRGIWENTGQLYF (6 cells, clonotype frequency: 3%) (FIG. 13C). Notably, the top two most frequent TCR clonotypes, CALGDPGNTRKLIF_CASSLGGTGQLYF and CAVYQGGRALIF_CASSQRGIWENTGQLYF, both showed high binding affinity to the Top2A peptide KDIVALMVRRAYDIA (SEQ ID NO: 1) (FIG. 13A), suggesting that this Top2A peptide plays an important role in the Top2a vaccine-induced anti-tumor T cell response.

### Discussion

Top2A is an enzyme that controls the topological states of DNA. It catalyzes double-stranded DNA breaks and promotes gene transcription during mitosis (Pei, et al., 2018), and has been implicated in several malignancies including breast cancer, ovarian cancer and prostate cancer. Top2A is directly associated with tumor cell proliferation and invasiveness in breast cancer (Klintman et al., 2016), and expression has been reported to be amplified in TNBC patients with high risk factors such as large tumor size, high-grade tumor, and lymph node infiltration (Zheng et al., 2016; Nakagawa et al., 2011).

Here, overexpression of the Top2A gene in both mouse breast tumor cell lines and human TNBC cancer samples (FIG. 1) was identified. The expression of Top2A in TNBCs from AA women was confirmed. Recently, the incidence of TNBC has been reported to be higher in AA women (Carey et al., 2006) and is associated with a poorer prognosis as compared to European American women (Carey et al., 2006), presumably due to various sociologic factors in addition to the increased likelihood of developing TNBC (Siddharth et al., 2018). Top2a protein expression levels in human TNBC tissue and mouse TNBC tumors were evaluated, and confirmed overexpression at the protein level (FIG. 2).

CD4 T cells can differentiate into various Th subsets, which can induce, modulate and maintain immune responses to tumor antigens. These CD4 T cell subsets include Th1, Th2, and regulatory T cells (Tregs). The Th1 subset produces IFN-γ, TNF-α and IL-2, which regulate cellular immunity and play important roles in anti-tumor immune responses (Pan et al, 2016). The Top2a vaccine employed in the current study induced predominantly Th1 cell and APC responses, without eliciting a strong Th2 response (FIG. 7). Type I cytokines secreted by Th1 cells, such as IFN-γ, can up-regulate MHC class I expression on the membrane of tumor cells, as well as APC, which can facilitate tumor recognition by CD8+ T cells (Zhou, 2009). Moreover, Th1 cells could also facilitate cross-presentation of MHC class I binding peptide antigens to CD8+ T cells (Matsuo et al., 2004; Disis et al., 2013). These mechanisms could be involved in generating the Th1 responses associated with the Top2A vaccine, and ultimately facilitating generation of a CD8+ T cell response. Cytotoxic CD8+ T cells play an important role in the anti-tumor immune response by direct killing of tumor cells (Martinez-Lostao et al., 2015). Our study demonstrated that the Top2A vaccine could stimulate both local and systemic antigen-specific CD4+ and CD8+ T cell responses. A significant increase in IL-23 secretion from splenocytes isolated from Top2A vaccinated mice was found. This is notable as IL-23 plays is known to play a role in antitumor activity (Lo et al., 2003; Ma et al., 2020). in both syngeneic and GEM models. Meanwhile, no toxicity was observed in vaccinated mice. 15-17 amino acid peptides were designed with 100% sequence identity between human and mouse Top2a, making it possible that these peptides could be translated into clinical studies. Overall, the data herein demonstrate that the multi-peptide Top2A vaccine is highly immunogenic and has the potential to be efficacious in the immunoprevention of TNBC.

### Example 13—Additional Screen for Top2A Peptides

In this Example, additional peptide candidates were identified using a multi-scoring system that combines multiple MHC class II peptide binding algorithms, as was done above in Example 3. The additional 18 peptide candidates are listed below in Table 2.

TABLE 2
Top2A Peptides - Additional Screen

| SEQ ID NO: | Sequence | Immune Response |
|---|---|---|
| SEQ ID NO: 8 | DPENNLISIWNNGKG | N |
| SEQ ID NO: 9 | NNLISIWNNGKGIPV | N |
| SEQ ID NO: 4 | ISIWNNGKGIPVVEH | Y |
| SEQ ID NO: 10 | KQTWMDNMGRAGEME | N |
| SEQ ID NO: 11 | DKDIVALMVRRAYDI | Y |
| SEQ ID NO: 12 | IVALMVRRAYDIAGS | Y |
| SEQ ID NO: 1 | KDIVALMVRRAYDIA | Y |
| SEQ ID NO: 13 | VESILNWVKFKAQVQ | Y |
| SEQ ID NO: 5 | SILNWVKFKAQVQLNKK | Y |
| SEQ ID NO: 3 | KKWKVKYYKGLGTSTSK | Y |
| SEQ ID NO: 14 | VKYYKGLGTSTSKEA | N |
| SEQ ID NO: 15 | YKGLGTSTSKEAKEY | N |
| SEQ ID NO: 16 | FTMLSSLARLLFPPKDD | N |
| SEQ ID NO: 17 | TLKFLYDDNQRVEPE | N |
| SEQ ID NO: 18 | PMVLINGAEGIGTGW | N |
| SEQ ID NO: 6 | IIPMVLINGAEGIGT | Y |
| SEQ ID NO: 19 | VAILNSTTIEISELP | Y |
| SEQ ID NO: 7 | AERVGLHKVFKLQTSLT | Y |

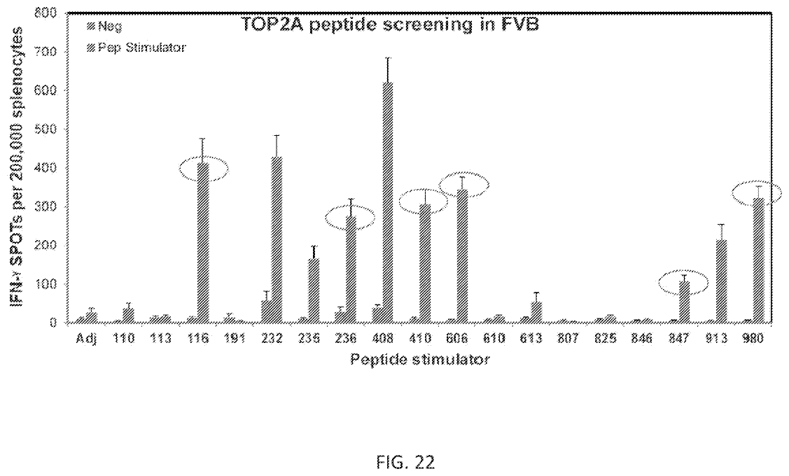

The immunogenicity of the Top2A peptides of Table 2 were evaluated in C3 (1)/Tag-REAR mice via IFN-γ ELISPOT assays with a negative control of HIV peptides, as described above with reference to Example 3. The IFN-γ ELISPOT plate is depicted in FIG. 21 and the quantified results are provided in FIG. 22. As can be seen in FIG. 22 several of the peptides did not exhibit significant immunogenic potential. Some peptides in this Example did exhibit immunogenic potential and also exhibit sufficient solubility to be selected as potential Top2A peptide candidates for the compositions disclosed herein—these include peptides associated with the circled data on the bar graph of FIG. 22 (SEQ ID NOS: 4, 5, 6, 7, 8, and 9).

### Example 14—Top2A Peptide Vaccination Inhibited Lung Tumor Progression in Syngeneic Models of Lung Cancer

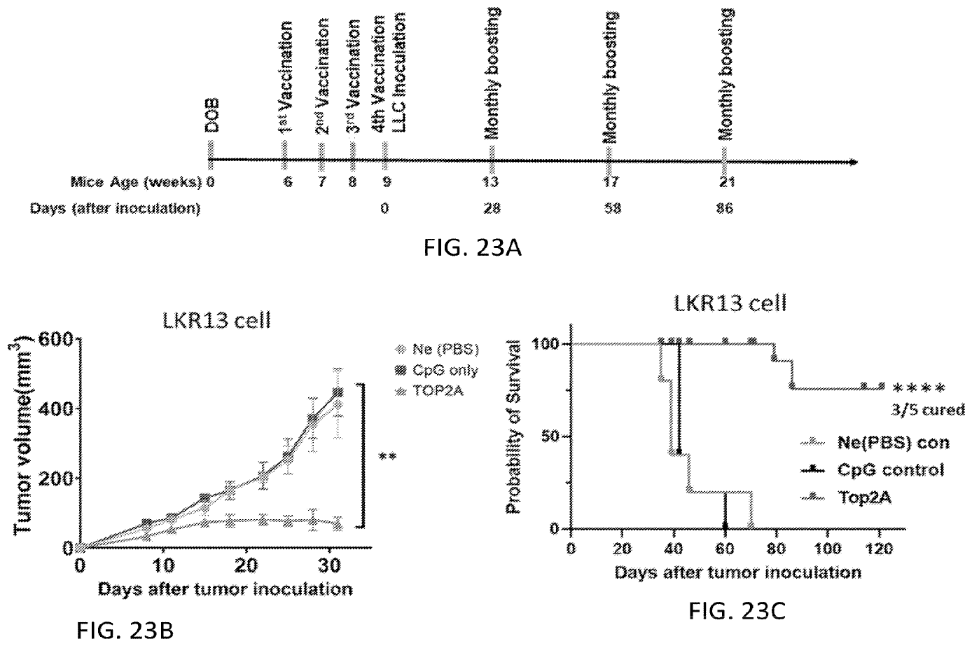

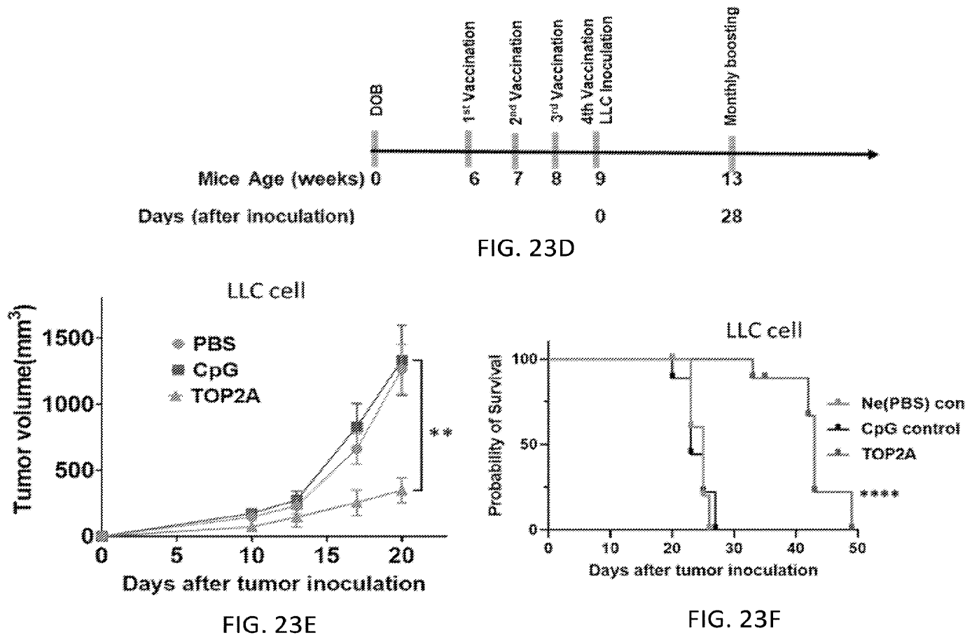

In addition to breast cancer, TOP2A is highly overexpressed/amplified in many tumor types including lung cancer. We therefore hypothesized that TOP2A vaccination will exhibit similar immunopreventive efficacy against lung cancer. Using a similar approach, efficacy of the TOP2A vaccine was tested in two syngeneic lung cancer mouse models (using LKR13 and LLC lung adenocarcinoma cell lines). Significant preventive effects were found in mice vaccinated with the TOP2A vaccine (FIGS. 23A-23F). In LKR13 cell model, the mean tumor volumes on day 31 was 446.3 mm³ in CpG control vs. 69.6 mm³ in Top2A vaccine (FIG. 23B, p<0.01). In LLC cell model, the mean tumor volumes on day 20 was 1330.2 mm³ in CpG control vs. 348.4 mm³ in Top2A vaccine (FIG. 23E, p<0.01). Furthermore, survival of animals treated with the TOP2A vaccine was significantly prolonged compared with adjuvant treated mice in both mouse LUAD models (FIGS. 23C&F, p<0.001, log-rank test). Based on the unexpected results in this example, the Top2A compositions disclosed herein are a promising candidate for immunoprevention of lung cancer.

### Example 15—Design and Testing of MHC II Top2A mRNA Vaccine

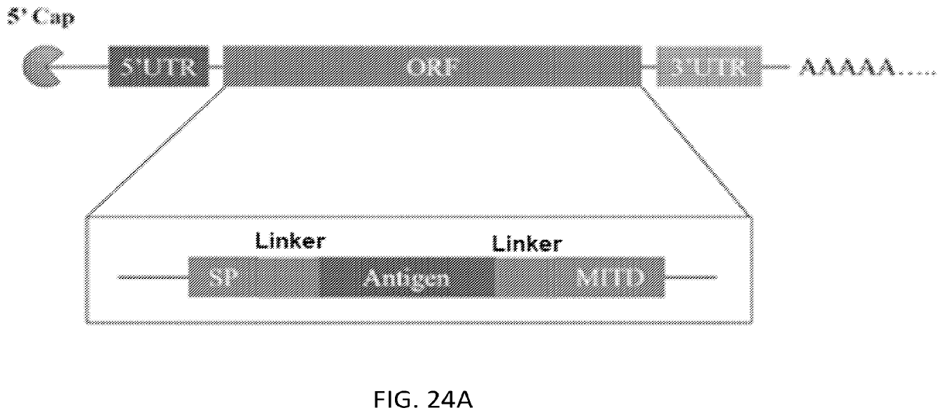

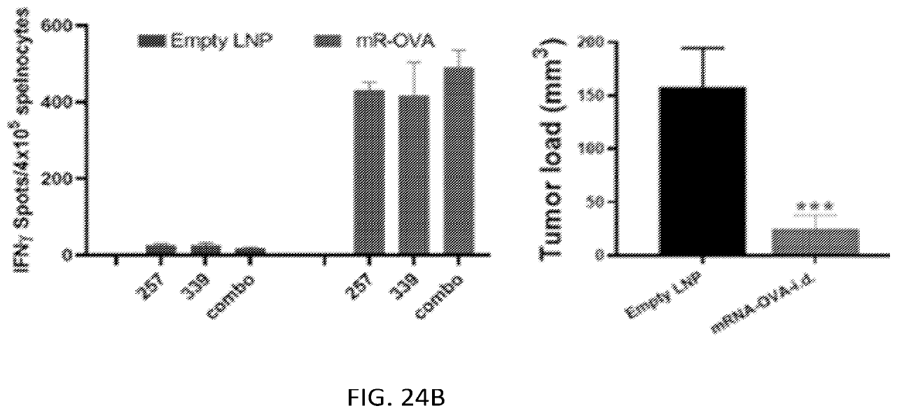

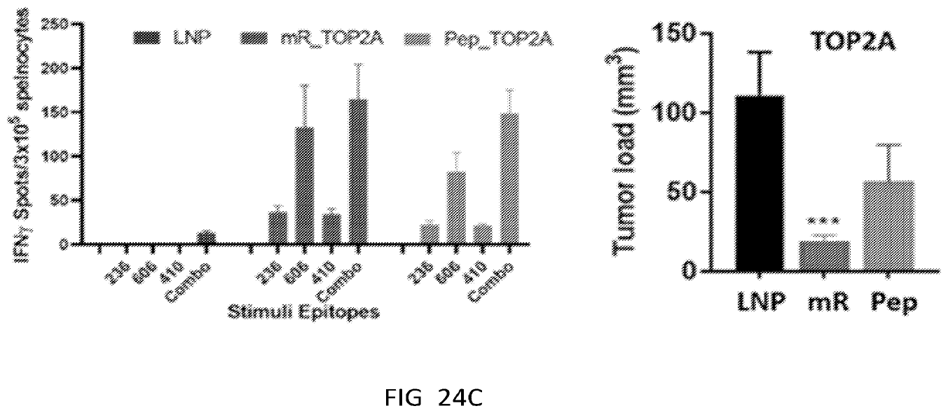

Both OVA-mRNA and MHC II TOP2A-mRNA LNPs were generated using Moderna's LNP (M-LNP) (FIGS. 24A-24C) and evaluated in B16 (OVA) or LKR13 (TOP2A) lung metastasis models. mRNA vaccines containing MHC-II epitopes specific to TOP2A were tested in a syngeneic animal model, also compared to peptide vaccination. Both OVA (positive control) (FIG. 24B) and TOP2A (FIG. 24C) mRNA vaccines (3 weekly doses of 10 μg/mouse s.c.) generated strong antitumor immunity and significantly reduced metastatic burden in the lung metastasis syngeneic models. Significantly stronger antitumor efficacy (>90%) after vaccinations by TOP2A-mRNA-LNP compared to the peptide/adjuvant TOP2A vaccine was demonstrated (FIG. 24C). In the case of TOP2A-mRNA-LNP, three epitopes derived MHC II epitopes separated by linkers were included. The mRNA encodes for the amino acid sequence provided below (SEQ ID NO: 31). The lipid nanoparticle (LNP) used was an FDA-approved LNP from Moderna called SM-102, which is a synthetic amino lipid utilized alongside other lipids to compose lipid nanoparticles. Previously, SM-102 is integral to the drug delivery system of the Moderna COVID-19 vaccine. SM-102 is an ionizable lipid that maintains a near-neutral charge at physiological pH but becomes positively charged within the nanoparticle structure (where the amine group is protonated to form an ammonium cation). This enables them to bind effectively to the negatively charged mRNA backbone. The remainder of the nanoparticle consists of PEGylated lipids, which enhance particle stability, along with phospholipids and cholesterol molecules contributing to the particle's structure.

TOP2A-mRNA peptide sequence
(SEQ ID NO: 31)
MRVTAPRTLILLLSGALALTETWAGGSGGGGSGGGIVESILNWVKFKAQV
QLNKKCSAVKGGSGGGGSGGMQSLDKDIVALMVRRAYDIAGSTKDGGSGG
GGSGGSTPNHKKWKVKYYKGLGTSTSKEAKEYGGSGGGGSGGIVGIVAGL
AVLAVVVIGAVVATVMCRRKSSGGKGGSYSQAASSDSAQGSDVSLTA

### Example 16—Identification of MHC I Top2A Epitopes

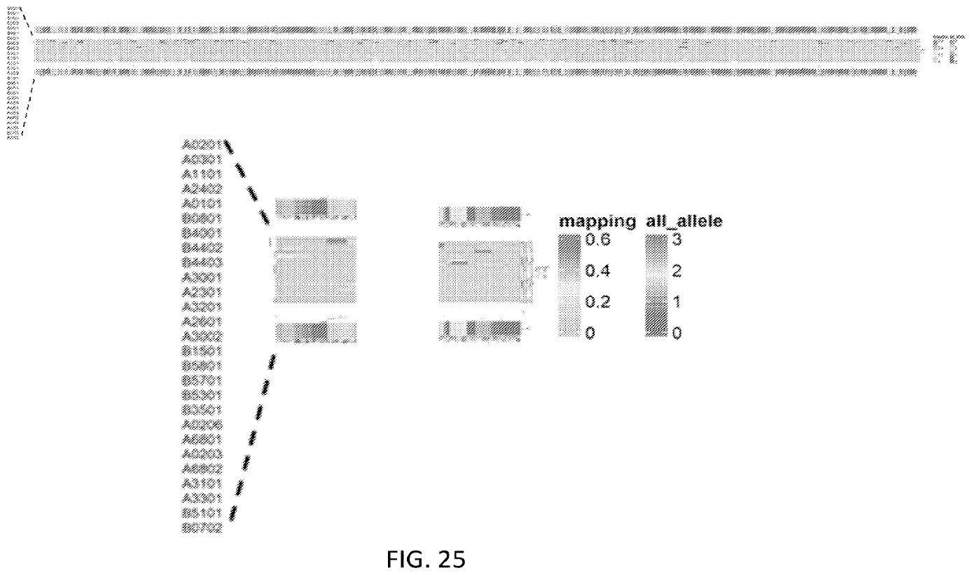

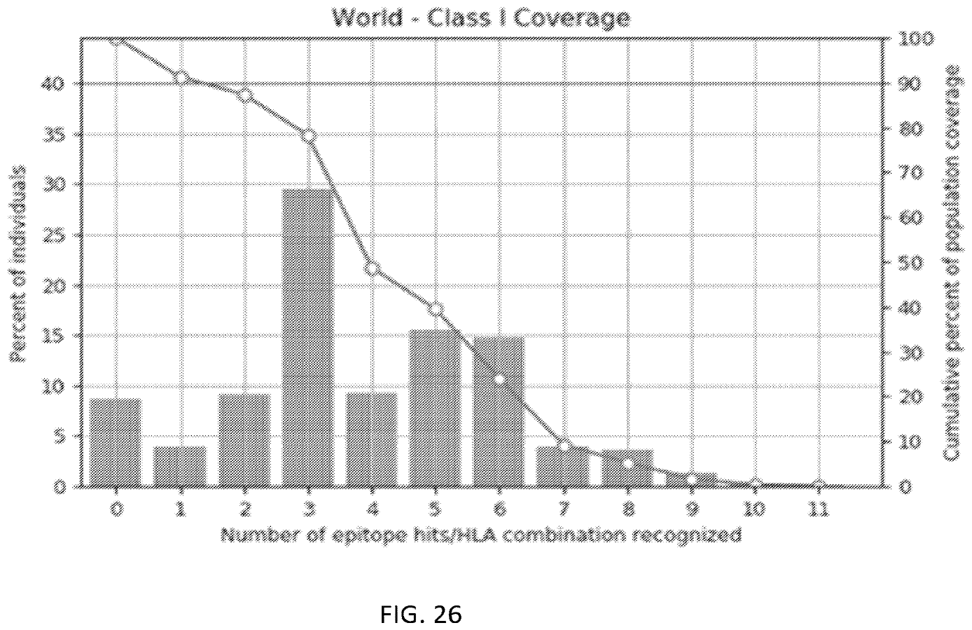

MHC I-restricted epitopes for TOP2A were designed through IEDB NetMHCpan EL 4.1, which returns epitopes along with their predicted binding affinity using two metrics for the top 27 expressed HLA alleles that covers >97% the human population. As shown in FIG. 25, regions with high affinity to more MHC-I alleles are indicated as the “hot” zone. We further computed other epitope specific clinical checkpoint parameters, such as immunogenicity, antigenicity, allergenicity, toxicity etc. using extra algorithms. The MHC-I epitopes we finally chose are listed in Table 3, which reaches 91% population coverage calculated by IEDB coverage tool (FIG. 26).

TABLE 3
MHC-I epitopes selected for TOP2A

| Peptide sequence | SEQ ID NO: |
|---|---|
| GINYREVTF | 20 |
| TVETASREY | 21 |
| SLKVIHEQV | 22 |
| VTKLVDVVK | 23 |
| SILNWVKFK | 24 |
| YLYGQTTTY | 25 |
| MTIINLAQN | 26 |
| VLFDHVGCL | 27 |

### Example 17—A Combination of TOP2A Vaccine with Those of Cyclin E2 and KIF15

The goal is to develop a multi-antigen preventive vaccine simultaneously targeting multiple TNBC tumor antigens (TOP2A, cyclin E2, and KIF15) to overcome potential antigen-negative variant escape. Previous human ductal carcinoma in situ (DCIS) vaccine studies have shown that targeting a single antigen (HER2) can induce target antigen loss in tumor cells. As antigen escape is the main potential mechanism for evading immunoprevention when targeting a single antigen, targeting multiple antigens may be an effective strategy to limit antigen-negative tumor escape.

Recently, we have explored two additional tumor antigens: cyclin E2 and KIF15. Overexpression of cyclin E2 has been detected in several human cancers including breast cancer, leukemia, lung cancer, ovarian cancer, and bladder cancer. Cyclin E2 MHC class I restricted peptide vaccines have also been identified and have been shown to elicit peptide-specific cytotoxic T lymphocytes targeting leukemia. KIF15 (kinesin family member 15) is overexpressed in multiple cancer types including breast cancer, pancreatic cancer, hepatocellular carcinoma, and lung cancer.

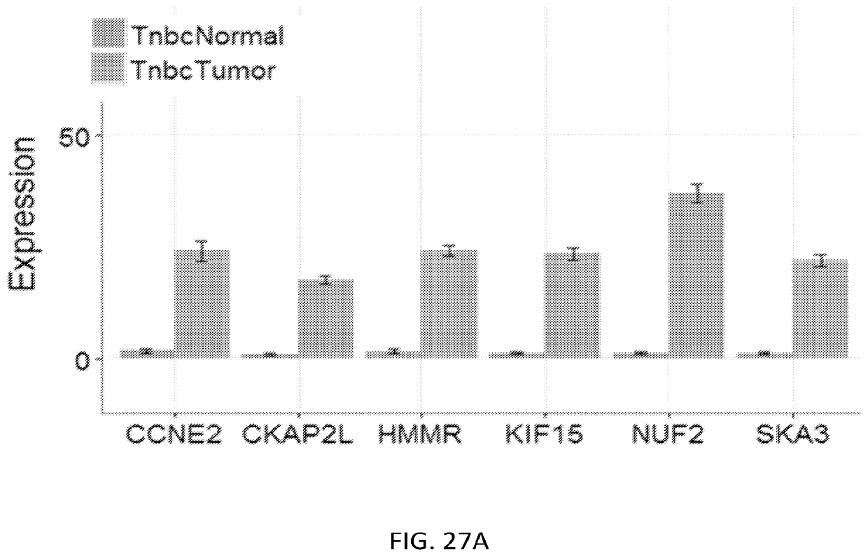

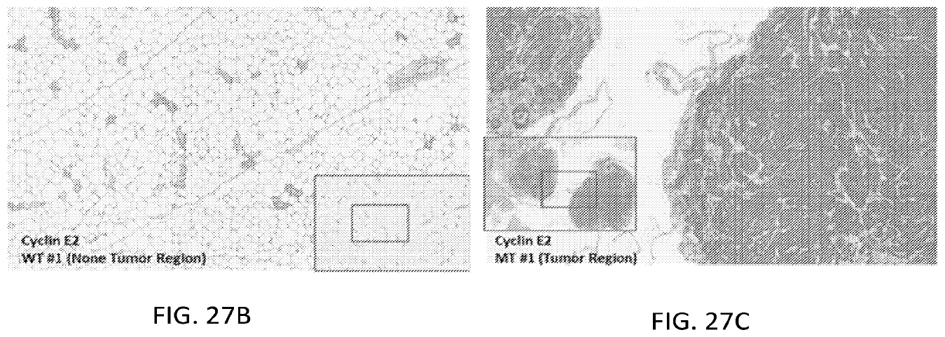

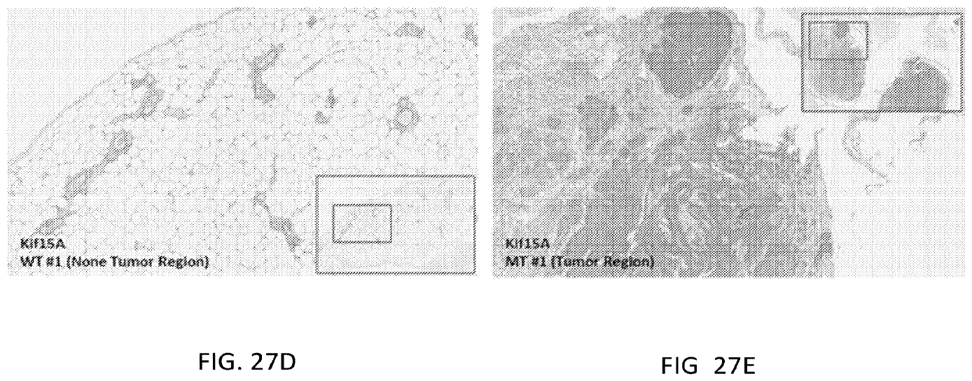

Overexpression of KIF15 in breast cancer is linked to tumor size, lymph node metastasis, advanced TNM stages and unfavorable prognosis. Both cyclin E2 and KIF15 are overexpressed in human TNBCs from the TCGA RNA-seq datasets (FIG. 27A) and overexpressed at the protein level using immunohistochemistry of TNBCs from C3 (1)/Tag mice (FIG. 27B-E).

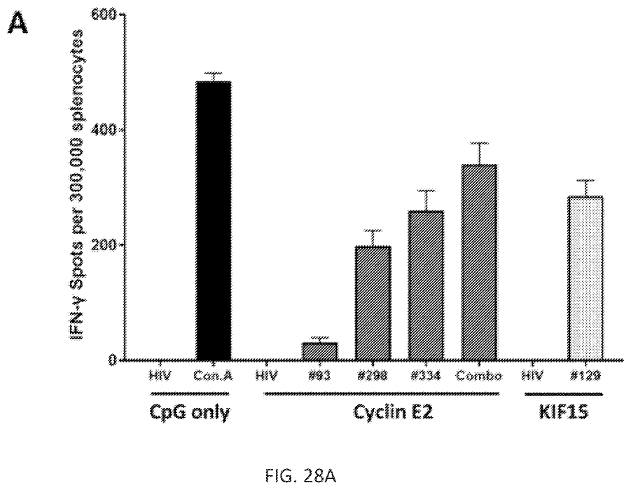

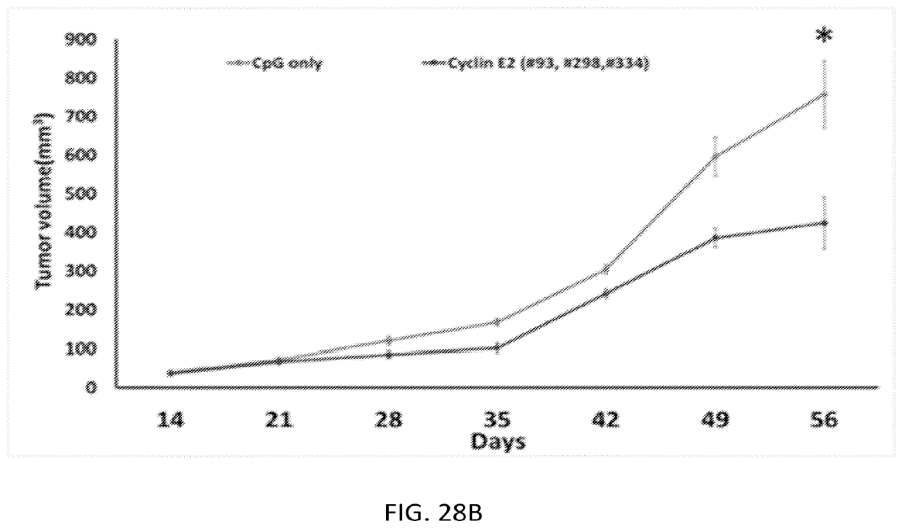

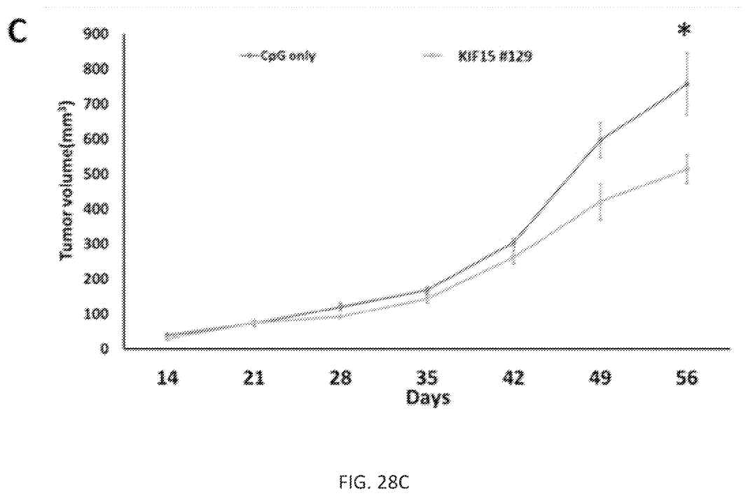

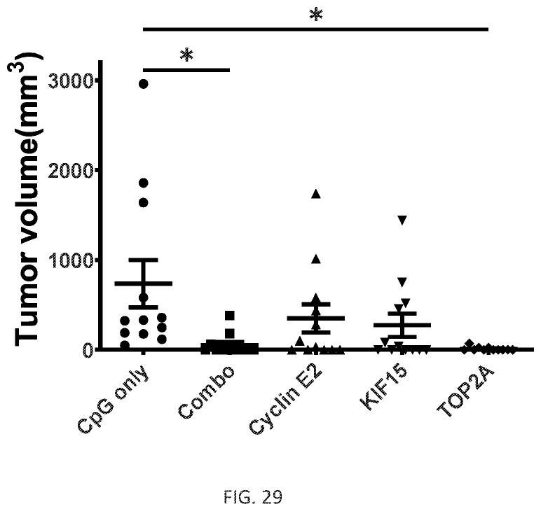

We developed both cyclin E2 and KIF15 MHC-II vaccines using the multi-scoring system to identify immunogenic epitopes (Table 4). Two cyclin E2-specific Th1 MHC class II-restricted epitopes (p298 & p334), which are highly homologous between species, elicited type I immunity in mice (FIG. 28A). After cyclin E2 vaccination, mammary tumor growth was significantly inhibited in the C3 (1)/Tag-RARE syngeneic mouse model (FIG. 28B). For KIF15, one KIF15-specific Th1 MHC class II-restricted epitope (p129) that is 93.3% homologous between species elicited type I immunity in mice (FIG. 28A and Table 4). After KIF15 vaccination, mammary tumor growth was significantly inhibited in the C3/(1) Tag mouse model (FIG. 28C). It is shown that the multi-antigen preventive vaccine targeting Top2A, cyclin E2, and KIF15 was more effective than targeting cyclin E2 or KIF15 alone (and as effective as Top2A vaccine alone) in the C3 (1)/Tag mouse model (FIG. 29). This multi-antigen preventive vaccine should also help overcome antigen-negative variant escape that has been observed with single antigen vaccines. Currently, a mRNA vaccine is also in development that contains the epitopes of TOP2A, cyclin E2 or KIF15 and will test its efficacy and immune response in relevant preclinical animal models.

TABLE 4
Peptide Sequences of the Cyclin E2 and KIF15

| Antigen | Peptide sequences | | |
|---|---|---|---|
| Cyclin E2 | #298 | AAALCHFTSIEVVKK | SEQ ID NO: 28 |
| | #334 | VVKSTSPVKLKTFKK | SEQ ID NO: 29 |
| KIF15 | #129 | SHNLRGVIPRSFEYL | SEQ ID NO: 30 |

### Example 18—Striking Efficacy of a Vaccine Targeting TOP2A for Triple Negative Breast Cancer Immunoprevention

Triple-negative breast cancer (TNBC) is an aggressive subtype of breast cancer that has a poor prognosis. TOP2A is a key enzyme in

DNA replication and is a therapeutic target for breast and other cancers. TOP2A-specific Th1-promoting epitopes with optimal binding affinity to MHC II were identified using a combined scoring system. The multi-peptide TOP2A vaccine elicited a robust immunologic response in immunized mice, as demonstrated by the significant production of Th1 cytokines from immunized animals' splenocytes stimulated in vitro with TOP2A peptides. Anti-tumor efficacy of the TOP2A vaccine was demonstrated in a syngeneic TNBC mouse model, in which pre-graft preventive vaccination was associated with significantly decreased tumor growth as compared to adjuvant control. In a genetically engineered mouse (GEM) model of TNBC, vaccinated animals demonstrated a significant reduction in tumor incidence and average tumor volume compared to adjuvant control. Finally, we examined TCR sequences in CD4 tumor Infiltrating lymphocytes (TIL) from vaccinated mice and found that the TIL contained TCR sequences specific to the three vaccine peptides. These data indicate that our newly developed multi-peptide TOP2A vaccine is highly immunogenic, elicits TILs with vaccine specific TCRs, and is highly effective in preventing and intercepting TNBC development and progression in vivo.

### Introduction

Breast cancer is the leading malignancy in women with 281,550 estimated new cases and 43,600 estimated deaths reported in 2023 in the United States¹. Approximately 20% of breast cancers are classified as triple-negative breast cancer (TNBC) as they do not express estrogen receptor (ER), progesterone receptor (PR) and human epidermal growth factor receptor 2 (HER2), encom-passing the molecular subtypes of basal-like and the more recently defined claudin-low group². TNBC is the subtype of breast cancer observed in women with BRCA-1 germline mutations. In the sporadic setting, TNBC is also associated with African Americans, younger age, higher grade and mitotic index, and more advanced stage at diagnosis³. Specific risk factors that correlate with TNBC include reproductive factors (pregnancy and multiple childbirths) and obesity. In TNBC patients, the 5-year survival rate is much lower than other types of breast cancer including ER⁺PR⁺HER2⁻ (Luminal A) and HER2⁺subtypes^{4,5} Luminal A tumors have highly effective targeted therapies, e.g. hormonal agents (tamoxifen, aromatase inhibitors), whereas HER2 breast cancers are treated with anti-HER2 antibodies or small molecule HER2-kinase inhibitors⁶. In contrast, early and intermediate stages of TNBC are routinely treated with standard cytotoxic chemotherapy resulting in strong initial regression in most patients; however, resistance to chemotherapy subsequently develops in most patients. With the current challenges in treating TNBC, new approaches are needed to improve clinical outcomes.

Cancer vaccines studied to date have primarily been examined in late-stage cancers, which provide limited efficacy most likely due to the immune suppressive tumor microenvironment intrinsic to advanced malignancies. Immune suppression can involve multiple mediators including T regulatory cells (Treg), inhibitory macrophages and other suppressive factors. One way to potentially use cancer vaccines more effectively is in the prevention or early interception setting. If the antigens are expressed early in oncogenesis, vaccines could have utility in prevention. Several reports have now provided proof-of-concept to support the use of peptide vaccines targeting overexpressed self-antigens for cancer immunoprevention⁷⁻¹⁰. Molecular analysis of tumors has identified many genes that are overexpressed in breast cancer, which can be exploited as tumor antigens and potential vaccine candidates. Currently, the most common tumor antigens used in cancer immunotherapy are upregulated self-proteins, such as HER2. Vaccination with peptides targeting overexpressed HER2 in humans has been shown to be effective and well-tolerated^{11,12}. While mutated epitopes are recognized as foreign “neo-antigens” by the immune system, eliciting a type 1 immune response, epitopes derived from non-mutated self-antigens are more likely to trigger T helper 2 (Th2) cytokines such as interleukin (IL)-10 and IL-4 that can inhibit cytotoxic T-lymphocyte (CTL) proliferation and function. Recently, attempts have been made to specifically identify Th1-selective epitopes from non-mutated self-antigens that can elicit a Th1 response, when used in a vaccine, can elicit unopposed type 1 immunity and can be effective in preventing cancer growth in preclinical models. If Th2-inducing epitopes from the same protein are included in a vaccine, Th2 cells elicited by immunization may abrogate the Th1-mediated anti-tumor effect and it is imperative to remove Th2 epitopes from cancer 12,13

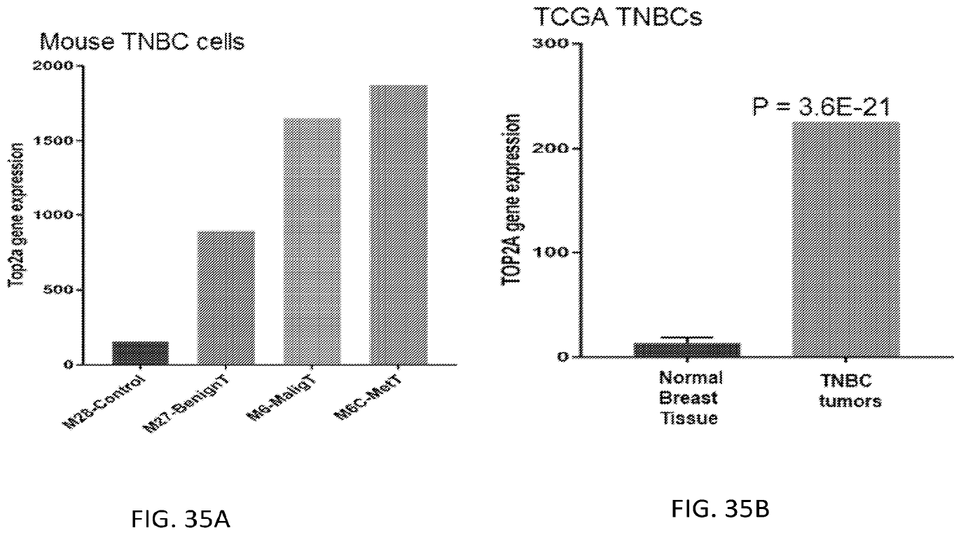

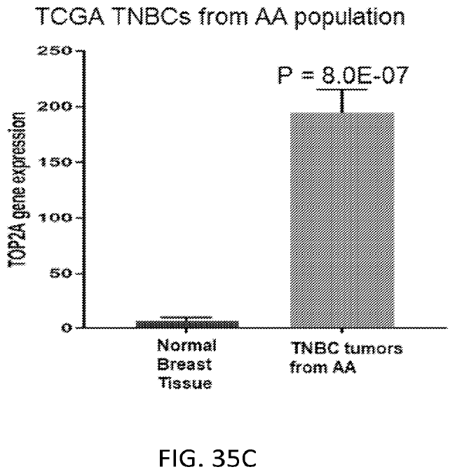

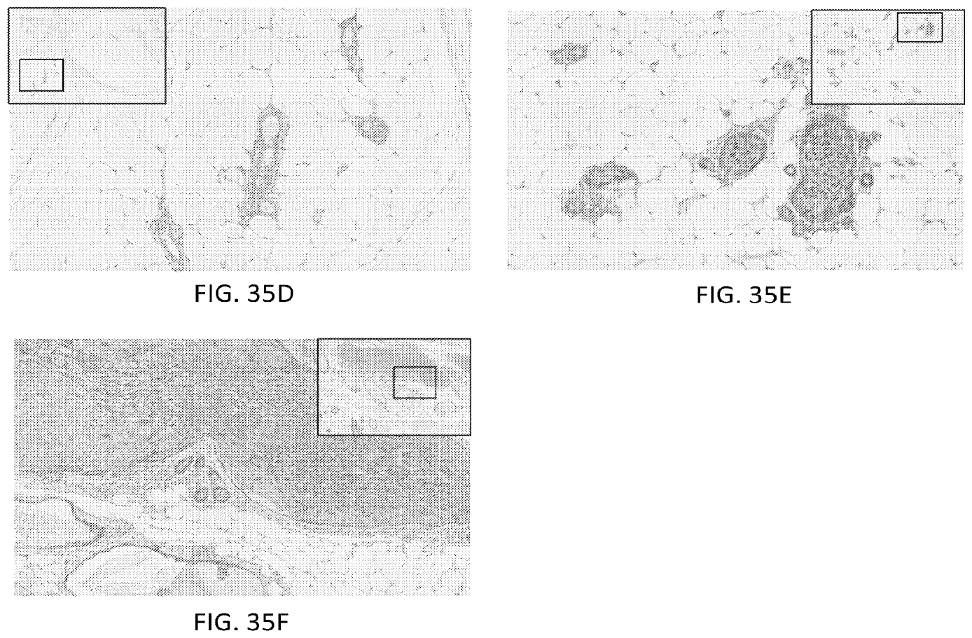

In the current study, we used The Cancer Genome Atlas (TCGA) databases and transcriptomic and/or proteomic analysis of normal vs. malignant human breast tissues to identify highly expressed genes in the malignant tissues. We found that topoisomerase 2 alpha (TOP2A) is highly expressed in human TNBC (FIGS. 35A-35F). TOP2A is a known key enzyme in DNA replication, cancer cell proliferation and a direct or indirect target of several cytotoxic anticancer agents (e.g. anthracyclines and etoposides). Recent studies suggested that TOP2A has potential applications in breast cancer detection and management¹⁴. We constructed a TOP2A multi-peptide vaccine and evaluated its immunogenicity and preventive efficacy against TNBC in the C3 (1)/Tag mouse model, which has been utilized extensively because of its genetic similarity to the human basal subtype of TNBC^{15,16}. Both C3 (1)/Tag TNBCs and mammary carcinoma cell lines (derived from C3 (1)/Tag mammary tumors) implanted into the mammary fat pads have been reported to spontaneously metastasize to the lung and liver¹⁷. By using single-cell RNA sequencing (scRNAseq) analyses we show that TOP2A multi-peptide vaccination induces anti-tumor CD4+Th1 cells and cytotoxic CD8+ T cells in mouse breast tumor and lymph node tissue samples. Additionally, we have found that the TOP2A vaccine induces a potent TOP2A-specific memory immune response that rejected secondary tumor challenge. Finally, we examined TCR sequences from CD4 tumor infiltrating lymphocytes (TIL) in tumors from vaccinated mice and detected TILs with TCR sequences against all the immunizing peptides. Taken together, our data demonstrate that the multi-peptide TOP2A vaccine is highly immunogenic and efficacious in the prevention of TNBC and warrants further investigation.

### Results

### Gene Expression Analysis of TOP2A

It was found that the TOP2A gene was overexpressed in various mouse breast tumor cell lines and human TNBC and lung tumors (FIGS. 35A-35F). In mice, the TOP2A gene was overexpressed in mouse mammary tumor cell lines M27 (mammary benign tumor cell line), M6 (mammary malignant tumor cell line), and M6C (mammary metastatic tumor cell line), with the M28 mammary cell line serving as a normal control (FIG. 35A). The TOP2A gene was also highly overexpressed in TCGA TNBC samples (12 normal breast tissues and 137 tumor samples of the TNBC patients) with limited expression in normal mammary tissues (FIG. 35B). We also performed expression analysis of TNBC in African Americans (AA), who are at high risk for TNBC. Similar to the general TNBC population, TOP2A was significantly overexpressed in TNBC from the AA subpopulation (FIG. 35C). Representative images of TOP2A IHC staining are shown in (FIGS. 35D-35F). TOP2A was highly expressed on ductal carcinoma in situ (DCIS) and malignant tumor tissue from C3 (1)/Tag mice (FIGS. 35E and 35F). However, in normal mammary tissue from wild-type mice, TOP2A was not detected (FIG. 35D). These data indicate that the TOP2A gene is overexpressed in TNBCs isolated from both mice and humans.

### Identification of Th1 TOP2A Epitopes for Vaccine Design

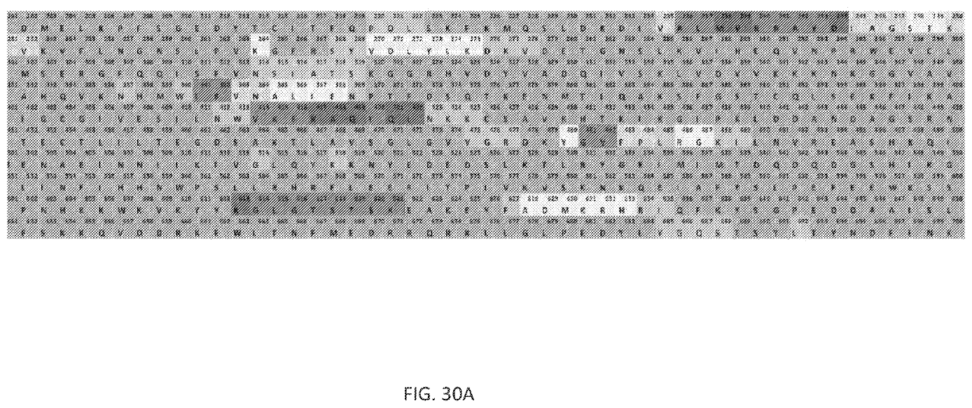

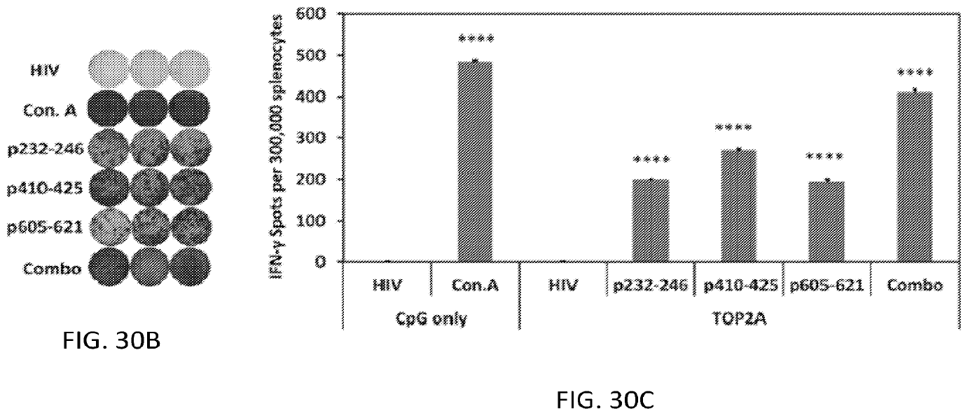

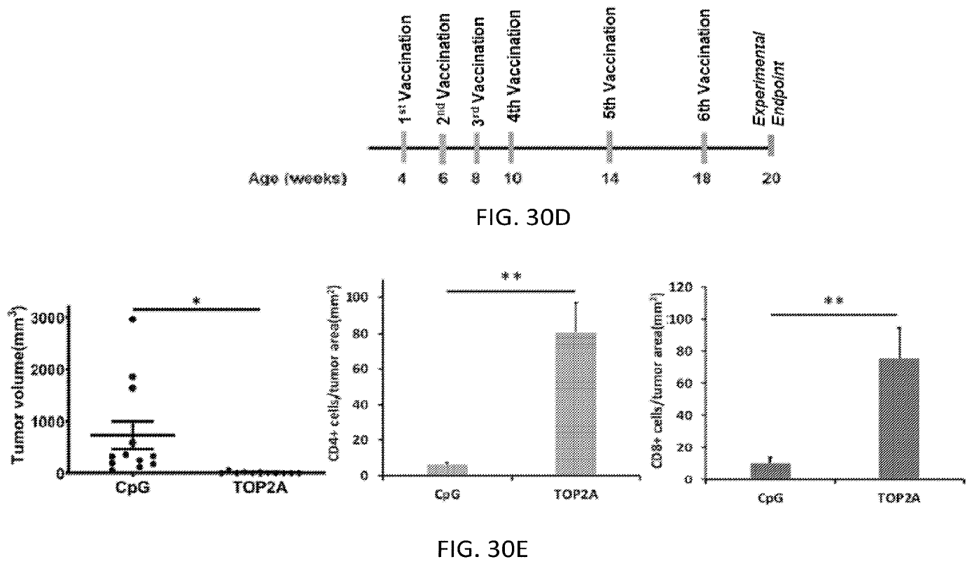

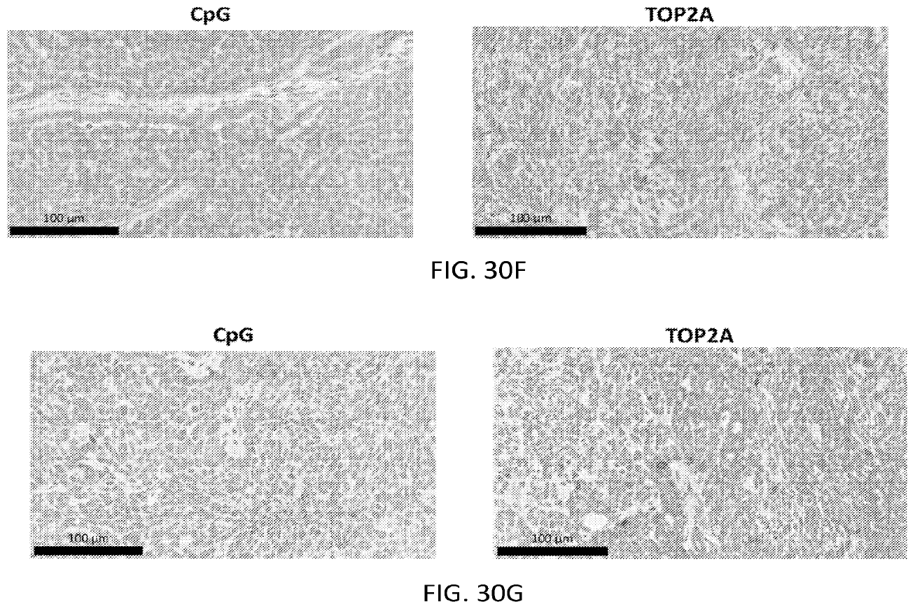

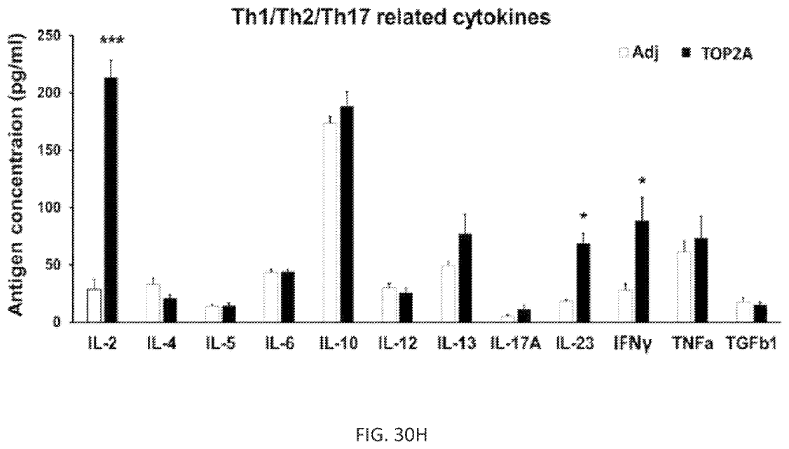

Using a multi-scoring system that combines multiple MHC class II peptide binding algorithms, we identified and selected 3 Th1-promoting vaccine candidate peptides, p232 (p232-p246), p410 (p410-p425), and p604 (p605-p621) (FIG. 30A). Immunogenicity of the TOP2A peptides was evaluated in C3 (1)/Tag-REAR mice via IFN-γ ELISpot assays (FIG. 30C). Splenocytes from TOP2A vaccinated mice demonstrated strong immune responses with a mean for IFN-γ secreting cells of around 400 spots per well (SPW) compared to less than 10 SPW for negative control HIV peptides. Additionally, we examined the immunogenicity of all three peptides combined (combo). Interestingly, combo-vaccinated mice yielded a significantly stronger immune response than single peptide-vaccinated mice. Among three peptides of TOP2A, two murine peptide sequences (p232-246 and p605-621) exhibited 100% sequence identity with the human TOP2A sequence, while another peptide sequence (p410-425) showed a 93% similarity. These peptides p232, p410, and p604 were chosen to formulate a multi-peptide TOP2A vaccine for the preventive efficacy study.

### TOP2A Vaccination Prevented TNBC Development in a Genetically Engineered Mouse Model

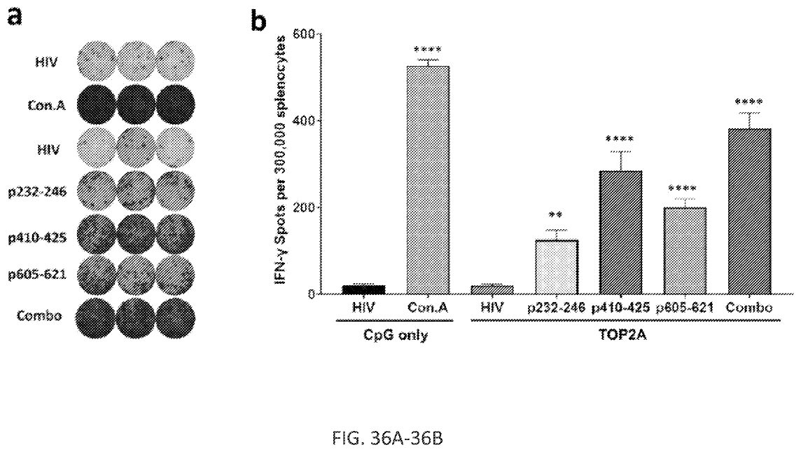

To examine tumor preventive effects of the TOP2A vaccine in a more clinically relevant model, we employed the C3 (1)/Tag transgenic mouse model which develops invasive mammary gland tumors that share important molecular and biologic features with human basal-like TNBC¹⁷. The experimental design for these experiments is shown in FIG. 30D. Briefly, mice were vaccinated once every two weeks for four consecutive weeks, and then monthly at 14- and 18-weeks of age. We measured palpable tumor volumes at the end of the experiments (20 weeks). As shown in FIG. 30E, the TOP2A vaccination significantly reduced tumor volume as compared to the adjuvant control (CpG only) with an average tumor size of 736 mm³ in adjuvant controls vs. 11 mm³ in vaccinated mice (p<0.05). Notably, the vaccine completely prevented mammary gland tumor growth in 8 of the 11 TOP2A vaccinated mice, while all adjuvant controls developed tumors. We confirmed the immunogenicity of TOP2A peptides via the IFN-γ ELISpot assays (FIGS. 36A and 36B). We evaluated the number of CD4+ and CD8+ TIL per mm2 tumor area in those animals that developed tumors (3 TOP2A vaccinated and 12 CpG control mice) and observed that TOP2A vaccination significantly increased CD4+ and CD8+ TIL over adjuvant controls (FIG. 30F, g; p<0.01). We closely monitored all animals in all groups and body weights, serum ALT and AST levels were not significantly changed in 20 week-age old (FIGS. 301-30K). These results demonstrate that TOP2A vaccination can effectively prevent TNBC development in this genetically engineered mouse model and suggest that vaccine-induced tumor antigen-specific T cell responses play a critical role in the immunopreventive effect of the vaccine.

### TOP2A Vaccination Induced a Th1 Cytokine Response

To evaluate Th1 and Th2 cytokine production in response to the TOP2A vaccine, we measured the production of 12 cytokines from in vitro peptide-stimulated splenocytes that had been isolated from vaccinated C3 (1)/Tag GEM mice (FIG. 30H). The most abundant cytokines detected in response to the TOP2A peptide pool stimulation were Th1 cytokines, IL-2 and IFN-γ, increased approximately 7- and 3-fold, respectively, as compared to adjuvant controls. In contrast, there was no notable increase in Th2 cytokine production (IL-4, IL-5 and IL-13) as compared to the adjuvant controls. Interestingly, the TOP2A vaccine stimulated not only Th1 cytokine production (IL-2 and IFN-γ) but also IL-23 which promotes the differentiation of Th17 lymphocytes. These data suggest that Th1 and Th17 immune responses are predominantly elicited by the TOP2A-specific peptide vaccine.

### TOP2A Multi-Peptide Vaccination Slowed the Growth of Syngeneic Mouse TNBC Tumors

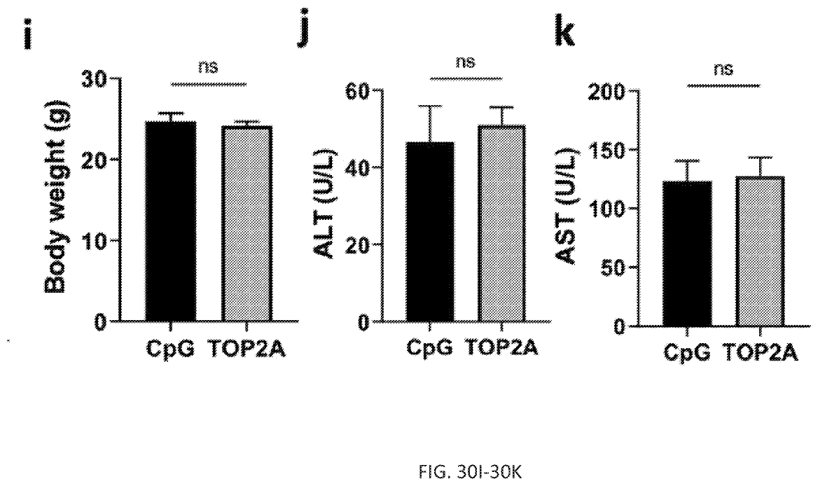

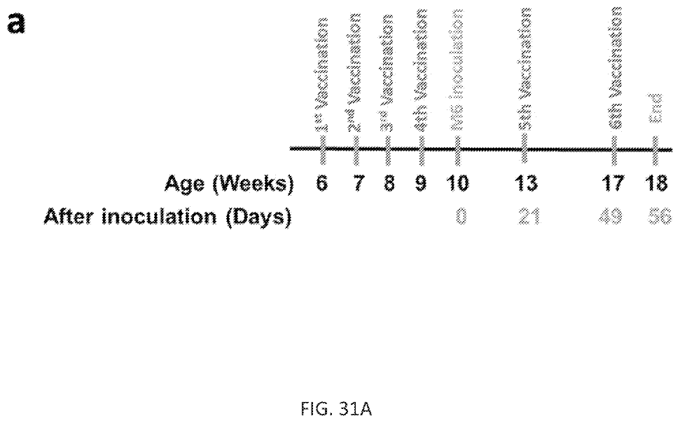

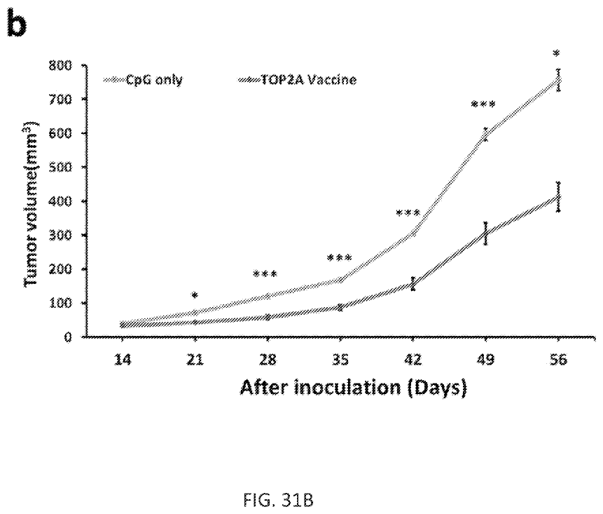

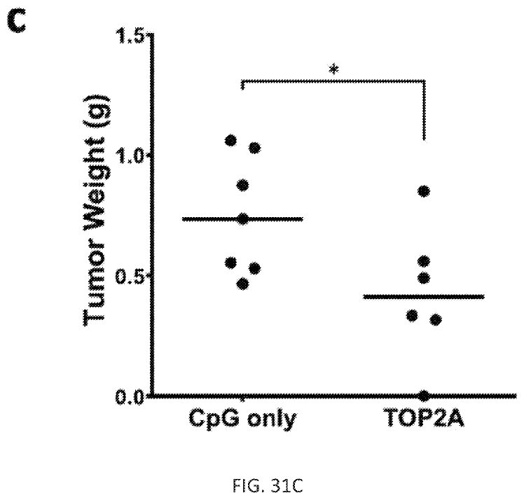

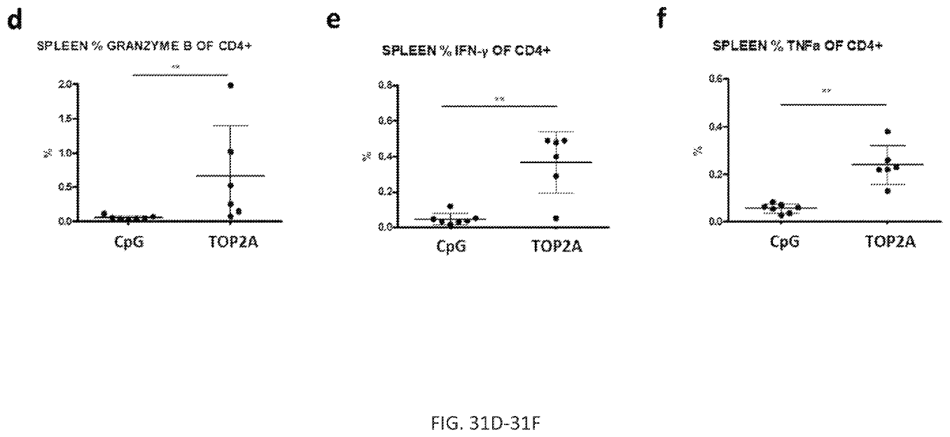

We next examined the antitumor efficacy of TOP2A vaccination in a syngeneic TNBC mouse model, C3 (1)/Tag-REAR mice inoculated with M6 tumor cells. As illustrated in FIG. 31A, 7-week old C3 (1)/Tag-REAR mice were given an initial vaccination with TOP2A peptides with CpG oligodeoxynucleotide (CpG ODN) adjuvant, followed by three more vaccinations at one-week intervals. One week after the last vaccination, M6 cells were implanted into #4 mammary fat pads of the C3 (1)/Tag-REAR mice. Additional vaccinations were administered at four-week intervals until the experimental endpoint. Notably, TOP2A vaccination significantly decreased tumor volumes as compared to CpG-only treated control mice (FIG. 31B; p<0.05). Average tumor size at the experimental endpoint was 757.2 mm³ in control vs. 413 mm³ in vaccinated animals. TOP2A vaccine also reduced tumor weight by nearly 40% (FIG. 31C, p<0.05). TOP2A vaccinated mice were also examined for the presence of a systemic immune response. Splenocytes collected from TOP2A vaccinated mice were stained for the intracellular markers (granzyme B, IFN-γ and TNFα) and analyzed by flow cytometry. Significant increases in CD4+ and CD8+ T cells expressing granzyme B, IFN-γ, and TNFα were observed in the spleens of TOP2A vaccinated animals as compared to controls treated with CpG only (FIG. 31D-31I). Taken together, these results indicate that the TOP2A peptide vaccine induced both local and systemic type 1 immune responses.

TOP2A Vaccinated C3 (1)/Tag Mice were Protected from the M6 Cell Secondary Challenge

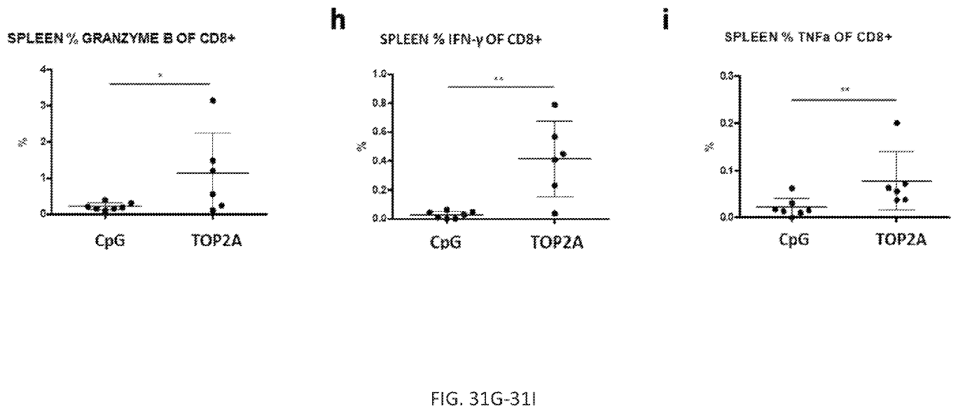

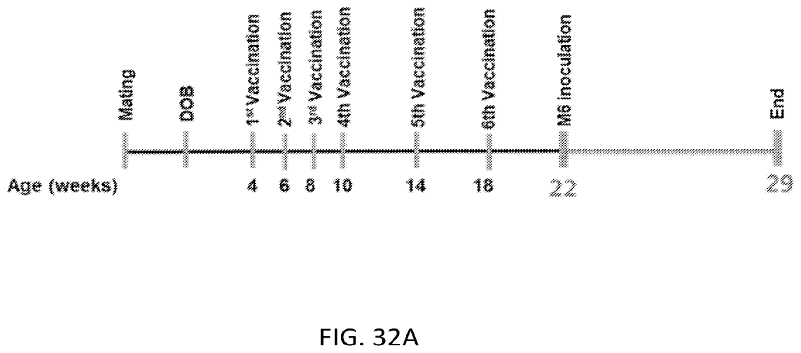

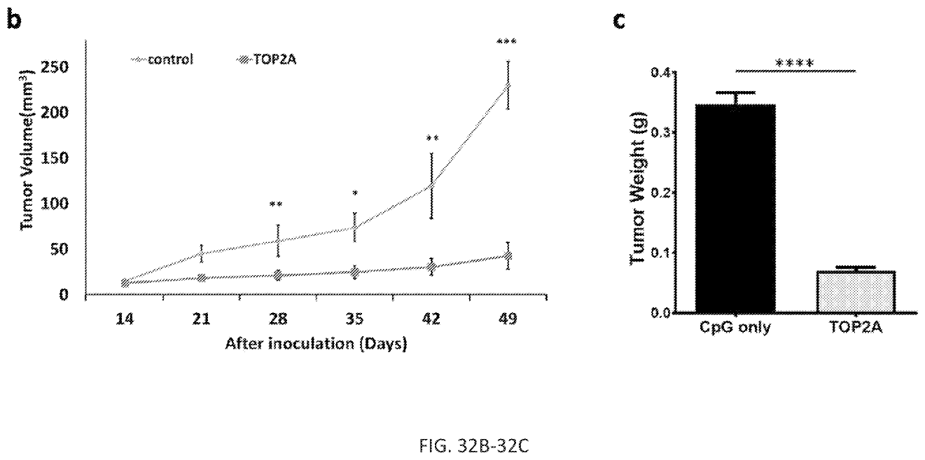

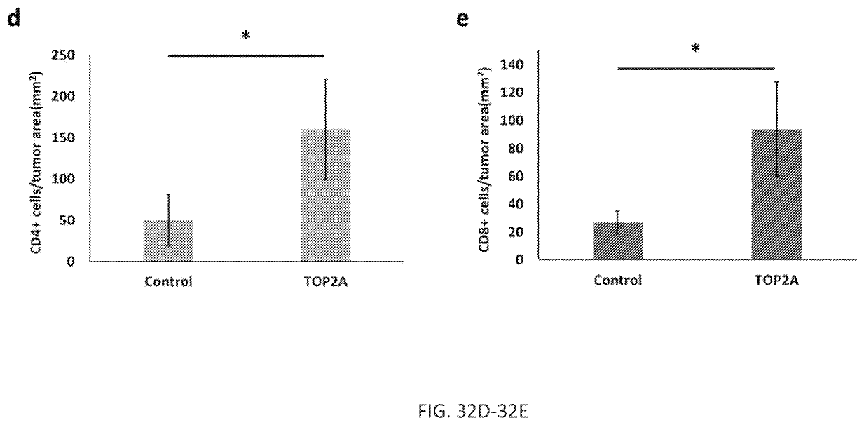

We tested the long-term immune memory protective effects of the TOP2A vaccine against secondary tumor cell development. The first “challenge” was the spontaneous tumor that develops in C3/Tag mice (see FIG. 32A for experimental design). Secondary tumor challenge involved transplanting the C3/Tag mice at 22 weeks of age (4 weeks after the final vaccine) with syngeneic M6 tumor cells. Notably, TOP2A vaccinated mice did not develop spontaneous tumors, and the vaccinated mice had significantly smaller M6 TNBC tumors at the experimental endpoint (FIGS. 32B and 32C). Average tumor size in the vaccinated mice as compared to the adjuvant controls was 42 mm³ vs. 229.9 mm3, respectively (FIG. 32B; p<0.001). In TOP2A vaccinated mice, tumor weight in the TOP2A vaccinated mice was also significantly reduced by nearly 85% (FIG. 32C). The numbers of CD4+ and CD8+ TIL in TOP2A vaccinated mice were also significantly increased, as shown in FIG. 32D. These results suggest that the TOP2A vaccine induces long-term T-cell memory to help resist tumor re-emergence.

### Single Cell Gene Expression Landscape in Breast Tumor and Lymph Node Tissues

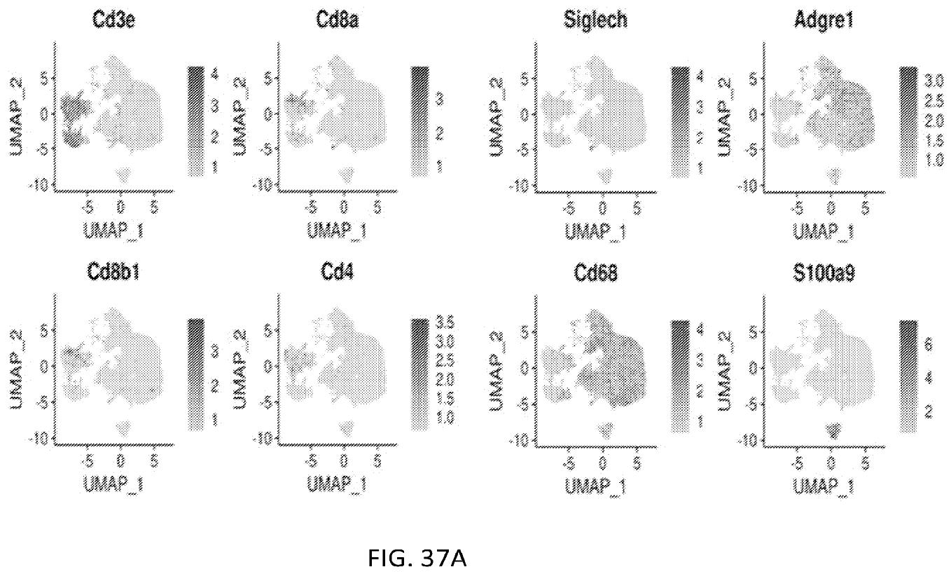

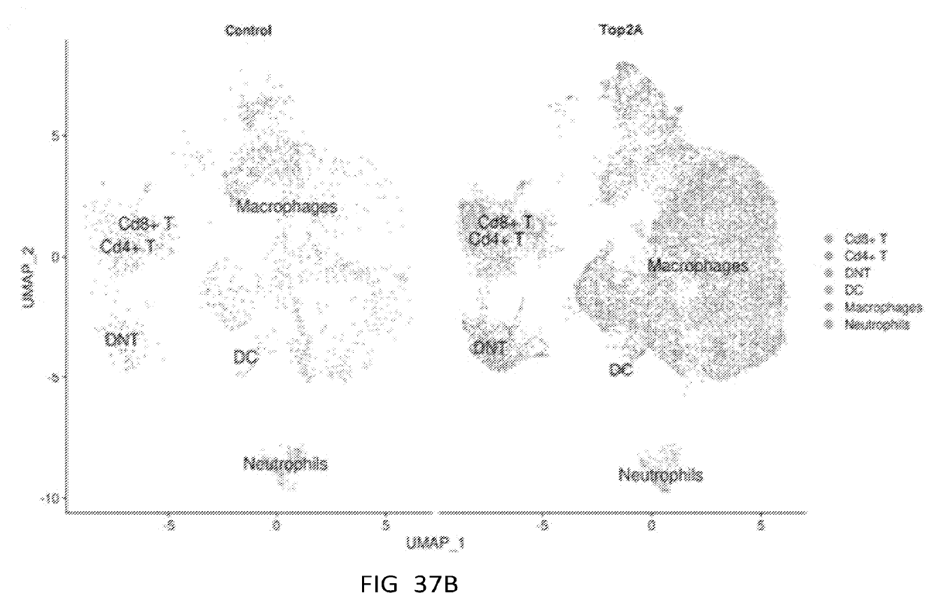

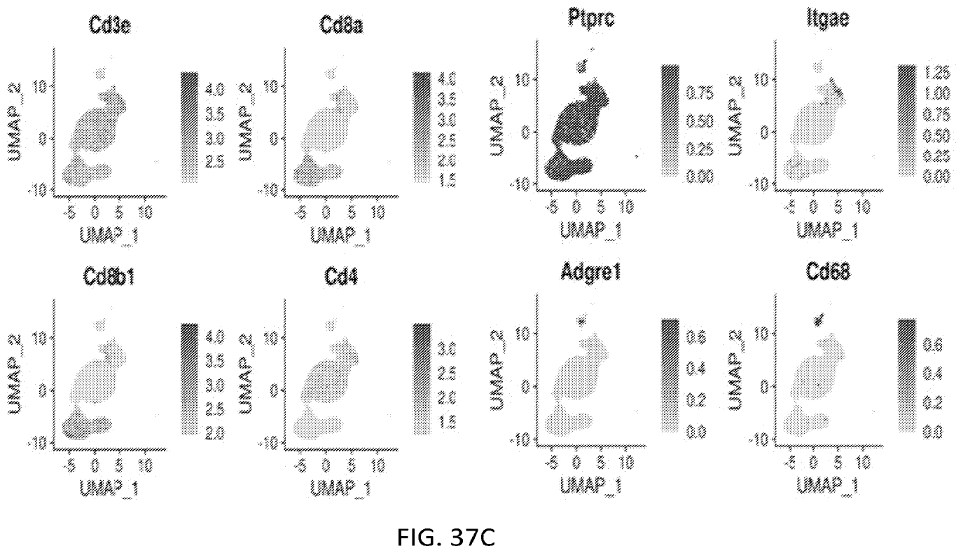

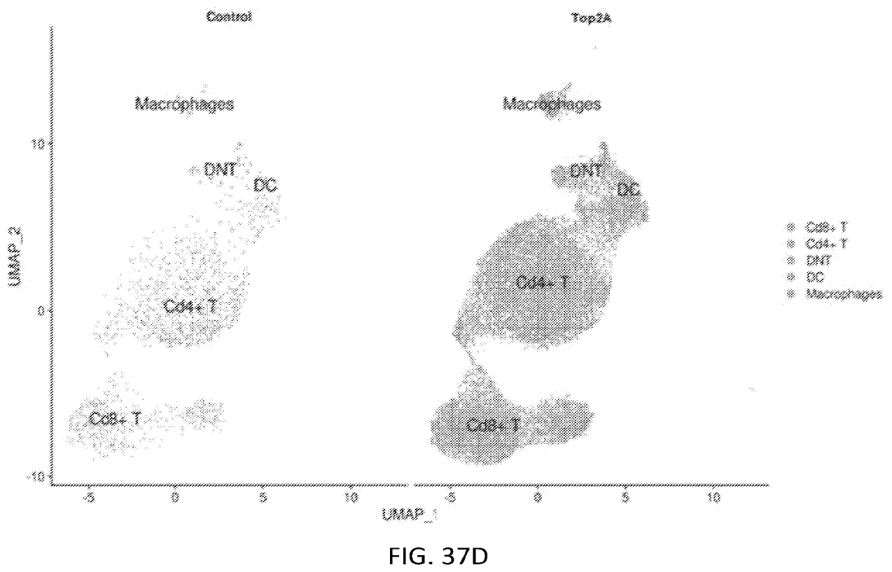

We utilized the Seurat package to perform fine clustering of sequenced single cells from mouse breast tumor and lymph node tissues^{18,19}. Gene expression data of single cells were aligned and projected in 2-dimensions using uniform manifold approximation and projection (UMAP)²⁰. The gene expression patterns of canonical markers were analyzed to characterize different types of immune cell populations in the tumor samples. Six immune cell populations were detected in the breast tumor samples including CD8+ T cells, CD4+ T cells, CD4/CD8 double-negative T cells (DNT), dendritic cells (DC), macrophages and neutrophils (FIGS. 37A and 37B). Single cell expression data from the lymph node tissues was also projected by UMAP and immune cell populations identified included CD8+ T cells, CD4+ T cells, DNT, DC and macrophages (FIGS. 37C and 37D).

### TOP2A Vaccine Treatment Increased the Proportions of Tumor Specific Cytotoxic CD8+ T Cells in Mouse Breast Tumor and Lymph Node Tissues

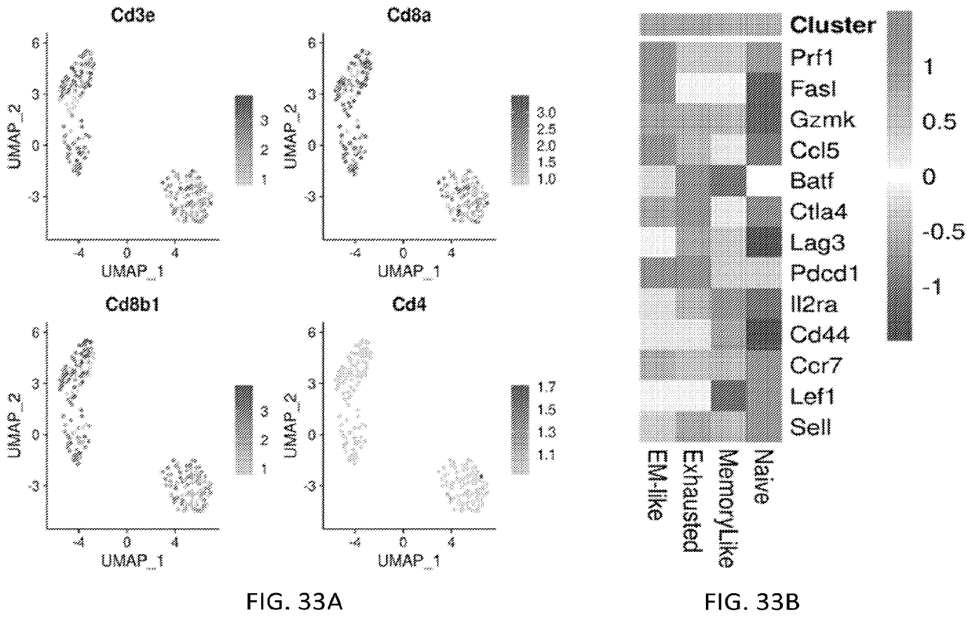

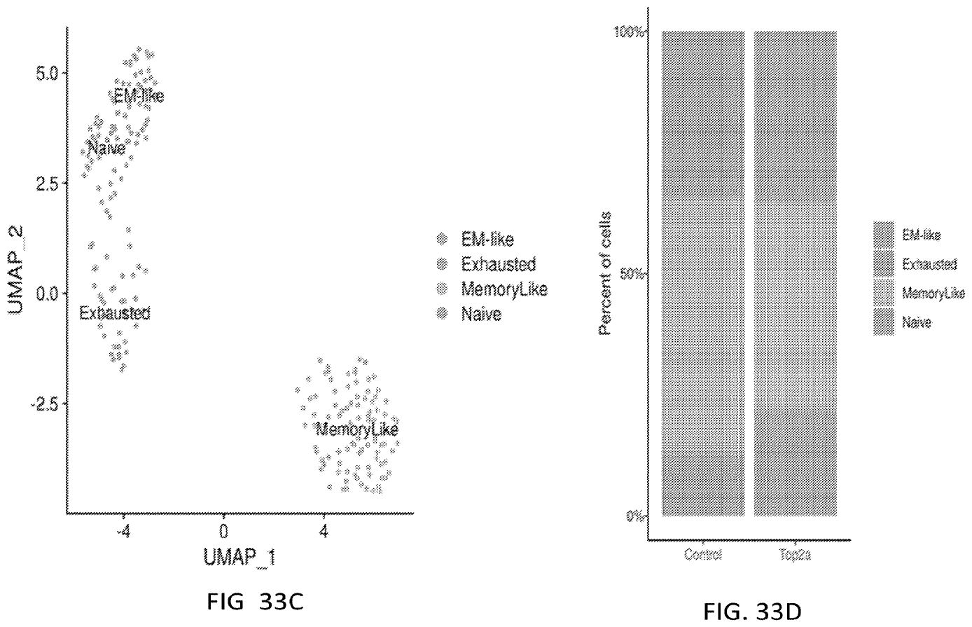

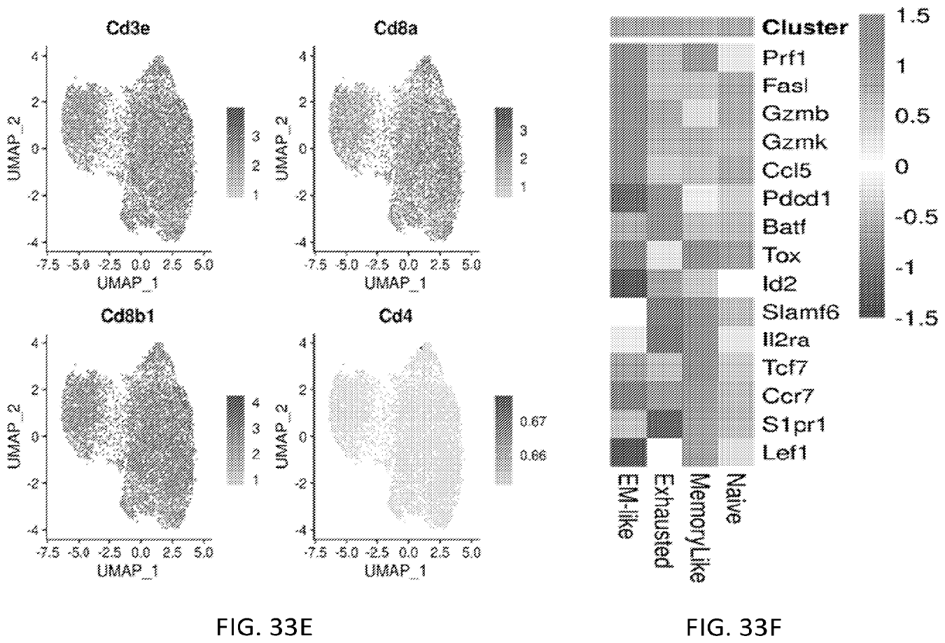

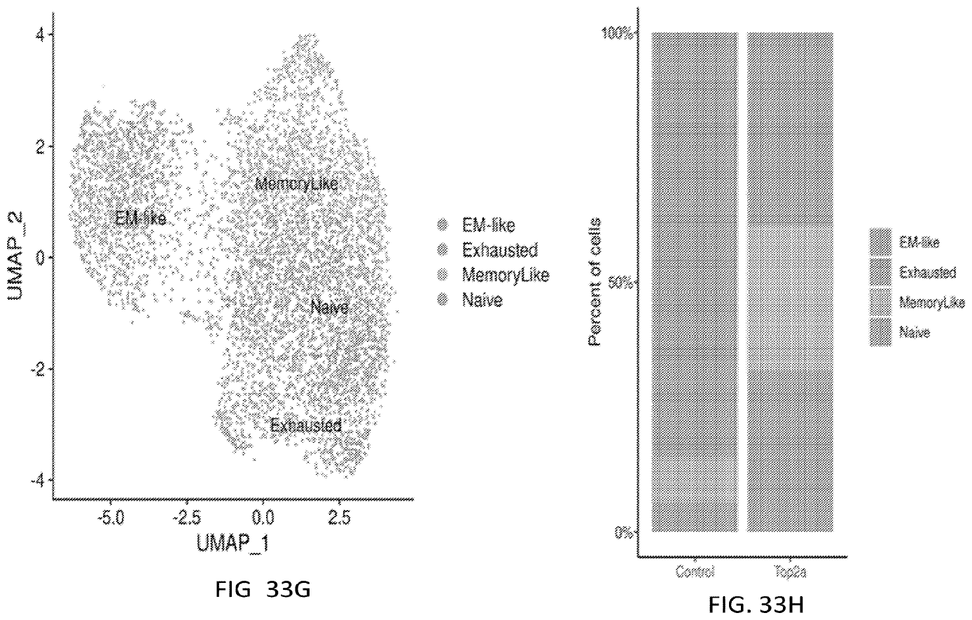

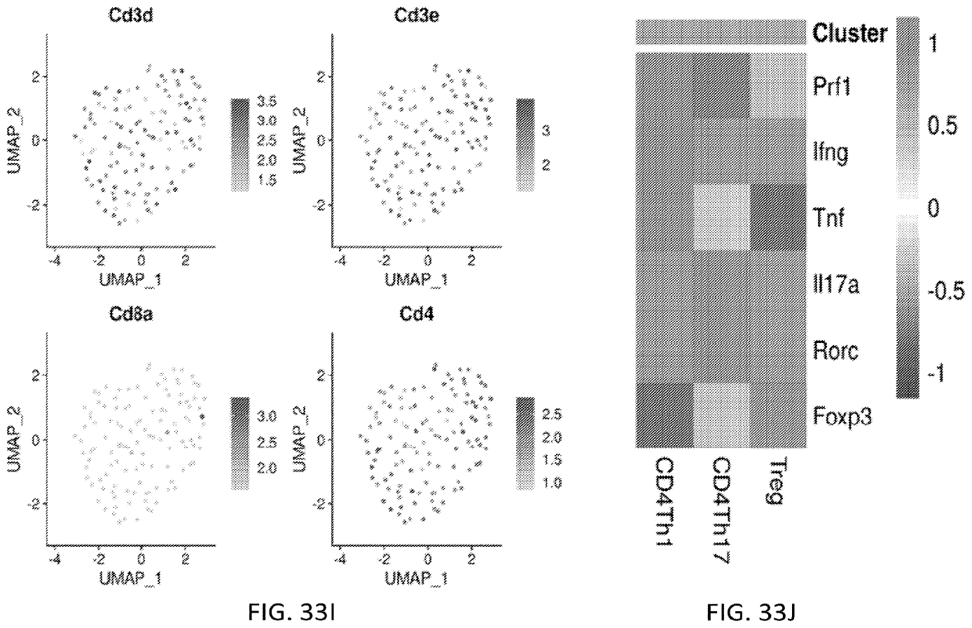

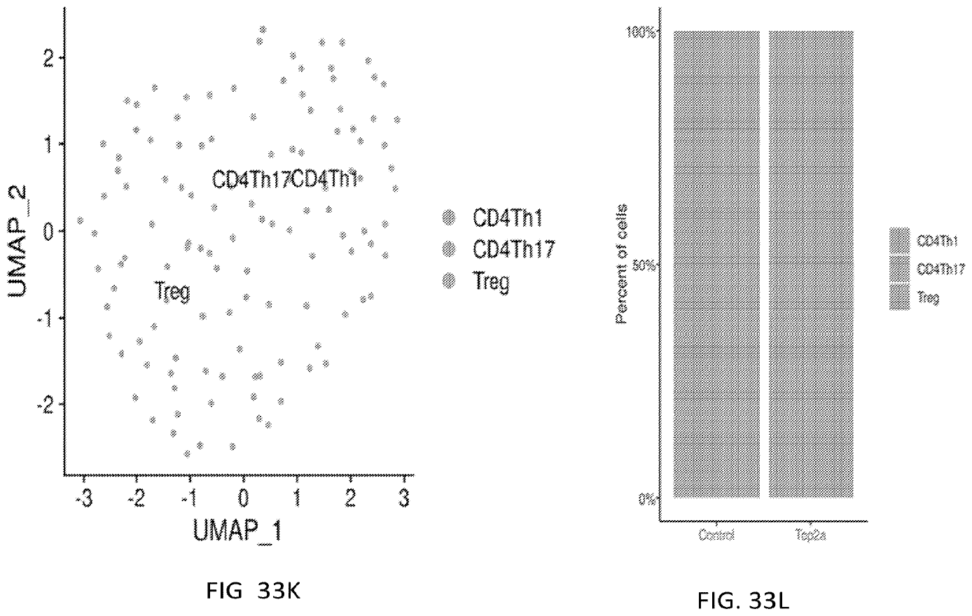

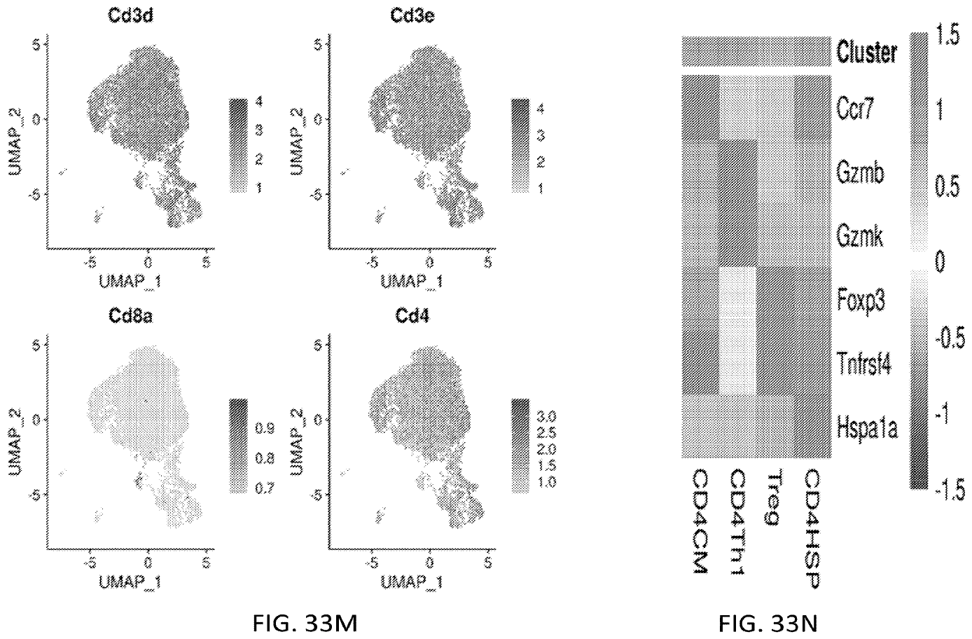

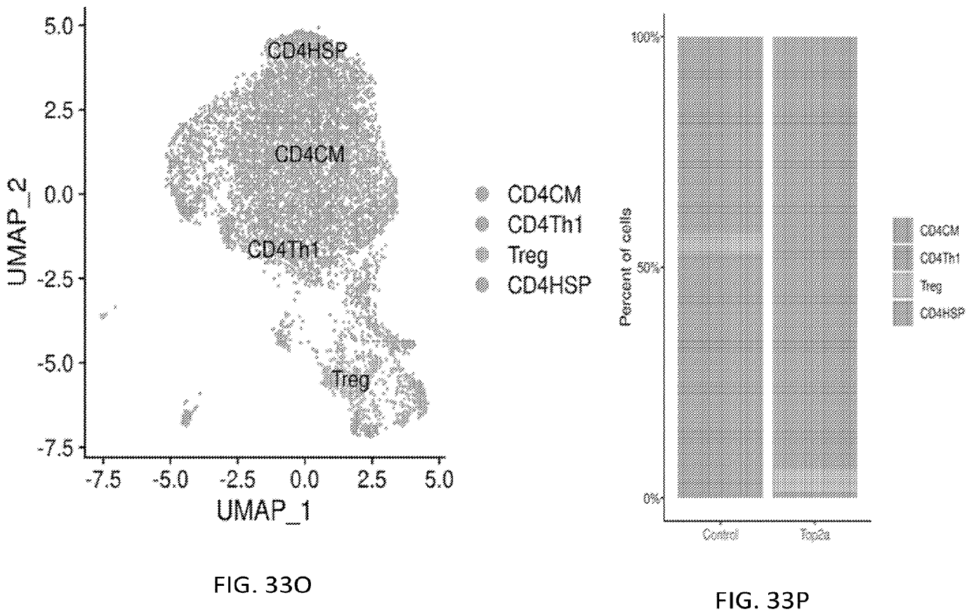

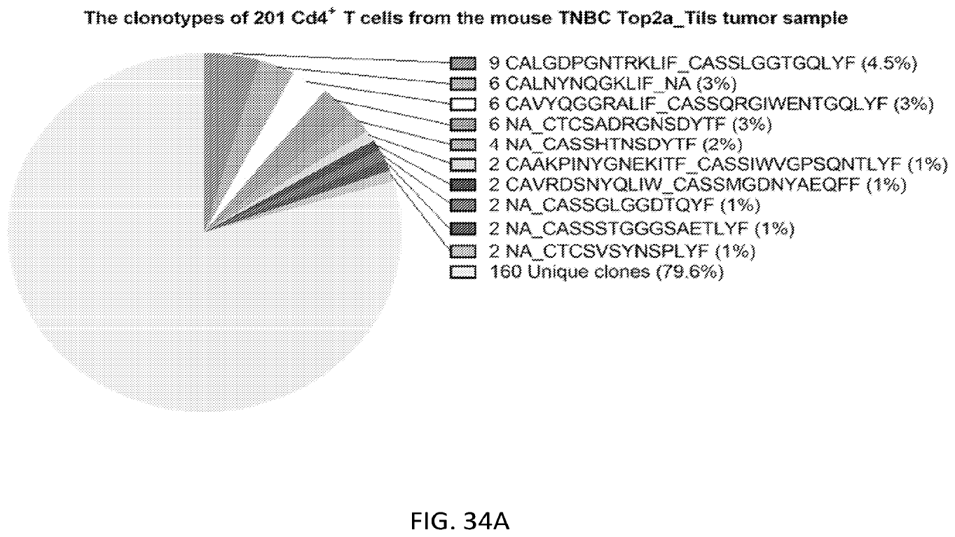

To determine the effects of TOP2A vaccine treatment on different T cell subsets, we performed deep clustering of CD8+ T cells, CD4+ T cells and DNT cells from mouse breast tumors of 3 of 11 mice that had tumors (8 mice were tumor-free). The location of the CD8+ T cells was identified by the canonical markers (FIG. 33A). Unsupervised clustering of CD8+ T cells using the TILPRED program (https://github.com/carmonalab/TILPRED) identified four CD8+ T cell subsets with distinct transcriptomic profiles (FIGS. 33B and 33C) 21 The CD8 subsets included effector-memory (EM)-like, exhausted, memory-like, and naïve CD8+ T cells. In cancer, the EM-like and exhausted CD8+ anti-tumor and pro-tumor functions, respectively. TOP2A vaccination greatly increased the abundance of the anti-tumor EM-like CD8+ T cells in the breast tumor microenvironment (TME) (FIG. 33D). In contrast, the exhausted CD8+ T cells were decreased by the TOP2A vaccine treatment. These data suggest that the TOP2A vaccine treatment improves the overall composition of beneficial anti-tumor CD8+ T cells in breast tumors. In the lymph node samples from tumor-bearing mice, we observed similar effects of TOP2A vaccine treatment on CD8+ T cell subsets. CD8+ T cells from the lymph node samples were plotted (FIG. 33E), and CD8+ T cell subsets were identified using corresponding markers (FIGS. 33F and 33G). TOP2A vaccine treatment increased the abundance of EM-like CD8+ T cells and greatly reduced the proportion of exhausted CD8+ T cells in the lymph nodes (FIG. 33H).

TOP2A vaccine treatment increased the abundance of CD4+Th1 cells in mouse breast tumor and lymph node tissues

Functional CD4+ cells could play a major role in the anti-tumor response induced by TOP2A vaccine treatment. We analyzed CD4+ T cells from the breast tumors and identified three types of CD4+ T cell subsets including CD4 Th1, CD4 Th17 and Treg cells (FIG. 33I-33K). CD4+Th1 cells overexpressed the marker genes perforin, IFNγ, and TNF; CD4 Th17 T cells overexpressed IL17a and Rorc; Treg cells overexpressed Foxp3 (FIG. 33J). TOP2A vaccine treatment significantly increased the proportion of CD4+Th1 and Th17 cells in breast tumors while decreasing the abundance of Treg cells (FIG. 33I), suggesting that the anti-tumor function of CD4+ T cells was significantly enhanced by TOP2A vaccine treatment. In the lymph node samples, four types of CD4+ T cells subsets were identified: CD4 CM (central-memory T cells), CD4 Th1, Treg and CD4HSP (CD4+ T cells overexpressing Hspa1a) (FIG. 33M-330). Similar to the breast tumor samples, we observed increased frequencies of CD4+Th1 cells after TOP2A vaccination (FIG. 33I). CD4 CM cells were also increased, while immune inhibitory CD4 HSP cells were decreased by the TOP2A vaccine treatment (FIG. 33P)²². Our results suggest that CD4+Th1 cells are involved in the anti-tumor rated by the TOP2A vaccine.

### TOP2A Vaccine Treatment Also Impacts Other Immune Cells in Mouse Tumor and Lymph Node Tissues

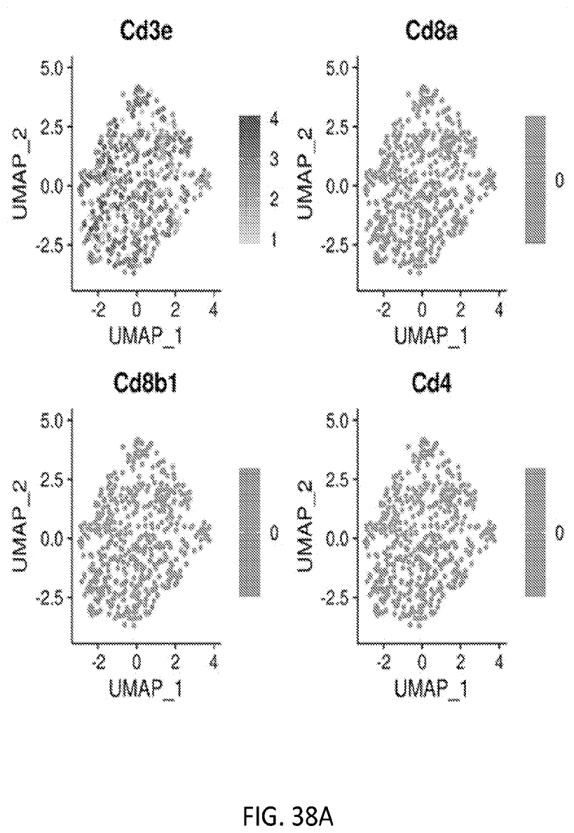

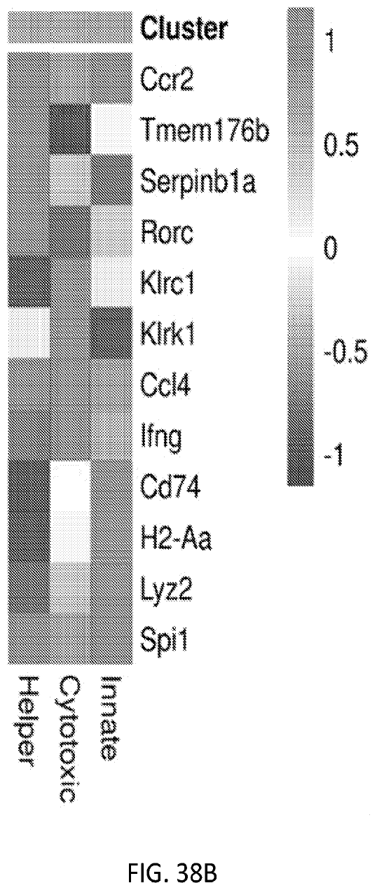

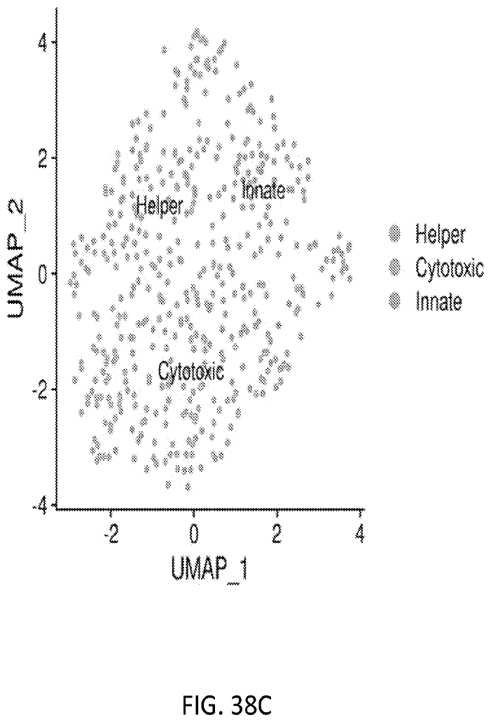

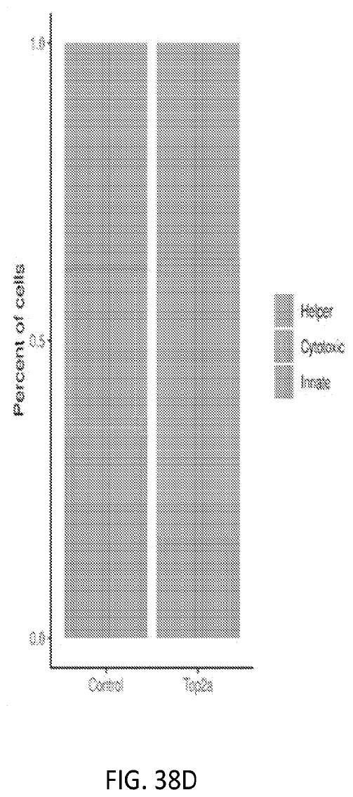

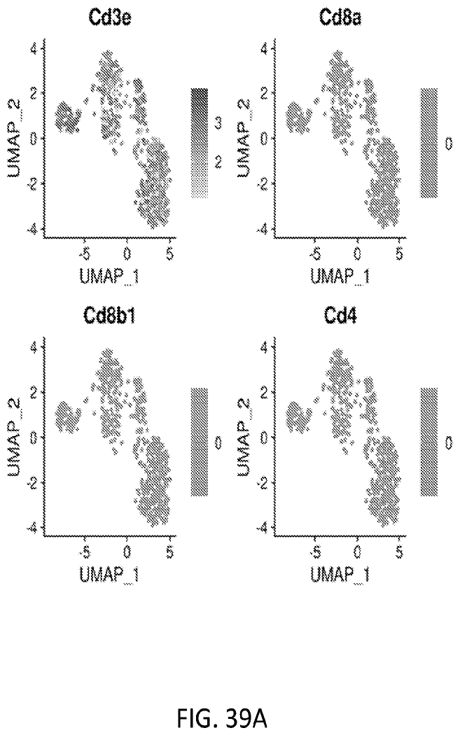

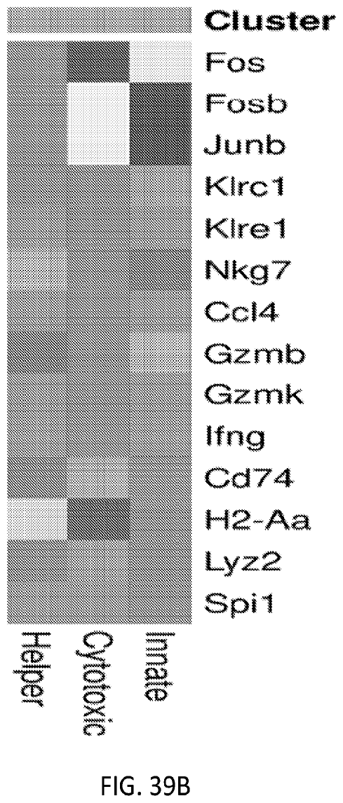

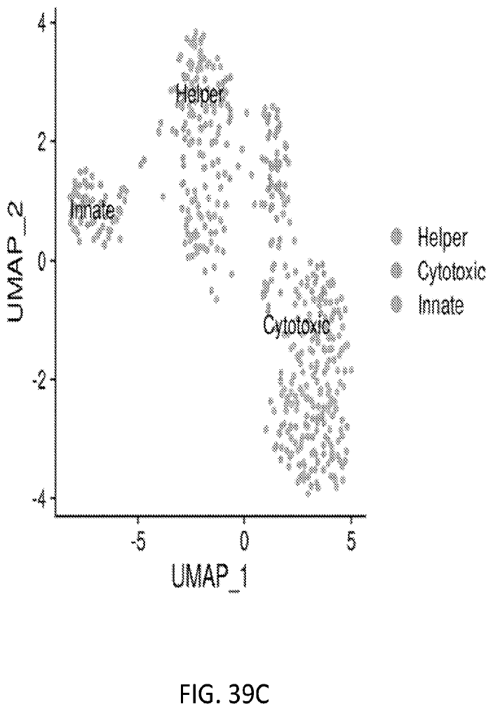

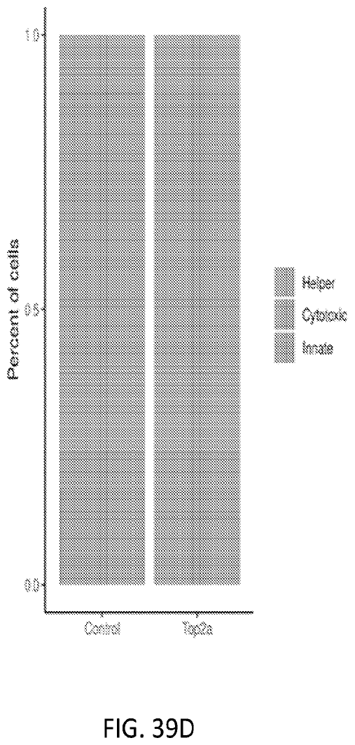

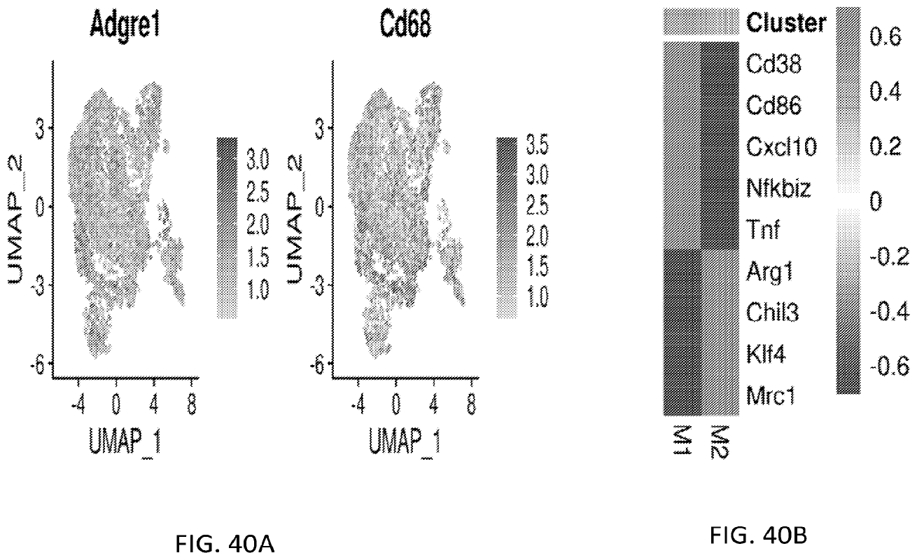

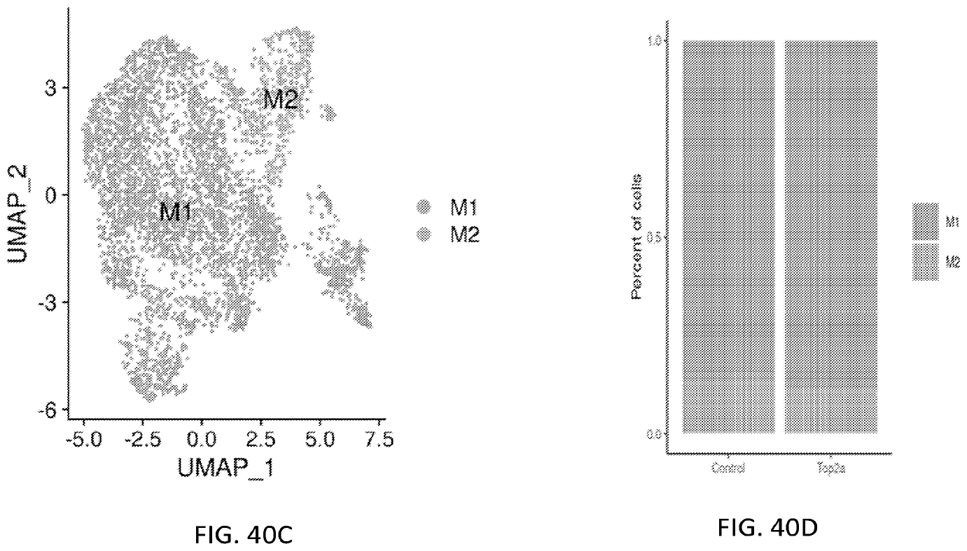

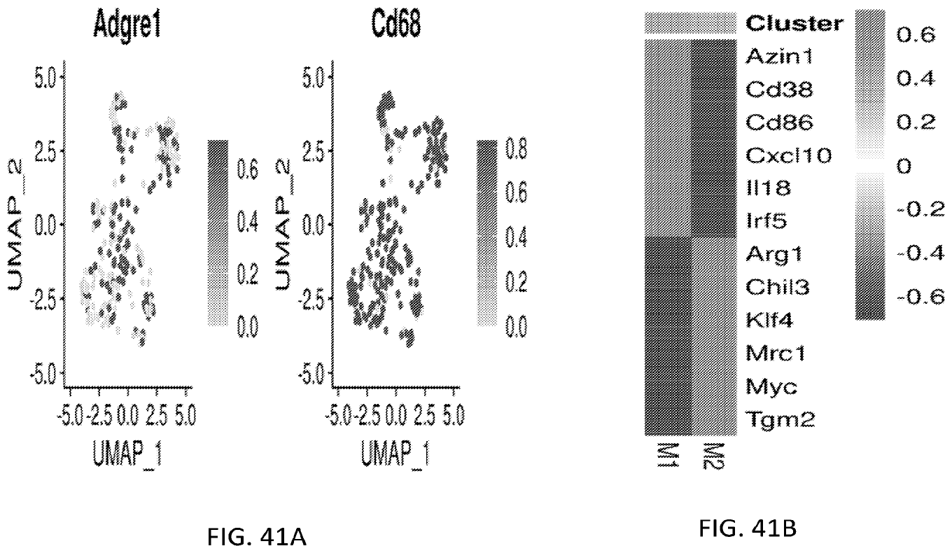

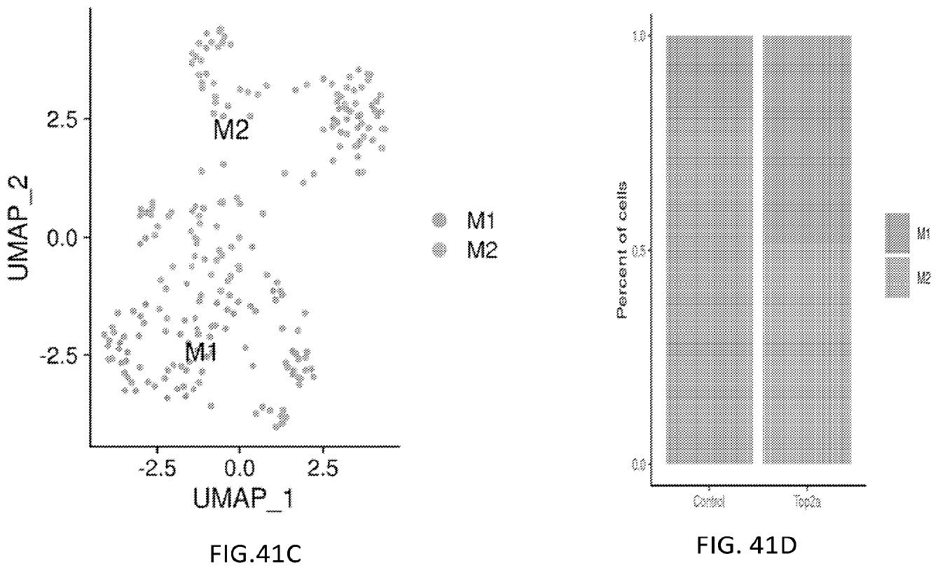

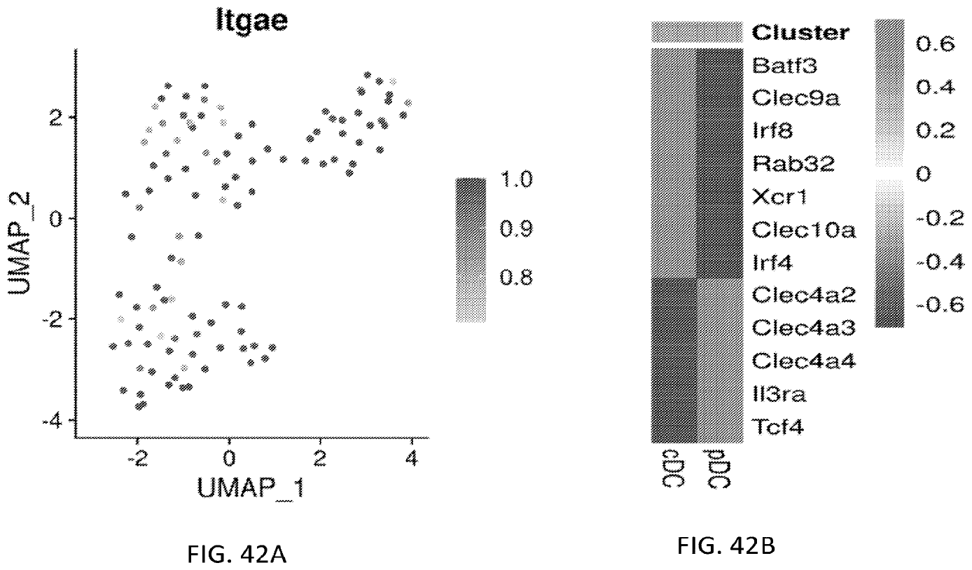

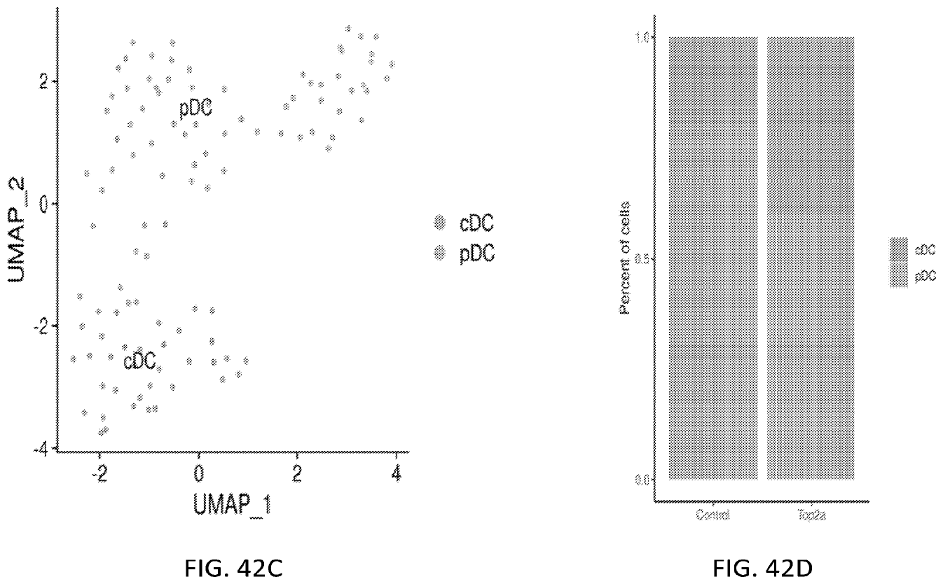

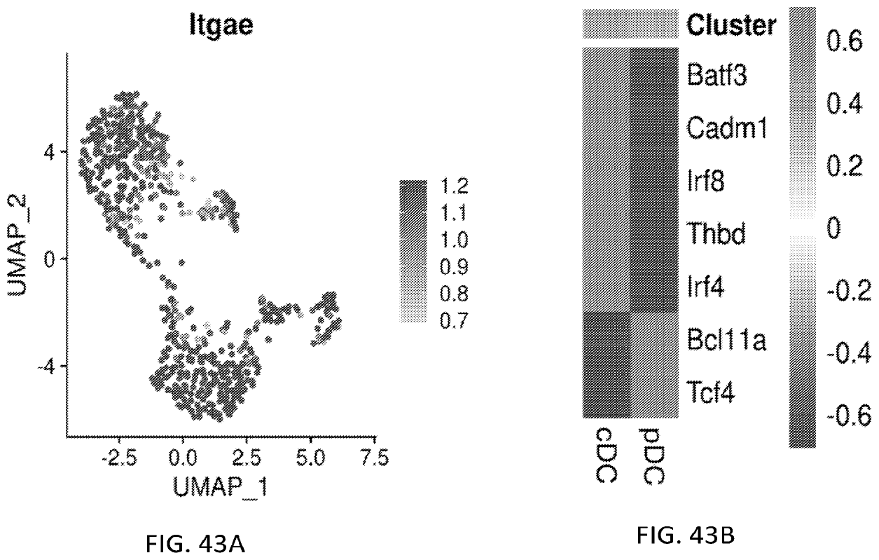

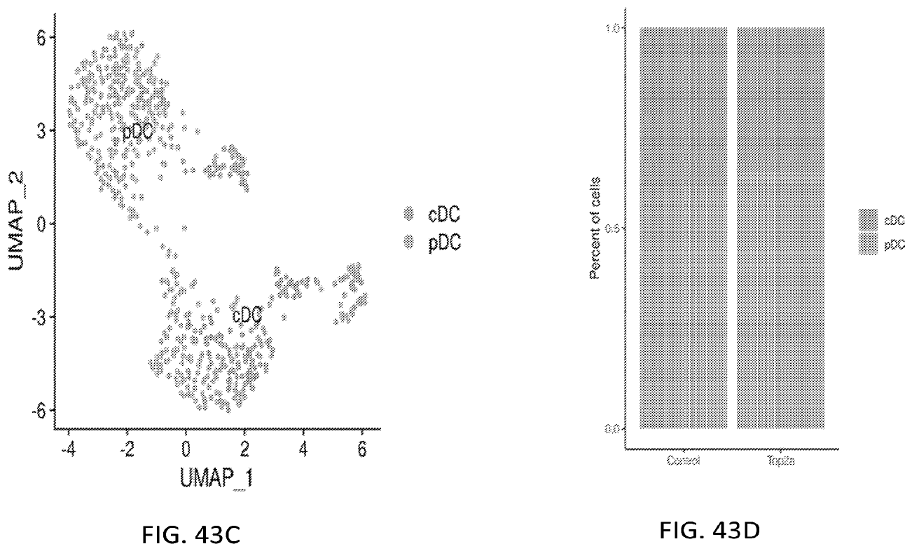

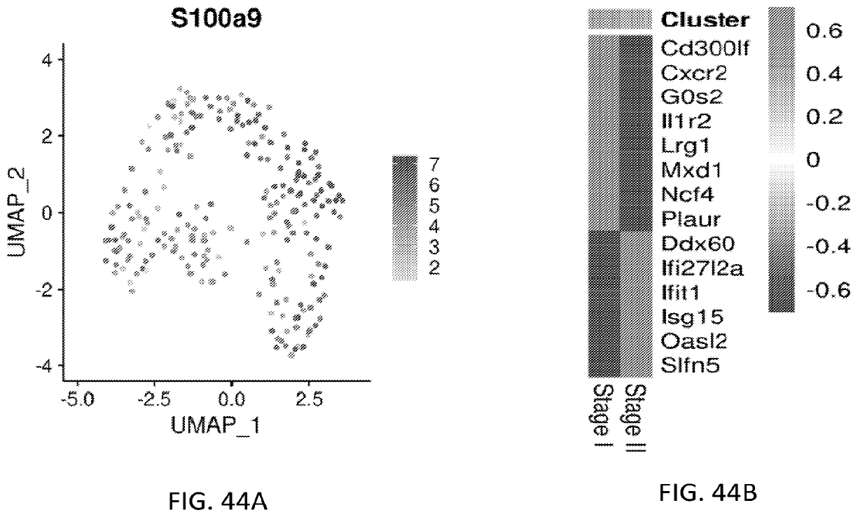

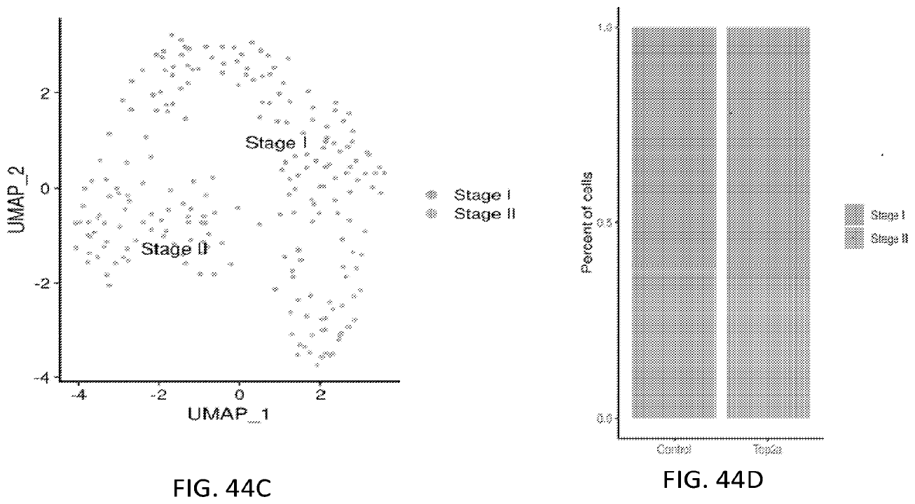

We examined tumor and lymph node tissues for changes in other immune cell populations after TOP2A vaccine treatment. For the DNT cells (CD4/CD8 double-negative T cells) in mouse breast tumor and lymph node tissues, the cells were divided into three subsets: helper, cytotoxic, and innate DNT cells (FIGS. 38A-38D and 39A-39D) 23. TOP2A vaccine treatment significantly increased the abundance of cytotoxic DNT cells, while the treatment decreased the proportion of innate DNT cells in mouse breast tumor tissues (FIG. 38D). The percentage of helper DNT cells was not altered by TOP2A vaccination (FIG. 38D). For the lymph node samples, TOP2A vaccine treatment increased the percentage of helper DNT cells but did not change the percentage of cytotoxic DNT cells (FIG. 39D). These results suggest that the TOP2A vaccine treatment specifically increases the abundance of cytotoxic DNT cells in mouse breast tumors. Macrophages were subdivided into M1 and M2 subsets according to their marker gene expression in both breast tumor and lymph node tissues (FIGS. 40A-40D and 41A-41D). The abundance of anti-tumor M1 macrophages was increased and the pro-tumor M2 macrophages decreased after TOP2A vaccination in both breast tumor and lymph node tissues (FIGS. 40D and 41D). These data suggest that macrophages also participate in the anti-tumor response induced by TOP2A vaccination. Two subsets of DC were detected in tissues of treated mice: conventional DC (CDC) and plasmacytoid DC (pDC) based on the analysis of corresponding marker genes (FIGS. 42A-42D and 43A-43D) 24. TOP2A vaccine treatment significantly increased the proportion of cDC in breast tumor tissues but not in lymph nodes. cDC are known to play a critical role in anti-tumor immunity²⁵. Finally, we detected neutrophils in the breast tumor samples. Neutrophils can be divided into Stage I and Stage II subsets²⁶, which represent progenitor and mature neutrophils, respectively. We identified both neutrophil subsets in breast tumor tissues and found that TOP2A vaccine treatment did not change the proportions of these two subsets (FIGS. 44A-44C).

TCR clonotype analysis revealed the appearance of new T cell clones as a consequence of TOP2A vaccination.

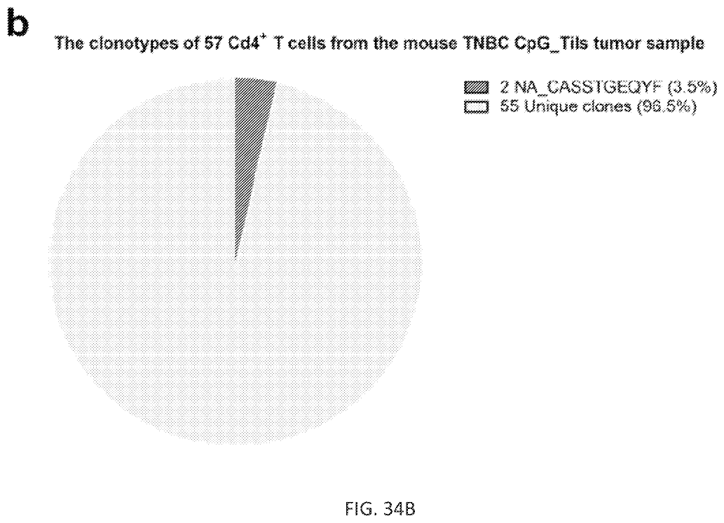

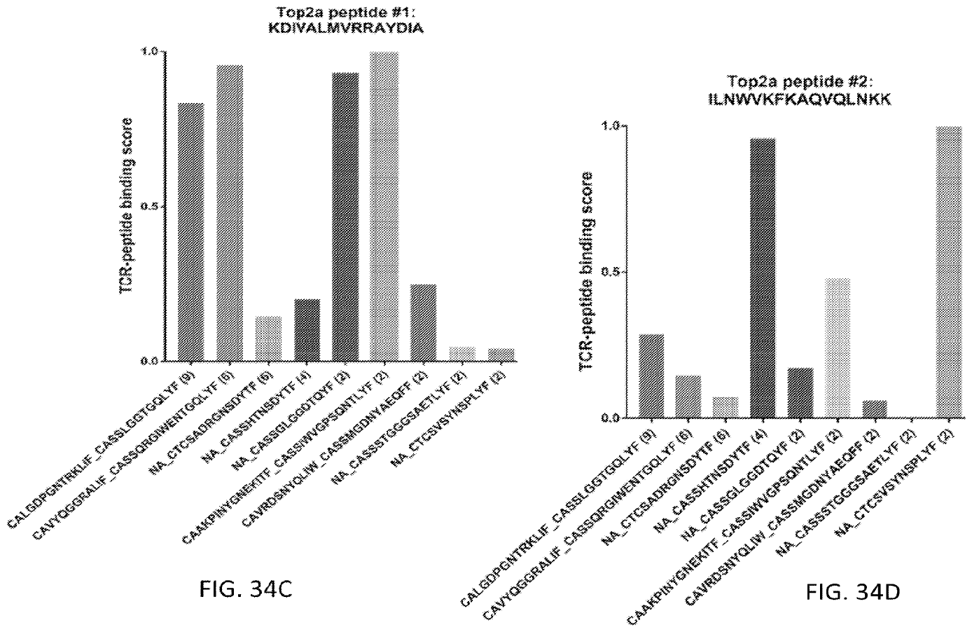

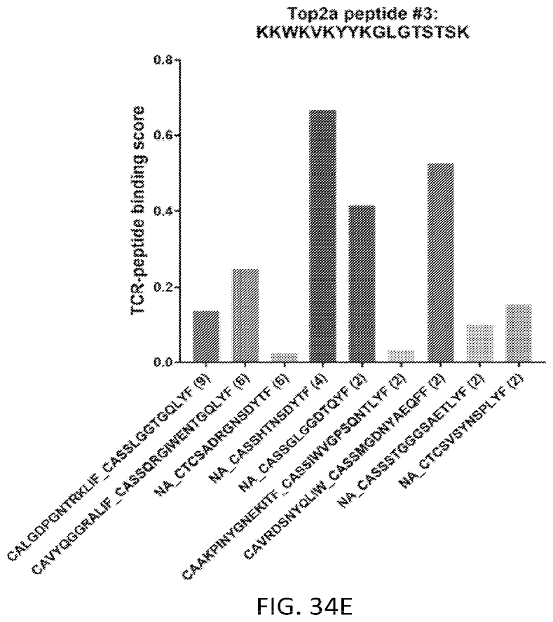

To explore potential mechanisms underlying the anti-tumor response induced by TOP2A vaccine treatment, we analyzed scTCRseq data of T cells from the control and TOP2A vaccine groups. We sequenced 201 CD4+ T cells in tumor tissues from TOP2A vaccinated mice and 57 CD4+ T cells in tumor tissues from the CpG control mice. The top five most frequent TCR clonotypes (frequency >=4) constituted 15.5% of the total CD4+ TCR clonotypes in TOP2A vaccine group; these clonotypes were not found in CpG control group, suggesting their involvement in the TOP2A vaccine generated CD4+ T cell immune response (FIG. 34A). Nearly all of the TCR clonotypes in vaccinated mice were unique versus the CpG controls (FIG. 34A-34E). To predict the likelihood of TCR-peptide binding for the TOP2A vaccine peptides, we used ERGO (pEptide tcR matchinG prediction) software which is a highly specific and generic TCR-peptide binding predictor (https://github.com/louzounlab/ERGO) 27. For the first TOP2A peptide-KDIVALMVRRAYDIA (SEQ ID NO: 1), the top 4 TCR clonotypes with the highest binding scores include: CAAKPINYGNEKITF_CASSIWVGPSQNTLYF (2 cells, clonotype frequency: 1%), CAVYQGGRALIF_CASSQRGIWENTGQLYF (6 cells, clonotype frequency: 3%), NA_CASSGLGGDTQYF (2 cells, clonotype frequency: 1%), and CALGDPGNTRKLIF_CASSLGGTGQLYF (9 cells, clonotype frequency: 4.5%) (FIG. 34C). For the second TOP2A peptide-ILNWVKFKAQVQLNKK (SEQ IS NO: 2), the top 4 clonotypes with the highest binding scores were: NA_CTCSVSYNSPLYF (2 cells, clonotype frequency: 1%), NA_CASSHTNSDYTF (4 cells, clonotype frequency: 2%), CAAKPINYG-NEKITF_CASSIWVGPSQNTLYF (2 cells, clonotype frequency: 1%), and CALGDPGNTRKLIF_CASSLGGTGQLYF (9 cells, clonotype frequency: 4.5%) (FIG. 34D). For the third TOP2A peptide-KKWKVKYYKGLGTSTSK (SEQ ID NO: 3), the top 4 clonotypes with the highest binding scores are: NA_CASSHTNSDYTF (4 cells, clonotype frequency: 2%), CAVRDSNYQ-LIW_CASSMGDNYAEQFF (2 cells, clonotype frequency: 1%), NA_CASSGLGGDTQYF (2 cells, clonotype frequency: 1%), and CAVYQGGRALIF_CASSQRGIWENTGQLYF (6 cells, clonotype fre-quency: 3%) (FIG. 34E). Notably, the top two most frequent TCR clonotypes, CALGDPGNTRKLIF_CASSLGGTGQLYF and CAVYQGGRALIF_CASSQRGIWENTGQLYF, both showed high binding affinity to the TOP2A peptide KDIVALMURRAYDIA (SEQ ID NO: 1) (FIG. 34C), suggesting that this TOP2A peptide plays an important role in the TOP2A vaccine-induced anti-tumor T cell response.

### Discussion

In the current study, we developed a multi-peptide vaccine targeting TOP2A and showed that this multi-peptide TOP2A vaccine was strikingly effective in preventive efficacy studies with C3 (1)/Tag transgenic mice. Top2A vaccination increased CD4+ and CD8+ tumor infiltrating lymphocytes as well as the percentage of the functionally activated CD4+ and CD8+ cells in the spleens from the vaccinated mice. TOP2A vaccination showed no toxicity when all key organs from the vaccinated C3 (1)/Tag mice, suggesting that the TOP2A vaccine is generally safe and does not induce overt autoimmunity. In addition, we showed that a long-term memory response occurs in TOP2A-vaccinated C3 (1)/Tag mice by their rejection of a secondary challenge with M6 mammary carcinoma cells.

TOP2A is an enzyme that controls the topological state of DNA. It catalyzes double-stranded DNA breaks and promotes gene transcription during mitosis²⁸, and is overexpressed in several malignancies including breast cancer, ovarian cancer and prostate cancer. TOP2A is directly associated with tumor cell proliferation and invasiveness in breast cancer, and expression has been reported to be amplified in TNBC patients with high-risk factors such as large tumor size, high-grade tumor, and lymph node infiltration 14,29,30 Here. we identified overexpression of the TOP2A gene in both mouse breast tumor cell lines and human TNBC cancer samples (FIGS. 35A-35F). We also confirmed the expression of TOP2A in TNBCs from AA women. The incidence of TNBC has been reported to be higher in AA women and is associated with a poorer prognosis as compared to European American women, presumably due to various socio ors in addition to the increased likelihood of developing 31,52

The potential usefulness of any specific animal model of cancer is whether it reasonably parallels the comparable human disease. This is easier to achieve if the cancer is driven by a clearly defined mutation or amplification e.g. pancreatic cancer (KRAS mutation), colon cancers (mutations in APC or WNT pathway), squamous cell skin cancer (p53 mutations), HER2 positive breast cancer (amplification of HER2). In contrast, sporadic TNBC in humans does not have a single driving mutation although the preponderance of tumors has p53 mutations and a loss of RB^{15,16}. Tumors derived from the C3 (1)/Tag mouse model similarly have loss of function of p53 and RB. Breast Cancer in the C3 (1)/Tag mouse model develops through atypical ductal hyperplasia and DCIS starting around 10 weeks of age and progress to mammary adenocarcinomas after 12 weeks of age³³. Most importantly, tumors from this model appear similar to human TNBC when compared by RNA expression and genomic alterations (amplifications and deletions) 15,16. Thus, the model appears to be a relatively good model of human TNBC and responds to certain cytotoxic agents which have proven useful against human TNBC. In the present study, TOP2A vaccination was started before cancer development in C3 (1)/Tag mice (FIG. 30D), and the vaccine effectively inhibited the tumor volume as compared with the adjuvant control (FIG. 30E). Interestingly tumor incidence was markedly different between the adjuvant control and TOP2A vaccine groups. In TOP2A vaccinated mice, tumors developed in only 27% of mice, whereas all adjuvant-treated controls developed cancer. We observed no TOP2A vaccine-associated toxicities on major organs including the brain, kidney, liver, lung, and bone marrow (data was not shown). Similarly, no toxicities were observed after vaccinating mice with vaccines targeting EGFR, HER2, or IGFBP-2 peptides in humans^{9,10,34,35}.

Peptides from any protein expressed within a given cell can be intracellularly processed in the ER and TAP and eventually presented on the cell surface but are normally ignored by the immune system since most of those T cells have been deleted from the T cell repertoire. However, in the case of over-expressed self-proteins, these have been known to drive T cell responses from small percentages of “low” affinity (cryptic) T cell epitopes present in the repertoire. Furthermore, as some of the tumor cells die, the overexpressed proteins can be taken up by APCs and presented to both CD4 & CD8 T cells. CD4 T cells can differentiate into various Th subsets that can induce, modulate and maintain immune responses to tumor antigens. These CD4 T cell subsets include Th1, Th2, and regulatory T cells (Tregs). The Th1 subset produces IFN-γ, TNF-α and IL-2, which regulate cellular immunity and play important roles in anti-tumor immune responses¹⁰. The TOP2A vaccine employed in the current study induced predomi-nantly Th1 cell responses, without eliciting a strong Th2 response. Type I cytokines secreted by Th1 cells, such as IFN-γ, can up-regulate MHC class I expression on the membrane of tumor cells as well as APC, which can facilitate tumor recognition by CD8+ T cells³⁶. Moreover, Th1 cells can also facilitate the cross-presentation of MHC class I binding peptide antigens to CD8+ T cells^{10,37}. These mechanisms could be involved in generating the Th1 responses associated with the TOP2A vaccine, and ultimately facilitating the generation of the CD8+ T cell response. It is also possible that Top2A MHC II peptides could also induce immune responses for CD8+ T cells via cross-priming in the vaccinated mice. Furthermore, our MHC Class-II peptides contain two Class-I peptide epitopes: P235 (VALMURRAY) (SEQ ID NO: 32) within Class-II peptide P232 and P414 (VKFKAQVQL) (SEQ ID NO: 33) within Class-II peptide P410. Cytotoxic CD8+ T cells play an important role in the anti-tumor immune response by direct killing of tumor cells³⁸ Our study demonstrated that the TOP2A vaccine could stimulate both local and systemic antigen specific CD4+ and CD8+ T cell responses. We also found a significant increase in IL-23 secretion from splenocytes isolated from TOP2A vaccinated mice. IL-23 is important and necessary for the differentiation of Th17 lymphocytes³⁹. This is notable as IL-23 is known to play a role in antitumor activity^{40,41}.

In summary, we showed here that a TOP2A multi-peptide-based vaccine has preventive activity in a TNBC mouse model. TOP2A vaccination induced a Th1 immune response that significantly decreased TNBC tumor formation in both syngeneic and GEM models. Meanwhile, no toxicity was observed in vaccinated mice. We designed 15-17 amino acid peptides with 100% sequence identity between human and mouse TOP2A, making it possible that these peptides could be directly translated into clinical studies. Overall, our data demonstrate that the multi-peptide TOP2A vaccine is highly immunogenic and has the potential to be efficacious in the immunoprevention of TNBC in humans.

### Methods

### Mice

Transgenic C3 (1)/Tag mice and C3 (1)/Tag-REAR (abbreviation for rearrangement) mice were a generous gift from Dr. Jeffery E. Green¹⁷. FVB/N wild-type mice were bought from the Jackson Laboratory. For all experiments, only F2 C3 (1)/Tag or C3 (1)/Tag-REAR generation mice were used. Mice were acclimatized one week following arrival to the facility. All mice were euthanized through CO2 inhalation in the mouse cage and followed by cervical dislocation when experiments reached at end point. Mice were maintained and bred in the Biomedical Resource Center at the Medical College of Wisconsin (MCW), Milwaukee, WI and the Houston Methodist Research Institute, Houston, TX. All procedures were approved by the Institutional Animal Care and Use Committee (IACUC).

### Cell Lines

M27 (weakly tumorigenic/benign tumor), M6 (malignant tumor), and M6C (metastatic tumor) and M28 (normal control) cells, all derived from C3 (1)/Tag mice, were provided by Dr. Jeffery E. Green⁴². The cell lines were maintained in DMEM medium containing high glucose (Gibco), supplemented with 5% fetal bovine serum, penicillin/streptomycin and sodium pyruvate (Invitrogen).

### Immunohistochemistry (IHC)

IHC staining was performed by the Children's Research Institute Histology Core at MCW. Leica Bond Immunostainer Max (model #10897664) and Leica Bond Immunostainer RX system (model #11784892) were used for human tissue microarray (TMA) and murine samples, respectively. Mouse mammary gland samples from C3 (1)/Tag or C3 (1)/Tag-REAR mice were formalin-fixed and paraffin-embedded (Sakura Tissue Tek VIP5). Four 4 μm sections were used for IHC staining. The numbers of CD4+ and CD8+ tumor-infiltrating T-lymphocytes (TIL) were determined as cells per mm2 tumor area by using CD4 antibody (Invitrogen 14-9766-82) and CD8 antibody (Invitrogen 14-0808-82).

### ELISpot Assay

Cell suspensions from whole spleens were filtered through a 70 μm cell strainer (BD) and subjected to red blood cell lysis using ACK lysis buffer. 3.0×10⁵ cells were plated into individual wells of a MAIPS4510 multiscreen 96-well plate coated with anti-interferon γ (IFN-γ) detection antibody and containing media with either peptide, concanavalin A (positive control), HIV peptide (negative control), or no antigen (negative control). After 72-hour incubation, plates were washed and incubated with a secondary antibody (BD) overnight at 4° C. Wells were then washed with PBS and HRP streptavidin was added. Following one-hour incubation, the plate was developed using AEC substrate for between five to 25 minutes. An automated plate reader system (CTL Technologies) was used to image the plates and quantify spot numbers.

### Scoring System for the Prediction of MHC Class II Binding Epitopes

A combined scoring system published by Dr. Disis and colleagues was used to identify selected antigen-specific MHC epitopes with optimal binding affinity³⁴. Briefly, to identify antigen-specific MHC class II epitopes with optimal binding affinity and promiscuity across multiple alleles, the following algorithms were used for prediction: NetMHCIIpan (https://services.healthtech.dtu.dk/service.php?NetMHCIIpan-4.0, Technical University of Denmark, Lyngby, Denmark) and Rankpep (http://imed.med.ucm.es/Tools/rankpep.html, University Computense Madrid, Harvard, Madrid, Spain). For each available MHC class II allele, 20 peptide sequences were initially selected solely based on the rank-order of the predicted binding affinity from each algorithm. The sequences are approximately 15 amino acids in length. Individual amino acids for each selected peptide were assigned a score, with one being an amino acid contained in a peptide sequence that ranked highest for predictive binding affinity. Scoring individual amino acids accounted for the multiple-peptides overlaps occurring within and across algorithms. The scores(S) for each amino acid were summed up across the multiple MHC class II alleles from two algorithms. Then, the number (N) of MHC class II alleles, for which each amino acid was predicted to have high-affinity binding, was counted. The final score for each amino acid was calculated by multiplying S and N. For ease of identifying the most potentially immunogenic segments of the protein, each amino acid was assigned a color (from dark red to light blue) based on its final score percentile, with dark red being highest at ≥75% and light blue the lowest at <10%. The color strata are as follows: dark red ≥75% of highest score; red=50~75% of highest score; orange=40~50% of highest score; yellow=30~40% of highest score; green=20~30% of highest score; blue≤20% of highest score. Thus, the dark red color corresponds to a sequence where multiple peptides scored highly within an algorithm as well as across algorithms. Light blue represents sequences that are the least potentially immunogenic of all predicted high-binding peptides.

### Vaccine Preparation and Immunization

A total of 150 μg of vaccine, including 50 μg of each individual peptide, was administered to the mice. Three different TOP2A peptides were purchased from Genemed Synthesis and diluted in phosphate-buffered saline (PBS) to 50 μl/mouse. Peptides and equal amounts of adjuvant CpG (Class B CpG oligonucleotide; a murine TLR9 ligand, Cat. No. tlrl-1826, InvivoGen) were added to bring the total vaccine volume to 100 μl/mouse. CpG was used at 50 μg per mouse. Mice were injected subcutaneously with TOP2A vaccines following the timelines shown in FIGS. 30D, 31A, 32A.

### In Vivo Tumorigenicity Assay

In the syngeneic model, C3 (1)/Tag-REAR mice were generated from a C3 (1)/Tag founder line through the loss of one of the original multiple copies of the C3 (1)/Tag-antigen transgene resulting in no spontaneous cancer phenotype¹⁷. M6 cells, derived from a C3 (1)/Tag transgenic mammary tumor, were implanted into the mammary fat pad of C3 (1)/Tag-REAR mice. M6 cells were washed, resuspended in PBS at a density of 1×10⁶ cells in 100 μl PBS, and injected into the #4 mammary fat pads of female C3 (1)/Tag-REAR mice. Following implantation, tumor diameters were measured using calipers and tumor volumes were calculated using the formula: maximum diameter×(minimum diameter)²×0.4. In the spontaneous GEM model, C3 (1)/Tag mice were treated with the TOP2A peptide vaccine following the experimental design in FIG. 30D. C3 (1)/Tag mice were euthanized at 20-weeks of age for estimation of tumor development. Tumor volumes were measured using calipers and calculated using the formula: maximum diameter×(minimum diameter)²×0.4.

### Cytokine Analysis

Mouse Th1/Th2/Th17 Cytokines Multi-AnalyteELISArray™ Kits (Qiagen) were used for cytokine analysis. The cytokines repre-sented by this array are IL2, IL4, IL5, IL6, IL10, IL12, IL13, IL17A, IL23, IFN-γ, TNFα, and TGFβ1. Splenocytes from different groups of mice were stimulated with different peptides for 72 hours, and then culture supernatants were collected and assayed based on the manufacturer's instructions.

### Flow Cytometry

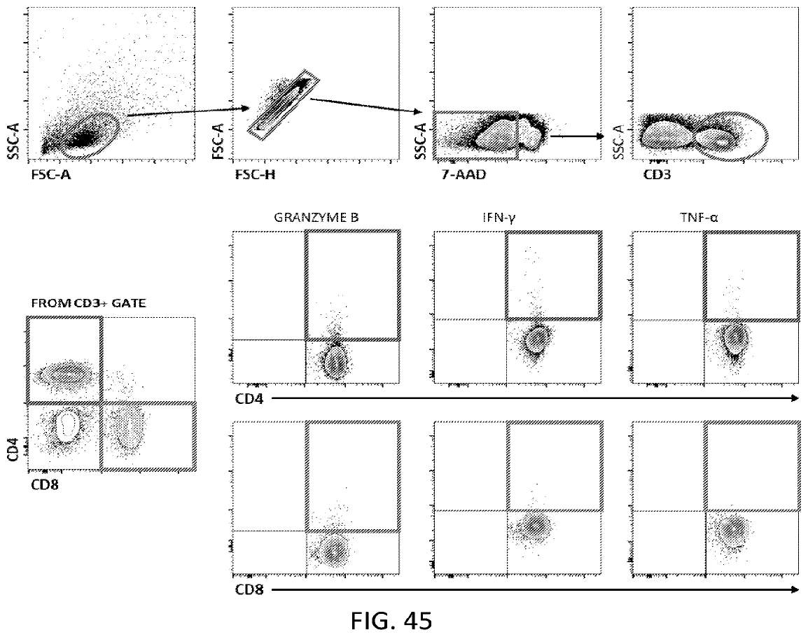

Cell pellets were incubated with surface markers of interest at the recommended or titrated concentrations, incubated at 4° C. for 30 minutes, and protected from light. After incubation, cells were washed and resuspended in FACS fixation buffer for either analysis or intracellular staining. To begin intracellular staining, cells were fixed with Foxp3/Transcription factor staining buffer set (eBioscience) and stained with intracellular markers of interest at the recommended or titrated concentrations at 4° C. for at least 30 minutes while protecting them from light. Samples were washed with permeabilization buffer and resuspended in FACS fixation buffer. Stained cells were fixed in 1% paraformaldehyde and permeabilized following the manufacturer's instructions to evaluate the expression of intracellular targets, granzyme B, IFN-γ and TNF-α. Flow cytometry was conducted using an LSR-II flow cytometer (BD). Data were analyzed using FlowJo software (Tree Star). FACS sequential gating/sorting strategies are provided in FIG. 45.

Single-Cell RNA Sequencing (scRNAseq) and TCR Sequencing (scTCRseq)

For scRNAseq, randomly selected lymph node samples (1 sample from CpG control group and 2 samples from TOP2A vaccine treated group) and the breast tumor samples (1 sample from CpG control group and 1 sample from TOP2A vaccine treated group) of C3 (1)/Tag mice were harvested at the end of the study, minced and digested at 37° C. for 30 minutes with mouse tumor dissociation buffer (Miltenyi Biotec, CA) to generate single-cell suspensions per the manufacturer's instructions. The processed samples were directly stained with violet viability dye, APC anti-CD45, and CD45+ leukocytes were sorted using FACS. FACS-sorted CD45+ leukocytes were then spun down at 300 g for 5 minutes and counted manually with a Neubauer Chamber. Approximately 2.0×10⁴ cells were loaded onto the 10× Chromium Controller per the manufacturer's instruction, resulting in a recovery of about 1×10⁴ cells. For the lymph node samples, the libraries of single-cell transcriptome were generated by Chromium Single Cell 3′ v3 Reagent Kits (10× Genomics). For tumor samples, single-cell transcriptome and single-cell TCR libraries were prepared using a 10× Chromium Single-cell 5′ and VDJ library construction kit. All of the libraries were sequenced using NextSeq 500/550 High Output Kits v2 (150 cycles) (Illumina) according to the manufacturer's protocol.

scRNA-Seq Data Analysis

Raw sequencing data were de-multiplexed and converted to gene-barcode matrices using the Cell Ranger (version 2.2.0) mkfastq and count functions, respectively (10×Genomics). The mouse reference genome mm10 was used for alignment. Data were further analyzed in R (version 3.4.0) using Seurat (version 3). The number of genes detected per cell, number of unique molecular identifiers (UMIs), and the percent mitochondrial genes were plotted, and outliers were removed to filter out doublets and dead cells. Raw UMI counts were normalized and log transformed. Integrated analysis was then performed to identify shared cell clusters that are present across different datasets. Principal component analysis was performed using variable genes, and the top 20 most statistically significant principal components were used for UMAP analysis.

### Statistical Analysis

All in vitro assays were performed at least in triplicate. Six to twelve mice per group were used for the in vivo studies. A two-tailed Student's t-test was used to evaluate differences between the control group and each treatment group. P-values<0.05 were considered statistically significant.

### REFERENCES FOR EXAMPLES 1-14

Aprelikova O, Tomlinson C C, Hoenerhoff M, Hixon J A, Durum S K, Qiu T H, He S, Burkett S, Liu Z Y, Swanson S M, Green J E. Development and Preclinical Application of an Immunocompetent Transplant Model of Basal Breast Cancer with Lung, Liver and Brain Metastases. PloS One. 2016 May 12; 11 (5). PMID: 27171183
Bachelier R, Xu X, Li C, Qiao W, Furth P A, Lubet R A, Deng C X. Effect of bilateral oophorectomy on mammary tumor formation in BRCA1 mutant mice. Oncol Rep. 2005 November; 14 (5): 1117-20. PMID: 16211273.
Becht E, McInnes L, Healy J, Dutertre C A, Kwok I W H, Ng L G, Ginhoux F, Newell E W. Dimensionality reduction for visualizing single-cell data using UMAP. Nat Biotechnol. 2018. Epub 2018 Dec. 12. Doi: 10.1038/nbt.4314. PubMed PMID: 30531897.
Butler A, Hoffman P, Smibert P, Papalexi E, Satija R. Integrating single-cell transcriptomic data across different conditions, technologies, and species. Nat Biotechnol. 2018; 36 (5): 411-20. Epub 2018 Apr. 3. PubMed PMID: 29608179.
Carmona S J, Siddiqui I, Bilous M, Held W, Gfeller D. Deciphering the transcriptomic landscape of tumor-infiltrating CD8 lymphocytes in B16 melanoma tumors with single-cell RNA-Seq. Oncoimmunology. 2020; 9 (1): 1737369. Epub 2020 Apr. 22. Doi: 10.1080/2162402X.2020.1737369. PubMed PMID: 32313720; PMCID: PMC7153840.
Carey L. A., Perou C. M., Livasy C. A., Dressler L. G., Cowan D., Conway K., Karaca G., Troester M. A., Tse C. K., Edmiston S., et al. Race, breast cancer subtypes, and survival in the Carolina Breast Cancer Study. JAMA. 2006 Jun. 7; 295 (21): 2492-502. PMID: 16757721
Cecil, D. L., et al., Elimination of IL-10-inducing T-helper epitopes from an IGFBP-2 vaccine ensures potent antitumor activity. Cancer Res, 2014. 74 (10): p. 2710-8
Disis, M. L., et al., Peptide-based, but not whole protein, vaccines elicit immunity to HER-2/neu, oncogenic self-protein. J Immunol, 1996. 156 (9): p. 3151-8.
Disis M L, Gad E, Herendeen D R, Lai V P, Park K H, Cecil D L, O'Meara M M, Treuting P M, Lubet R A. A multiantigen vaccine targeting neu, IGFBP-2, and IGF-IR prevents tumor progression in mice with preinvasive breast disease. Cancer Prev Res (Phila). 2013; 6:1273-1282.
Ebben J D, Lubet R A, Gad E, Disis M L, You M. Epidermal growth factor receptor derived peptide vaccination to prevent lung adenocarcinoma formation: An in vivo study in a murine model of EGFR mutant lung cancer. Mol Carcinog. 2016 November; 55 (11): 1517-1525.
Gao J J, Swan S M. Luminal A Breast Cancer and Molecular Assays: A Review. Oncologist. 2018 May; 23 (5): 556-565.
Giladi A, Paul F, Herzog Y, Lubling Y, Weiner A, Yofe I, Jaitin D, Cabezas-Wallscheid N, Dress R, Ginhoux F, Trumpp A, Tanay A, Amit I. Single-cell characterization of haematopoietic progenitors and their trajectories in
homeostasis and perturbed haematopoiesis. Nature Cell Biology. 2018; 20 (7): 836-46. Doi: 10.1038/s41556-018-0121-4.
Green J E, Shibata M A, Yoshidome K, Liu M L, Jorcyk C, Anver M R, Wigginton J, Wiltrout R, Shibata E, Kaczmarczyk S, Wang W, Liu Z Y, Calvo A, Couldrey C, The C3 (1)/SV40 T-antigen transgenic mouse model of mammary cancer: ductal epithelial cell targeting with multistage progression to carcinoma. Oncogene. 2000 Feb. 21; 19 (8): 1020-7. PMID: 10713685
Harbeck N, Gnant M, Breast cancer, The Lancet, Volume 389, Issue 10074, 18-24 Mar. 2017, Pages 1134-1150
Herschkowitz J I, Simin K, Weigman V J, Mikaelian I, Usary J, Hu Z, Rasmussen K E, Jones L P, Assefnia S, Chandrasekharan S, Backlund M G, Yin Y, Khramtsov A I, Bastein R, Quackenbush J, Glazer R I, Brown P H, Green J E, Kopelovich L, Furth P A, Palazzo J P, Olopade O I, Bernard P S, Churchill G A, Van Dyke T, Perou C M. Identification of conserved gene expression features between murine mammary carcinoma models and human breast tumors. Genome Biol. 2007; 8 (5): R76. PMID: 17493263.
Holzer R G, MacDougall C, Cortright G, Atwood K, Green J E, Jorcyk C L. Development and characterization of a progressive series of mammary adenocarcinoma cell lines derived from the C3 (1)/SV40 Large T-antigen transgenic mouse model. Breast Cancer Res Treat. 2003
Jemal A, Bray F, Center M M, Ferlay J, Ward E, Forman D. Global cancer statistics CA A Cancer J. Clin., 61 (2) (2011), pp. 69-90
Jones L P, Li M, Halama E D, Ma Y, Lubet R, Grubbs C J, Deng C X, Rosen E M, Furth P A. Promotion of mammary cancer development by tamoxifen in a mouse model of Brca1-mutation-related breast cancer. Oncogene. 2005 May 19; 24 (22): 3554-62. PMID: 15750629.
Klintman M, Buus R, Cheang M C U, Sheri A, Smith I E, Dowsett M, Changes in Expression of Genes Representing Key Biologic Processes After Neoadjuvant Chemotherapy in Breast Cancer, and Prognostic Implications in Residual Disease. Clin Cancer Res. 2016 May 15; 22 (10): 2405-16. PMID: 27179111
Li R, Zheng C, Wamg Q, Bi E, Yang M, Hou J, Fu W, Yi Qi, Qian J, Identification of an immunogenic DKK1 long peptide for immunotherapy of human multiple myeloma. Haematologica. 2021 Mar. 1; 106 (3): 838-846. PMID: 32079700
Lo C H, Lee S C, Wu P Y, Pan W Y, Su J, Cheng C W, Roffler S R, Chiang B L, Lee C N, Cheng-Wu W, Tao M H, Antitumor and Antimetastatic Activity of IL-23. J Immunol. 2003 Jul. 15; 171 (2): 600-7. PMID: 12847224
Lollini P L, Cavallo F, Nanni P, Forni G. Vaccines for tumour prevention. Nat Rev Cancer 2006; 6 (3): 204-16. PMID: 16498443
Lowenfeld L, Mick R, Datta J, Xu S, Fitzpatrick E, Fisher C S, Fox K R, DeMichele A, Zhang P, Weinstein S, Roses R E, Czerniecki B J. Dendritic Cell Vaccination Enhances Immune Responses and Induces Regression of
HER2pos DCIS Independent of Route: Results of Randomized Selection Design Trial. Clin Cancer Res. 2016 Dec. 13. Pii: clincanres. 1924.2016. [Epub ahead of print] PMID: 27965306.
Ma X, Shou P, Smith C, Chen Y, Du H, Sun C, Kren N P, Michaud D, Ahn S, Vincent B, Savoldo B, Pylayeva-Gupta Y, Zhang S, Dotti G, Xu Y, Interleukin-23 Engineering Improves CAR T Cell Function in Solid Tumors. Nat Biotechnol. 2020 April; 38 (4): 448-459. PMID: 32015548
Martinez-Lostao L, Anel, A. & Pardo, J. How do cytotoxic lymphocytes kill cancer cells? Clin. Cancer Res. 21, 5047-5056 (2015).
Matsuo M, Nagata Y, Sato E, Atanackovic D, Valmori D, Chen Y T, et al. IFN-gamma enables cross-presentation of exogenous protein antigen in human Langerhans cells by potentiating maturation. Proceedings of the National Academy of Sciences of the United States of America. 2004; 101 (40): 14467-72
Mittendorf E A, Gurney J M, Storrer C E, Shriver C D, Ponniah S, Peoples G E. Vaccination with a HER2/neu peptide induces intra- and inter-antigenic epitope spreading in patients with early stage breast cancer. Surgery. 2006; 139:407-418.
Murphy T L, Murphy K M. Dendritic cells in cancer immunology. Cellular & Molecular Immunology. 2022; 19 (1): 3-13. Doi: 10.1038/s41423-021-00741-5.
Nakagawa M, Bando Y, Nagao T, Morimoto M, Takai C, Ohnishi T, Honda J, Moriy T, Izumi K, Takahashi M,
Sasa M, Tangoku A, Z Expression of p53, Ki-67, E-cadherin, N-cadherin and TOP2A in triple-negative breast cancer. Anticancer research. 2011; 31 (6): 2389-93.
Onitilo A A, Engel J M, Greenlee R T, Mukesh B N, Breast Cancer Subtypes Based on ER/PR and Her2 Expression: Comparison of Clinicopathologic Features and Survival. Clin Med Res. 2009 June; 7 (1-2): 4-13. PMID: 19574486
Park K H, Gad E, Goodell V, Dang Y, Wild T, Higgins D, Fintak P, Childs J, Dela Rosa C, Mary L Disis. Insulin-like growth factor-binding protein-2 is a target for the immunomodulation of breast cancer. Cancer Res 2008; 68:8400-9. PMID: 18922913
Pan J, Zhang Q, Sei S, Shoemaker R H, Lubet R A, Wang Y, You M. Immunoprevention of KRAS-driven lung adenocarcinoma by a multipeptide vaccine. Oncotarget. 2017 Aug. 1; 8 (47): 82689-82699. PMID: 29137294
Pei Y F, Yin X M, Liu X Q, TOP2A Induces Malignant Character of Pancreatic Cancer Through Activating β-Catenin Signaling Pathway. Biochim Biophys Acta Mol Basis Dis. 2018 January; 1864 (1): 197-207. PMID: 29045811
Pei Y F, Yin X M, Liu X Q, TOP2A Induces Malignant Character of Pancreatic Cancer Through Activating β-Catenin Signaling Pathway. Biochim Biophys Acta Mol Basis Dis. 2018 January; 1864 (1): 197-207. PMID: 29045811.
Prat A., Parker J S, Karginova O, Fan C, Livasy C, Herschkowitz J I, He X, Perou C M, Phenotypic and molecular characterization of the claudin-low intrinsic subtype of breast cancer. Breast Cancer Res, 2010. 12 (5): p. R68.
Schneble E J, Berry J S, Trappey F A, Clifton G T, Ponniah S, Mittendorf E, Peoples G E. The HER2 peptide nelipepimut-S(E75) vaccine (NeuVax™) in breast cancer patients at risk for recurrence: correlation of immunologic data with clinical response. Immunotherapy. 2014; 6 (5): 519-31. PMID: 24896623.
Siddharth S and Sharma D, Racial Disparity and Triple-Negative Breast Cancer in African American Women: A Multifaceted Affair between Obesity, Biology, and Socioeconomic Determinants. Cancers (Basel). 2018 December; 10 (12): 514. PMID: 30558195
Siegel R L, Miller K D, Fuchs H E, Jemal A, Cancer statistics, 2021. CA Cancer J. Clin. 2021, 41, 7-333. PMID: 33433946.
Springer I, Besser H, Tickotsky-Moskovitz N, Dvorkin S, Louzoun Y. Prediction of Specific TCR-Peptide Binding From Large Dictionaries of TCR-Peptide Pairs. Front Immunol. 2020; 11:1803. Epub 2020 Sep. 29. doi: 10.3389/fimmu.2020.01803. PubMed PMID: 32983088; PMCID: PMC7477042.
Stuart T, Butler A, Hoffman P, Hafemeister C, Papalexi E, Mauck W M, 3rd, Hao Y, Stoeckius M, Smibert P, Satija R. Comprehensive Integration of Single-Cell Data. Cell. 2019; 177 (7): 1888-902 e21. Epub 2019 Jun. 11. PubMed PMID: 31178118.
Villar J, Segura E. Decoding the Heterogeneity of Human Dendritic Cell Subsets. Trends Immunol. 2020; 41 (12): 1062-71. Epub 2020 Dec. 1. doi: 10.1016/j.it.2020.10.002. PubMed PMID: 33250080.
Wright M H, Robles A I, Herschkowitz J I, Hollingshead M G, Anver M R, Perou C M, Varticovski L. Molecular analysis reveals heterogeneity of mouse mammary tumors conditionally mutant for Brca1. Mol Cancer. 2008 Apr. 7; 7:29. doi: 10.1186/1476-4598-7-29. PMID: 18394172.
Yang L, Zhu Y, Tian D, Wang S, Guo J, Sun G, Jin H, Zhang C, Shi W, Gershwin M E, Zhang Z, Zhao Y, Zhang D. Transcriptome landscape of double negative T cells by single-cell RNA sequencing. J Autoimmun. 2021; 121:102653. Epub 2021 May 23. doi: 10.1016/j.jaut.2021.102653. PubMed PMID: 34022742.
Zheng H, Li X, Chen C, Chen J, Sun J, Sun S, Jin L, Li J, Sun S, Wu X, Quantum dot-based immunofluorescent imaging and quantitative detection of TOP2A and prognostic value in triple-negative breast cancer. International journal of nanomedicine. 2016; 11:5519-29. PMID: 27799773.
Zhou F. Molecular mechanisms of viral immune evasion proteins to inhibit MHC class I antigen processing and presentation. International reviews of immunology. 2009; 28 (5): 376-93.

### REFERENCES FOR EXAMPLE 18

1. Siegel, R. L., Miller, K. D., Fuchs, H. E. & Jemal, A. Cancer statistics. CA Cancer J. Clin 73, 17-48 (2023).
2. Prat, A. et al. Phenotypic and molecular characterization of the claudin-low intrinsic subtype of breast cancer. Breast Cancer Res. 12, 68-86 (2010).
3. Jemal, A. et al. Global cancer statistics. CA A Cancer J. Clin 61, 69-90 (2011).
4. Onitilo, A. A., Engel, J. M., Greenlee, R. T. & Mukesh, B. N. Breast Cancer Subtypes Based on ER/PR and Her2 Expression: Comparison of Clinicopathologic Features and Survival. Clin Med Res. 7, 4-13 (2009).
5.Harbeck, N. & Gnant, G. Breast cancer. Lancet 389, 1134-1150 (2017).
6. Gao, J. J. & Swan, S. M. Luminal A Breast Cancer and Molecular Assays: A Review. Oncologist 23, 556-565 (2018).
7. Lollini, P. L., Cavallo, F., Nanni, P. & Forni, G. Vaccines for tumour prevention. Nat. Rev. Cancer 6, 204-216 (2006).
8. Disis, M. L. et al. A multiantigen vaccine targeting neu, IGFBP-2, and IGF-IR pre-vents tumor progression in mice with preinvasive breast disease. Cancer Prev. Res. 6, 1273-1282 (2013).
9. Ebben, J. D., Lubet, R. A., Gad, E., Disis, M. L. & You, M. Epidermal growth factor receptor derived peptide vaccination to prevent lung adenocarcinoma formation: An in vivo study in a murine model of EGFR mutant lung cancer. Mol. Carcinog. 55, 1517-1525 (2016).
10. Pan, J. et al. Immunoprevention of KRAS-driven lung adenocarcinoma by a multipeptide vaccine. Oncotarget 8, 82689-82699 (2017).
11. Schneble, E. J. et al. The HER2 peptide nelipepimut-S(E75) vaccine (NeuVax™) in breast cancer patients at risk for recurrence: correlation of immunologic data with clinical response. Immunotherapy 6, 519-531 (2014).
12. Cecil, D. L. et al. Elimination of IL-10-inducing T-helper epitopes from an IGFBP-2 vaccine ensures potent antitumor activity. Cancer Res. 74, 2710-2718 (2014).
13. Disis, M. L. et al. Peptide-based, but not whole protein, vaccines elicit immunity to HER-2/neu, oncogenic self-protein. J. Immunol. 156, 3151-3158 (1996).
14. Klintman, M. et al. Changes in Expression of Genes Representing Key Biologic Processes After Neoadjuvant Chemotherapy in Breast Cancer, and Prognostic Implications in Residual Disease. Clin. Cancer Res. 15, 2405-2416 (2016).
15. Herschkowitz, J. I. et al. Identification of conserved gene expression features between murine mammary carcinoma models and human breast tumors. Gen-ome Biol. 8, R76 (2007).
16. Herschkowitz, J. & Lubet, R. Mouse models of triple negative [basal-like/claudin low] breast cancer. Breast Dis. 32, 63-71 (2010).
17. Aprelikova, O. et al. Development and Preclinical Application of an Immuno-competent Transplant Model of Basal Breast Cancer with Lung, Liver and Brain Metastases. PloS One 12, e0155262 (2016).
18. Butler, A., Hoffman, P., Smibert, P., Papalexi, E. & Satija, R. Integrating single-cell transcriptomic data across different conditions, technologies, and species. Nat. Biotechnol. 36, 411-420 (2018).
19. Stuart, T. et al. Comprehensive Integration of Single-Cell Data. Cell 177, 1888-1902 (2019).
20. Bachelier, R. et al. Effect of bilateral oophorectomy on mammary tumor formation in BRCA1 mutant mice. Oncol. Rep. 14, 1117-11120 (2005).
21. Carmona, S. J., Siddiqui, I., Bilous, M., Held, W. & Gfeller, D. Deciphering the transcriptomic landscape of tumor-infiltrating CD8 lymphocytes in B16 mela-noma tumors with single-cell RNA-Seq. Oncoimmunology 9, 1737369 (2020).
22. Oh, D. et al. Intratumoral CD4+ T Cells Mediate Anti-tumor Cytotoxicity in Human Bladder. Cancer. Cell 181, 1612-1625 (2020).
23. Yang, L. et al. Transcriptome landscape of double negative T cells by single-cell RNA sequencing. J. Autoimmun. 121, 102653 (2021).
24. Villar, J. & Segura, E. Decoding the Heterogeneity of Human Dendritic Cell Sub-sets. Trends Immunol. 41, 1062-1071 (2020).
25. Murphy, T. L. & Murphy, K. M. Dendritic cells in cancer immunology. Cell. Mol. Immunol. 19, 3-13 (2022).
26. Giladi, A. et al. Single-cell characterization of haematopoietic progenitors and their trajectories in homeostasis and perturbed haematopoiesis. Nat. Cell Biol. 20, 836-846 (2018).
27. Springer, I. et al. Prediction of Specific TCR-Peptide Binding From Large Dictionaries of TCR-Peptide Pairs. Front. Immunol. 11, 1803 (2020).
28. Pei, Y. F., Yin, X. M. & Liu, X. Q. TOP2A induces malignant character of pancreatic cancer through activating β-catenin signaling pathway. Biochim. Biophys. Acta Mol. Basis Dis. 1864, 197-207 (2018).
29. Zheng, H. et al. Quantum dot-based immunofluorescent imaging and quantitative detection of TOP2A and prognostic value in triple-negative breast cancer. Int. J. Nanomed. 11, 5519-5529 (2016).
30. Nakagawa, M. Expression of p53, Ki-67, E-cadherin, N-cadherin and TOP2A in triple-negative breast cancer. Anticancer Res. 31, 2389-2393 (2011).
31. Carey, L. A. et al. Race, breast cancer subtypes, and survival in the Carolina Breast Cancer Study. JAMA. 295, 2492-2502 (2006).
32. Siddharth, S. & Sharma, D. Racial Disparity and Triple-Negative Breast Cancer in African American Women: A Multifaceted Affair between Obesity, Biology, and Socioeconomic Determinants. Cancers 10, 514 (2018).
33. Green, J. E. et al. The C3 (1)/SV40 T-antigen transgenic mouse model of mammary cancer: ductal epithelial cell targeting with multistage progression to carcinoma. Oncogene 19, 1020-1027 (2000).
34. Park, K. H. et al. Insulin-like growth factor-binding protein-2 is a target for the immunomodulation of breast cancer. Cancer Res. 68, 8400-8409 (2008).
35. Mittendorf, E. A. et al. Vaccination with a HER2/neu peptide induces intra- and inter-antigenic epitope spreading in patients with early stage breast cancer. Surgery 139, 407-418 (2006)
36. Zhou, F. Molecular mechanisms of viral immune evasion proteins to inhibit MHC class I antigen processing and presentation. Int. Rev. Immunol. 28, 76-393 (2009).
37. Matsuo, M. et al. IFN-gamma enables cross-presentation of exogenous protein antigen in human Langerhans cells by potentiating maturation. Proc. Natl Acad. Sci. USA 101, 14467-14472 (2004).
38. Martinez-Lostao, L., Anel, A. & Pardo, J. How do cytotoxic lymphocytes kill cancer cells? Clin. Cancer Res. 21, 5047-5056 (2015).
39. Toussirot, E. The IL23/Th17 pathway as a therapeutic target in chronic inflammatory diseases. Inflamm Allergy Drug Targets 11, 159-168 (2012).
40. Lo, C. H. et al. Antitumor and Antimetastatic Activity of IL-23. J. Immunol. 171, 600-607 (2003).
41. Ma, X. et al. Interleukin-23 Engineering Improves CAR T Cell Function in Solid Tumors. Nat. Biotechnol. 38, 448-459 (2020).
42. Holzer, R. et al. Development and characterization of a progressive series of mammary adenocarcinoma cell lines derived from the C3 (1)/SV40 Large T-antigen transgenic mouse model. Breast Cancer Res. Treat 77, 65-76 (2003).

### NUMBERED CLAUSES

The disclosure can also be described in the following numbered clauses.

Clause 1. A composition, comprising one or more of: a polypeptide that is at least 80%, 85%, 90%, 95%, or 99% identical to SEQ ID NO: 1; a polypeptide that is at least 80%, 85%, 90%, 95%, or 99% identical to SEQ ID NO: 2; or a polypeptide that is at least 80%, 85%, 90%, 95%, or 99% identical to SEQ ID NO: 3.

Clause 2. The composition of clause 1, wherein the composition comprises the polypeptide that is at least 80%, 85%, 90%, 95%, or 99% identical to SEQ ID NO: 1.

Clause 3. The composition of clause 1 or 2, wherein the composition comprises the polypeptide that is at least 80%, 85%, 90%, 95%, or 99% identical to SEQ ID NO: 2.

Clause 4. The composition of any of clauses 1-3, wherein the composition comprises the polypeptide that is at least 80%, 85%, 90%, 95%, or 99% identical to SEQ ID NO: 3.

Clause 5. The composition of clause 1, wherein the composition comprises or consists of the polypeptide that is at least 80%, 85%, 90%, 95%, or 99% identical to SEQ ID NO: 1, the polypeptide that is at least 80%, 85%, 90%, 95%, or 99% identical to SEQ ID NO: 2, and the polypeptide that is at least 80%, 85%, 90%, 95%, or 99% identical to SEQ ID NO: 3.

Clause 6. The composition of clause 1, wherein the composition comprises or consists of the polypeptide having the sequence of SEQ ID NO: 1, the polypeptide having the sequence of SEQ ID NO: 2, and the polypeptide having the sequence of SEQ ID NO: 3.

Clause 7. The composition of clause 5, wherein the polypeptide that is at least 80%, 85%, 90%, 95%, or 99% identical to SEQ ID NO: 1, the polypeptide that is at least 80%, 85%, 90%, 95%, or 99% identical to SEQ ID NO: 2, and the polypeptide that is at least 80%, 85%, 90%, 95%, or 99% identical to SEQ ID NO: 3 are part of one continuous polypeptide.

Clause 8. The composition of clause 7, wherein a first linker is connected to the polypeptide that is at least 80%, 85%, 90%, 95%, or 99% identical to SEQ ID NO: 1 and the polypeptide that is at least 80%, 85%, 90%, 95%, or 99% identical to SEQ ID NO: 2, and/or wherein a second linker is connected to the polypeptide that is at least 80%, 85%, 90%, 95%, or 99% identical to SEQ ID NO: 2 and the polypeptide that is at least 80%, 85%, 90%, 95%, or 99% identical to SEQ ID NO: 3.

Clause 9. The composition of clause 6, wherein the polypeptide having the sequence of SEQ ID NO: 1, the polypeptide having the sequence of SEQ ID NO: 2, and the polypeptide having the sequence of SEQ ID NO: 3 are part of one continuous polypeptide.

Clause 10. The composition of clause 9, wherein a first linker is connected to the polypeptide having the sequence of SEQ ID NO: 1 and the polypeptide having the sequence of SEQ ID NO: 2, and/or wherein a second linker is connected to the polypeptide having the sequence of SEQ ID NO: 2 and the polypeptide having the sequence of SEQ ID NO: 3.

Clause 11. The composition of any one of clauses 1-10, further comprising a polypeptide having at least 80%, 85%, 90%, 95%, or 99% identity to one or more of SEQ ID NOs: 20-27.

Clause 12. The composition of any one of clauses 1-11, further comprising a polypeptide having at least 80%, 85%, 90%, 95%, or 99% identity to one or more of SEQ ID NOs: 28-30.

Clause 13. The composition of any of clauses 1-12, further comprising an adjuvant.

Clause 14. The composition of clause 13, wherein the adjuvant comprises a CpG oligo deoxynucleotide.

Clause 15. A composition comprising a polynucleotide encoding for one or more of: a polypeptide that is at least 80%, 85%, 90%, 95%, or 99% identical to SEQ ID NO: 1; a polypeptide that is at least 80%, 85%, 90%, 95%, or 99% identical to SEQ ID NO: 2; or a polypeptide that is at least 80%, 85%, 90%, 95%, or 99% identical to SEQ ID NO: 3.

Clause 16. The composition of clause 15, wherein the polynucleotide encodes for the polypeptide that is at least 80%, 85%, 90%, 95%, or 99% identical to SEQ ID NO: 1.

Clause 17. The composition of clause 15 or 16, wherein the polynucleotide encodes the polypeptide that is at least 80%, 85%, 90%, 95%, or 99% identical to SEQ ID NO: 2.

Clause 18. The composition of any of clauses 15-17, wherein the polynucleotide encodes the polypeptide that is at least 80%, 85%, 90%, 95%, or 99% identical to SEQ ID NO: 3.

Clause 19. The composition of clause 15, wherein the polynucleotide encodes the polypeptide that is at least 80%, 85%, 90%, 95%, or 99% identical to SEQ ID NO: 1, the polypeptide that is at least 80%, 85%, 90%, 95%, or 99% identical to SEQ ID NO: 2, and the polypeptide that is at least 80%, 85%, 90%, 95%, or 99% identical to SEQ ID NO: 3.

Clause 20. The composition of clause 15, wherein the polynucleotide encodes the polypeptide having the sequence of SEQ ID NO: 1, the polypeptide having the sequence of SEQ ID NO: 2, and the polypeptide having the sequence of SEQ ID NO: 3.

Clause 21. The composition of clause 19, wherein the polypeptide that is at least 80%, 85%, 90%, 95%, or 99% identical to SEQ ID NO: 1, the polypeptide that is at least 80%, 85%, 90%, 95%, or 99% identical to SEQ ID NO: 2, and the polypeptide that is at least 80%, 85%, 90%, 95%, or 99% identical to SEQ ID NO: 3 are part of one continuous polypeptide.

Clause 22. The composition of clause 21, wherein the polynucleotide encodes for a first linker that is connected to the polypeptide that is at least 80%, 85%, 90%, 95%, or 99% identical to SEQ ID NO: 1 and the polypeptide that is at least 80%, 85%, 90%, 95%, or 99% identical to SEQ ID NO: 2, and/or a second linker that is connected to the polypeptide that is at least 80%, 85%, 90%, 95%, or 99% identical to SEQ ID NO: 2 and the polypeptide that is at least 80%, 85%, 90%, 95%, or 99% identical to SEQ ID NO: 3.

Clause 23. The composition of clause 20, wherein the polypeptide having the sequence of SEQ ID NO: 1, the polypeptide having the sequence of SEQ ID NO: 2, and the polypeptide having the sequence of SEQ ID NO: 3 are part of one continuous polypeptide.

Clause 24. The composition of clause 23, wherein the polynucleotide encodes for a first linker that is connected to the polypeptide having the sequence of SEQ ID NO: 1 and the polypeptide having the sequence of SEQ ID NO: 2, and/or a second linker that is connected to the polypeptide having the sequence of SEQ ID NO: 2 and the polypeptide having the sequence of SEQ ID NO: 3.

Clause 25. The composition of any one of clauses 15-24, wherein the polynucleotide further encodes a polypeptide having at least 80%, 85%, 90%, 95%, or 99% identity to one or more of SEQ ID NOs: 20-27.

Clause 26. The composition of any one of clauses 15-25, wherein the polynucleotide further encodes a polypeptide having at least 80%, 85%, 90%, 95%, or 99% identity to one or more of SEQ ID NOs: 28-30.

Clause 27. The composition of any one of clauses 15-26, wherein the polynucleotide is mRNA.

Clause 28. The composition of any one of clauses 15-27, wherein the polynucleotide is present in a lipid nanoparticle.

Clause 29. A method of treating a subject, or a method for the preventative treatment of the subject, the method comprising: administering to the subject the composition of any of clauses 1-14 or the composition of any one of clauses 15-28.

Clause 30. The method of clause 29, wherein the subject has not previously been diagnosed with cancer or Triple Negative Breast Cancer (TNBC) and/or wherein the subject has not previously been treated for cancer or TNBC.

Clause 31. The method of clause 29, wherein the subject has been diagnosed with TNBC.

Clause 32. The method of clause 29, wherein the subject has been diagnosed with cancer and/or breast cancer.

Clause 33. The method of clause 32, wherein the breast cancer does not express one or more of: an estrogen receptor (ER), a progesterone receptor (PR), or human epidermal growth factor 2 (HER2).

Clause 34. The method of any of clauses 29-33, wherein the subject exhibits one or more risk factors associated with TNBC, wherein the one or more risk factors are selected from pregnancy, multiple child births, and obesity.

Clause 35. The method of any of clauses 29-34, wherein the administering comprises administering at least one dose of a therapeutically effective amount of the composition of any of clauses 1-14 or the composition of any one of clauses 15-28.

Clause 36. The method of clause 29, wherein the administering comprises administering at least two doses of a therapeutically effective amount of the composition of any of clauses 1-14 or the composition of any one of clauses 15-28.

Clause 37. The method of any of clauses 29-36, wherein the administering the composition at least partly results in an increased level of one or more of IFN-γ, TNF-α, IL-2, or IL-23 in the subject compared to an untreated control.

Clause 38. The method of clause 29, wherein the subject has not previously been diagnosed with cancer or lung cancer and/or wherein the subject has not previously been treated for cancer or lung cancer.

Clause 39. The method of clause 29, wherein the subject has been diagnosed with lung cancer.

Clause 40. The method of clause 29, wherein the subject has been diagnosed with cancer and/or lung cancer.

Clause 41. The composition of clause 1, wherein the polypeptide is at least 80%, 85%, 90%, 95%, or 99% identical to SEQ ID NO: 31.

Clause 42. The composition of clause 15, wherein the polynucleotide encodes for a polypeptide is at least 80%, 85%, 90%, 95%, or 99% identical to SEQ ID NO: 31.

In the foregoing description, it will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

All methods described herein can be performed in any suitable order unless otherwise indicated herein or otherwise clearly contradicted by context. The use of any and all examples provided herein, is intended merely to better illuminate the invention and does not pose a limitation on the scope of the invention unless otherwise claimed. No language in the specification should be construed as indicating any non-claimed element as essential to the practice of the invention.

Citations to a number of patent and non-patent references are made herein. The cited references are incorporated by reference herein in their entireties. In the event that there is an inconsistency between a definition of a term in the specification as compared to a definition of the term in a cited reference, the term should be interpreted based on the definition in the specification.

We claim:

1. A composition, comprising one or more of: a polypeptide that is at least 80%, 85%, 90%, 95%, or 99% identical to SEQ ID NO: 1; a polypeptide that is at least 80%, 85%, 90%, 95%, or 99% identical to SEQ ID NO: 2; or a polypeptide that is at least 80%, 85%, 90%, 95%, or 99% identical to SEQ ID NO: 3.

2.-6. (canceled)

7. The composition of claim 1, wherein the polypeptide that is at least 80%, 85%, 90%, 95%, or 99% identical to SEQ ID NO: 1, the polypeptide that is at least 80%, 85%, 90%, 95%, or 99% identical to SEQ ID NO: 2, and the polypeptide that is at least 80%, 85%, 90%, 95%, or 99% identical to SEQ ID NO: 3 are part of one continuous polypeptide.

8. The composition of claim 7, wherein a first linker is connected to the polypeptide that is at least 80%, 85%, 90%, 95%, or 99% identical to SEQ ID NO: 1 and the polypeptide that is at least 80%, 85%, 90%, 95%, or 99% identical to SEQ ID NO: 2, and/or wherein a second linker is connected to the polypeptide that is at least 80%, 85%, 90%, 95%, or 99% identical to SEQ ID NO: 2 and the polypeptide that is at least 80%, 85%, 90%, 95%, or 99% identical to SEQ ID NO: 3.

9.-10. (canceled)

11. The composition of claim 1, further comprising a polypeptide having at least 80%, 85%, 90%, 95%, or 99% identity to one or more of SEQ ID NOs: 20-27.

12. The composition of claim 1, further comprising a polypeptide having at least 80%, 85%, 90%, 95%, or 99% identity to one or more of SEQ ID NOs: 28-30.

13. The composition of claim 1, further comprising an adjuvant.

14. The composition of claim 13, wherein the adjuvant comprises a CpG oligo deoxynucleotide.

15. A composition comprising a polynucleotide encoding for one or more of: a polypeptide that is at least 80%, 85%, 90%, 95%, or 99% identical to SEQ ID NO: 1; a polypeptide that is at least 80%, 85%, 90%, 95%, or 99% identical to SEQ ID NO: 2; or a polypeptide that is at least 80%, 85%, 90%, 95%, or 99% identical to SEQ ID NO: 3.

16.-24. (canceled)

25. The composition of claim 15, wherein the polynucleotide further encodes a polypeptide having at least 80%, 85%, 90%, 95%, or 99% identity to one or more of SEQ ID NOs: 20-27.

26. The composition of claim 15, wherein the polynucleotide further encodes a polypeptide having at least 80%, 85%, 90%, 95%, or 99% identity to one or more of SEQ ID NOs: 28-30.

27. The composition of claim 15, wherein the polynucleotide is mRNA.

28. The composition of claim 15, wherein the polynucleotide is present in a lipid nanoparticle.

29. A method of treating cancer in a subject in need thereof, or a method for the preventative treatment of cancer in a subject in need thereof, the method comprising: administering to the subject the composition of claim 1.

30. (canceled)

31. The method of claim 29, wherein the subject in need thereof has been diagnosed with Triple Negative Breast Cancer (TNBC).

32.-33. (canceled)

34. The method of claim 29, wherein the subject in need thereof exhibits one or more risk factors associated with TNBC, wherein the one or more risk factors are selected from pregnancy, multiple child births, and obesity.

35.-36. (canceled)

37. The method of claim 29, wherein the administering the composition at least partly results in an increased level of one or more of IFN-γ, TNF-α, IL-2, or IL-23 in the subject compared to an untreated control.

38. (canceled)

39. The method of claim 29, wherein the subject in need thereof has been diagnosed with lung cancer.

40. (canceled)

41. The composition of claim 1, wherein the polypeptide is at least 80%, 85%, 90%, 95%, or 99% identical to SEQ ID NO: 31.

42. The composition of claim 15, wherein the polynucleotide encodes for a polypeptide is at least 80%, 85%, 90%, 95%, or 99% identical to SEQ ID NO: 31.

43. A method of treating cancer in a subject in need thereof, or a method for the preventative treatment of cancer in a subject in need thereof, the method comprising: administering to the subject the composition of claim 15.
